(19) United States
(12) Patent Application Publication
(10) Pub. No.: US 2026/0014538 A1
(43) Pub. Date: Jan. 15, 2026

(54) Granular Aerogel Scaffolds and Methods of Making and Using the Same

(71) Applicant: The Penn State Research Foundation, University Park, PA (US)

(72) Inventors: Amir Sheikhi, University Park, PA (US); Saman Zavari, University Park, PA (US); Sina Kheirabadi, University Park, PA (US)

(73) Assignee: The Penn State Research Foundation, University Park, PA (US)

(21) Appl. No.: 19/331,406

(22) Filed: Sep. 17, 2025

Related U.S. Application Data
(63) Continuation-in-part of application No. 19/208,226, filed on May 14, 2025.
Application No. 19/208,226 is a continuation-in-part of application No. 18/848,678, filed on Sep. 19, 2024, now Pat. No. 12,370,138.
(60) Provisional application No. 63/324,774, filed on Mar. 29, 2022.
(60) Provisional application No. 63/367,521, filed on Jul. 1, 2022.
(60) Provisional application No. 63/424,286, filed on Nov. 10, 2022.
(60) Provisional application No. 63/647,774, filed on May 15, 2024.

(51) Int. Cl. B01J 13/00; C08L 89/06; C12N 5/00

(52) U.S. Cl. CPC B01J 13/0091; C08L 89/06; C12N 5/0068; C08L 2205/22; C12N 2533/54; C12N 2537/10

(57) ABSTRACT

Embodiments relate to granular aerogel scaffolds and methods of making and using thereof. A method of forming granular aerogel scaffolds includes converting polymers or lipids to form hydrogel microparticles via a first crosslinking, assembling the hydrogel microparticles to form a granular hydrogel scaffold via a second crosslinking, and subjecting the granular hydrogel scaffold to supercritical carbon dioxide drying to form a granular aerogel scaffold.

### CROSS-REFERENCE TO RELATED APPLICATIONS

The present patent application is a continuation-in-part application of U.S. application Ser. No. 19/208,226, filed on May 14, 2025, which is a continuation-in-part application of U.S. application Ser. No. 18/848,678, now issued as U.S. Pat. No. 12,370,138, filed on Sep. 19, 2024, which is the U.S. National Stage Application of International Patent Application No. PCT/US2023/016658, filed on Mar. 29, 2023, which is related to and claims the benefit of priority of U.S. Provisional Application No. 63/324,774, filed on Mar. 29, 2022, and is further related to and clams the benefit of priority of U.S. Provisional Application No. 63/367,521, filed on Jul. 1, 2022, and is further related to and claims the benefit of priority of U.S. Provisional Application No. 63/424,286, filed on Nov. 10, 2022, the entire contents of which are incorporated by reference. U.S. application Ser. No. 19/208,226 is further related to and claims the benefit of priority of U.S. Provisional Application No. 63/647,744, filed on May 15, 2024, and is further related to and claims the benefit of priority of U.S. Provisional Application No. 63/647,774, filed on May 15, 2024. The present patent application is further related to and claims the benefit of priority of U.S. Provisional Application No. 63/695,930, filed on Sep. 18, 2024, the entire contents of which are incorporated by reference.

### STATEMENT REGARDING FEDERALLY SPONSORED RESEARCH DEVELOPMENT

This invention was made with government support under Grant Nos. NS121150 and HL167939 awarded by the National Institutes of Health. The Government has certain rights in the invention.

### FIELD OF THE INVENTION

Embodiments relate to hydrogel microparticles, bioactive granular hydrogel scaffolds, granular aerogel scaffolds, and methods of making and using thereof.

### BACKGROUND OF THE INVENTION

Biomaterials designed for tissue engineering and regenerative medicine have increasingly focused on incorporating porous structures that can effectively guide cell behavior and facilitate tissue regeneration. The pore characteristics of these materials, including size, interconnectivity, and porosity, are critical parameters that influence cell adhesion, infiltration, differentiation, and the exchange of nutrients and metabolic waste. Porous scaffolds that mimic the native extracellular matrix can create a pro-regenerative environment, promoting in situ tissue repair and integration. Various approaches have been explored to engineer pore microarchitecture, with the goal of optimizing biological performance and functional tissue formation.

Granular hydrogel scaffolds (GHS) have enabled rapid cell infiltration and downregulated inflammatory responses during tissue regeneration. Various polymers have been used as the building blocks of GHS, including hyaluronic acid (HA), polyethylene glycol (PEG), and gelatin methacryloyl (GelMA), via different crosslinking and assembly approaches. GelMA is methacryloyl-modified gelatin that can undergo physical (e.g., thermal) and chemical (e.g., free radical polymerization) crosslinking, providing a biocompatible network decorated with the cell-adhesive RGD motifs. GelMA GHS have been used for tissue engineering and 3D bioprinting.

Additionally, aerogels are a class of highly porous, lightweight biomaterials characterized by their large surface area and biocompatibility, making them suitable for a range of biomedical applications such as wound healing, bone regeneration, nerve repair, and drug delivery. Despite their advantageous properties, traditional aerogels often lack the ability to precisely control pore size and interconnectivity at the microscale, which limits their effectiveness in supporting rapid cell infiltration and tissue integration. Existing methods to modify pore characteristics, such as adjusting synthesis parameters, employing different drying techniques, or incorporating sacrificial materials, have limitations, including reduced control over pore microarchitecture, increased complexity, or the need for additional processing steps.

### BRIEF SUMMARY OF THE INVENTION

Embodiments relate to exemplary formulations and methods of converting protein/peptide-based materials and/or any other synthetic or semi-natural material, including carbohydrates and their derivatives, to granular hydrogel scaffolds. In some embodiments, scaffold formation does not require light exposure. A polymer can first be converted to stable microgels (micro-scale hydrogel particles) via chemical crosslinking (e.g., step 1), followed by microgel-microgel assembly using orthogonal, non-light-mediated crosslinking (e.g., step 2). Exemplary step 1 may involve a range of chemical crosslinking methods, such as free-radical polymerization of vinyl groups and any other technique. Exemplary step 2 may be based on the crosslinking of other functional groups that were not used in step 1, such as amines, using enzymes, dynamic covalent bond formation, or any other method.

Embodiments further relate to polymeric granular hydrogel scaffolds that may be formed inside of tissues that do not have access to light. Embodiments may provide additional, new opportunities for noninvasive or minimally invasive tissue regeneration using granular hydrogel scaffolds without requiring open surgery.

Embodiments further relate to microgels that may be decorated and/or encapsulated with other biological factors or nano-structure materials to further promote the biological function of granular hydrogels.

It is one object of the present disclosure to provide methods and formulations of converting polymers, such as proteins and/or peptides, to hydrogel microparticles that can form granular hydrogel scaffolds after injection in tissues.

It is a further object of the present disclosure to provide methods and formulations of crosslinking hydrogel microparticles in a way that they remain stable at the physiological temperature and undergo assembly after enzymatic activation to form granular hydrogel scaffolds.

It is a further object of the present disclosure to provide methods of crosslinking hydrogel microparticles such that they remain stable at the physiological temperature and undergo assembly after mixing with another polymer, such as aldehyde-modified hyaluronic acid, to form granular hydrogel scaffolds.

It is a further object of the present disclosure to provide methods and formulations of decorating hydrogel microparticles with biologics (e.g., growth factors, drugs, cells), followed by the hydrogel microparticle assembly to form granular hydrogel scaffolds with enhanced bioactivity (e.g., bioactive granular hydrogel scaffolds).

It is a further object of the present disclosure to provide methods and formulations of encapsulating biologics (e.g., growth factors, drugs, cells) in hydrogel microparticles, followed by hydrogel microparticle assembly to form bioactive granular hydrogel scaffolds.

It is a further object of the present disclosure to provide methods and formulations to form in situ granular hydrogel scaffold composites/nanocomposites that mimic the physicochemical and/or biological characteristics of native tissues, such as brain, skin, muscle, etc.

Embodiments further relate to porous microgels, porous hydrogel scaffolds, and methods of making and using thereof. It is one object of the present disclosure to provide porous microgels such that voids are incorporated into the scaffold structures. As the microgels serve as the building blocks of scaffolds, porous microgels may impart (or increase) porosity to the scaffold itself and may enhance the void fraction of the scaffold compared with scaffolds formed from nonporous microgels.

Embodiments further relate to hybrid (e.g., cell-microgel) aggregates. In particular, cells may serve as assembly engines and migrate/adhere to the microgels such that a self-assembly process is initiated and aggregates are formed. In particular, microgels may be significantly larger than the cells such that porous aggregates are formed. Porous aggregates may enhance molecular diffusion and improve cell viability.

Embodiments further relate to a new class of porous biomaterials termed granular aerogel scaffolds (GAS). These scaffolds are assembled from size-tunable GelMA microparticles, which are jammed and photocrosslinked to form GHS. Subsequent supercritical drying preserves the microarchitecture, resulting in GAS with engineered pore geometries and interconnected micron-scale void networks. Importantly, the pore structure and mechanical properties of the rehydrated GAS are comparable to those of the initial GHS, enabling consistent biological performance. Such embodiments may allow for the precise tuning of pore size and interconnectivity, independent of polymer concentration or crosslinking density, thereby overcoming key limitations of conventional aerogel fabrication techniques.

In an exemplary embodiment, a method of forming a granular aerogel scaffold comprises converting polymers or lipids to form hydrogel microparticles via a first crosslinking; assembling the hydrogel microparticles to form a granular hydrogel scaffold via a second crosslinking; and subjecting the granular hydrogel scaffold to supercritical carbon dioxide drying to form a granular aerogel scaffold.

In some embodiments, the method comprises converting polymers to form hydrogel microparticles via a first crosslinking.

In some embodiments, the polymers are selected from the group consisting of proteins, peptides, and carbohydrates.

In some embodiments, the polymers are gelatin methacryloyl.

In some embodiments, subjecting the granular hydrogel scaffold to supercritical carbon dioxide drying comprises replacing an aqueous phase of the granular hydrogel scaffold with an alcohol to form an alcogel; and subjecting the alcogel to supercritical carbon dioxide drying to form the granular aerogel scaffold.

In some embodiments, the first crosslinking comprises physical crosslinking.

In some embodiments, the first crosslinking comprises chemical crosslinking.

In some embodiments, the second crosslinking comprises physical crosslinking.

In some embodiments, the second crosslinking comprises chemical crosslinking.

In some embodiments, the method further comprises mixing the hydrogel microparticles with additional polymers and/or colloidal particles prior to assembling the hydrogel microparticles.

In some embodiments, the additional polymers are selected from the group consisting of aldehyde-modified carbohydrates, proteoglycans, and mixtures thereof.

In some embodiments, the method further comprises decorating the hydrogel microparticles with one or more selected from the group consisting of biologics, colloidal particles, and hybrid biologics-colloids prior to assembling the hydrogel microparticles.

In some embodiments, decorating the hydrogel microparticles comprises coating hydrogel microparticles with one or more selected from the group consisting of biologics, colloidal particles, and hybrid biologics-colloids.

In some embodiments, the biologics and/or colloidal particles and/or hybrid biologics-colloids are loaded to, attached on the surface of, or hybridized with nanocarriers bearing crosslinkable functional groups.

In some embodiments, the method further comprises encapsulating one or more selected from the group consisting of biologics, colloidal particles, and hybrid biologics-colloids in the hydrogel microparticles.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 shows an exemplary method for forming an embodiment of the granular hydrogel scaffolds.

FIG. 2A shows a schematic of individual gelatin methacryloyl hydrogel microparticle photocrosslinking and the mechanism of hydrogel microparticle assembly via activated factor XIII-mediated glutamyl-lysine bond formation.

FIG. 2B shows gelatin methacryloyl droplets and hydrogel microparticles (photocrosslinked droplets), prepared in three sizes: small, medium, or large (scale bar is 200 μm).

FIG. 2C shows the average diameter of gelatin methacryloyl droplets and hydrogel microparticles.

FIG. 2D shows the orthographic view and pore identification of gelatin methacryloyl granular hydrogel scaffolds, fabricated using small, medium, or large hydrogel microparticles (scale bar is 200 μm).

FIG. 2E shows gelatin methacryloyl granular hydrogel scaffolds void fraction.

FIG. 2F shows equivalent median pore diameter of gelatin methacryloyl granular hydrogel scaffolds.

FIG. 2G shows injectability of packed gelatin methacryloyl hydrogel microparticles (scale bar is 200 μm).

FIG. 3A shows optical images of small, medium, or large photocrosslinked gelatin methacryloyl hydrogel microparticles with gelatin methacryloyl concentration of 1.5% (w/v) and 60 s of UV exposure at the intensity of 15 mW cm⁻² incubated at 37° C. for up to 24 h (scale bar is 200 μm).

FIG. 3B shows diameters of hydrogel microparticles.

FIG. 4A shows frequency sweep tests to measure the storage modulus of bulk gelatin methacryloyl hydrogel (concentration=1, 1.5, 2, or 3% w/v in 4-(2-hydroxyethyl)-1-piperazineethanesulfonic acid, resembling hydrogel microparticles), photocrosslinked via UV light exposure for 30 s.

FIG. 4B shows frequency sweep tests to measure the storage modulus of bulk gelatin methacryloyl hydrogel (concentration=1, 1.5, 2, or 3% w/v in 4-(2-hydroxyethyl)-1-piperazineethanesulfonic acid, resembling hydrogel microparticles), photocrosslinked via UV light exposure for 60 s.

FIG. 4C shows the storage modulus of bulk gelatin methacryloyl scaffolds at a frequency of 1 rad s- and oscillatory strain of 0.1%. The data represents mean±standard deviation for at least 3 samples.

FIG. 4D shows compressive modulus of gelatin methacryloyl granular hydrogel scaffolds assembled via reacting medium gelatin methacryloyl hydrogel microparticles with activated factor XIII (concentrations of 0, 2.5, 5, or 10 U mL⁻¹) for 1.5 h. The data represents mean±standard deviation for at least 3 samples.

FIG. 4E shows the effect of activated factor XIII (5 U mL⁻¹) reaction time at 37° C. on the compressive modulus of gelatin methacryloyl granular hydrogel scaffolds. The data represents mean±standard deviation for at least 3 samples.

FIGS. 5A-C show in situ fabrication of granular hydrogel scaffolds from thermoresistive GelMA microgels (scale bar is 200 μm).

FIGS. 6A-D show material and mechanical characterization of granular hydrogel scaffolds made from thermoresistive GelMA microgels.

FIGS. 7A-C show biocompatibility assessment of granular hydrogel scaffolds made from thermoresistive GelMA microgels.

FIG. 8 shows an exemplary method for regenerating tissue via the granular hydrogel scaffold.

FIG. 9 is a schematic illustration showing droplets of a homogenous binary solution of GelMA (7% w/v) and PEG (1.5, 2, or 2.5% w/v) polymers at Tᵢ, fabricated using a high-throughput step emulsification microfluidic device. The scale bar is 200 μm.

FIG. 10 is a schematic illustration showing droplets converted to microgels by reducing temperature to T_{f}, initiating the thermally induced phase separation of GelMA and PEG. The phase separation is driven by the miscibility reduction of GelMA and PEG in an aqueous solution, yielding two distinct phases until halted by GelMA polymer physical gel formation.

FIG. 11 is a schematic illustration showing phase separation temperature, Tₚs, of GelMA-PEG mixtures at varying PEG concentrations (1.5, 2, 2.5% w/v) and a fixed GelMA concentration (7% w/v).

FIG. 12 is a schematic illustration demonstrating that the removal of oil and surfactant enables PEG chains to diffuse out of the physically crosslinked GelMA microgels (shown with arrows), leading to the formation of interconnected pores and rendering the microgels porous.

FIG. 13 is a schematic illustration showing GHS with a hierarchical porous structure may be fabricated via the free radical photopolymerization of jammed porous microgels, forming intra- and inter-microgel covalent bonds.

FIG. 14 shows bright-field microscopy images of physically-crosslinked microgels with varying porosity, fabricated via the phase separation of GelMA (7% w/v) and PEG polymer mixtures at varying PEG concentrations (1.5, 2, or 2.5% w/v) and T_{f}. Microgel size (average diameter ˜187±10 μm, number of analyzed microgels n>5000) is independent from microgel porosity, and the particles have a narrow size distribution. The dashed and solid lines show the median and quartiles in the datasets, respectively. The scale bar is 200 μm.

FIG. 15 shows fluorescence microscopy images of individually photocrosslinked microgels that initially contained PEG (2% w/v), incubated with FITC-dextran (average molecular weight=2 MDa). Microgels are shown in cross-sectional 2D slices and 3D images that are constructed from Z-stacks. The scale bar is 50 μm.

FIG. 16 is a graph showing average median pore size of porous microgels (initially contained 2% w/v PEG), calculated using a MATLAB script (n>15).

FIG. 17 is a graph showing void fraction of porous microgels (initially contained 2% w/v PEG), calculated using a MATLAB script (n>15).

FIG. 18 shows fluorescence images of GHS, made up of microgels with varying pore sizes, acquired using a fluorescent molecule (FITC-dextran, average molecular weight=2 MDa). The scale bar is 100 μm.

FIG. 19 is a graph showing void fraction (n=5) of GHS, fabricated using the porous or nonporous microgels. The analysis was conducted using a MATLAB code.

FIG. 20 is a graph showing pore size (n=5) of GHS, fabricated using the porous or nonporous microgels. The analysis was conducted using a MATLAB code.

FIG. 21 is a graph showing compressive stress-strain curves of scaffolds.

FIG. 22 is a graph showing compressive modulus of GHS with hierarchical (GHS-S and GHS-L) and non-hierarchical (GHS-N) porous structures (n=5).

FIG. 23 is a graph showing dynamic moduli of GHS, fabricated using porous or nonporous microgels versus angular frequency.

FIG. 24 is a graph showing G′ acquired at angular frequency ˜1 rad s⁻¹ and strain ˜0.1% (n=5).

FIG. 25 is a graph showing G″, acquired at angular frequency ˜1 rad s⁻¹ and strain 0.1% (n=5).

FIG. 26 is a schematic illustration showing individual microgel photocrosslinking, followed by mixing with NIH/3T3 murine fibroblast cells and culturing.

FIG. 27 shows NIH/3T3 murine fibroblast cells, stained with calcein AM, imaged via fluorescence microscopy. The 2D slices and 3D-rendered images of microgels, showing cell adhesion to the non-porous or porous microgel exterior as well as cell infiltration into the porous microgels. The scale bar is 100 μm.

FIG. 28 is a graph showing cell volume, calculated by the summation of total cell area in each 2D image multiplied by the Z-step size in each microgels, assessed using a MATLAB code (number of analyzed microgels per group n>15). Dashed and solid lines indicate the median and quartiles, respectively.

FIG. 29 shows a live/dead assay, conducted at varying culture periods. The scale bar is 500 μm.

FIG. 30 shows graphs demonstrating cell viability in cell-laden GHS. A high cell viability (>95%) is observed within 7 days of culture for all the study groups (n=3) (top), and metabolic activity of NIH/3T3 murine fibroblast cells in the scaffolds, measured using the PrestoBlue assay (bottom). In all study groups, metabolic activity significantly increased on days 4 and 7 compared with day 1 (n=5).

FIG. 31 is a schematic illustration of the subcutaneous implantation mouse model to evaluate endogenous cell infiltration into acellular scaffolds. Dashed lines indicate varying scaffold depths.

FIG. 32 shows representative images of stained cell nuclei, showing the distribution of infiltrated cells (labeled with DAPI) into the scaffolds, acquired 2 weeks after scaffold implantation. Dashed lines show the tissue-scaffold interface. The scale bar is 200 μm.

FIG. 33 is a graph showing cell density in GHS, measured as the ratio of DAPI-stained nucleus area over the total ROI area. A higher cell density is observed in GHS-L and GHS-S compared with GHS-N.

FIG. 34 shows graphs demonstrating cell infiltration profile at varying depths of (left) GHS-N, (center) GHS-S, and (right) GHS-L, normalized with the total ROI area (length ˜400 μm and height ˜200 μm). The highest cell density across all study group is yielded in the top 200 μm layer adjacent to the tissue, and no significant changes are observed thereafter.

FIG. 35 shows images of infiltrated cells, stained with α-SMA, CD31, or CD68. Myofibroblasts, endothelial cells, and macrophages are confirmed across all study groups. White dashed lines indicate the tissue-scaffold interface. The scale bar is 200 μm.

FIG. 36 shows graphs demonstrating coverage area of infiltrated cells, stained with (top) α-SMA, (middle) CD31, or (bottom) CD68 in varying study groups. A significantly higher CD31 fluorescence area is observed in GHS-S and GHS-L compared with GHS-N. Additionally, α-SMA fluorescence area in GHS-L is significantly higher than that in GHS-N.

FIG. 37 is a schematic illustration showing GelMA droplets photochemically crosslinked to form stable microgels.

FIG. 38 shows GelMA droplets and their corresponding microgels formed via photocrosslinking in three distinct sizes: small (left), medium (center), and large (right). Fluorescent images show the size distribution of droplets and resulting microgels. Histograms presenting the relative frequency (%) of each droplet and microgel size.

FIG. 39 is a schematic illustration demonstrating that interactions between cells and ECM or GelMA influence the formation of cell aggregates, including cell spheroid and BHS. The adhesion is regulated by homophilic binding between cadherin moieties, and integrin-mediated cell-biomaterial and cell-ECM interactions.

FIG. 40 is a schematic illustration showing NIH/3T3 murine fibroblast cells and GelMA microgels are mixed to initiate cell-mediated microgel assembly (step 1). Initially, cells adhere to the microgels because of the adhesive moieties on GelMA (step 2). CS are formed by cell-cell aggregation, and BHS are yielded by cell-microgel assembly (step 3). Pseudo-colored optical microscopy images of cells and GelMA microgels, showing the formation of two types of aggregates.

FIG. 41 shows optical microscopy images NIH/3T3 murine fibroblast cells cultured with varying microgel sizes on a planar, non-adhesive substrate for 72 h. The scale bar is 300 μm.

FIG. 42 shows graphs demonstrating representative x-y trajectories and relative angles for the selected microgel pairs. The black solid lines denote the trajectories of the center of mass, and the line segments denote the relative angle θ between two microgels.

FIG. 43 is a graph showing remaining single cell percentage for small, medium, and large microgels. Inset: the same data plotted in logarithmic scale. The exponential fits are shown by the solid lines.

FIG. 44 is a graph showing characteristics decay time (τ) for the formation kinetics of BHS with varying sizes of microgels.

FIG. 45 is a graph showing the equivalent radii R_{eq} of BHS, formed using the small, medium, or large microgels after 72 h of culture.

FIG. 46 shows schematic illustration of BHS or cell spheroids formed by mixing cells with varying sizes of microgels in a U-bottom microwell plate (left). Confocal microscopy images of BHS or cell spheroid after 5 days, with actin filaments, nuclei, and microgels. (left-center). Nucleus density heatmap correlated with confocal microscopy images. (right-center) SEM images of aggregates, showing that cell spheroid and BHS-S have compact spherical shapes, while BHS-M and BHS-L were porous because of the larger building blocks. (right)

FIG. 47 is a graph showing metabolic activity of cells in aggregates, showing significantly higher values in BHS-M and BHS-L compared with cell spheroid and BHS-S. Two-way ANOVA is performed with Tukey's post-hoc multiple comparisons. NS=not significant with p≥0.05, **p<0.01, ****p<0.0001. For comparing days 7 with 1 of each sample, ns=not significant with p≥0.05, ##p<0.01, ####p<0.0001.

FIG. 48 is R_{eq} heatmap of different aggregates during the initial 24 h.

FIG. 49 is R_{eq} heatmap of aggregates from day 1 to 5.

FIG. 50 is a graph showing optical microscopy images of cell spheroids (top block) or BHS-M (bottom block), each comprising HUVEC, MSC, or HUVEC+MSC in the top, middle, and bottom rows, respectively, over five days.

FIG. 51 is a graph showing R_{eq} measured at day 1, comparing cell spheroids or BHS-M, for HUVEC, MSC, and HUVEC+MSC.

FIG. 52 is a graph showing R_{eq} measured at day 3, comparing cell spheroids or BHS-M, for HUVEC, MSC, and HUVEC+MSC.

FIG. 53 is a graph showing R_{eq} measured at day 5, comparing cell spheroids or BHS-M, for HUVEC, MSC, and HUVEC+MSC.

FIG. 54 shows schematic illustrations and fluorescence images of building blocks, including cells and microgels, cell spheroids, cell spheroids and microgels, or BHS, cultured for 72 h to form tissue-like constructs. Upon transferring and gentle pipetting, only the cell spheroids or BHS maintain their structural integrity and cohesion, while the other two groups disintegrate (left)), and further shows radar plots, comparing the advantages of BHS as building blocks for the in vitro fabrication of tissue-like structures based on cell-matrix interactions, scalability, structural integrity, cell viability and activity, modularity, and building block fusion (right).

FIG. 55 shows an exemplary method for forming an embodiment of the granular aerogel scaffolds.

FIG. 56 is a schematic illustration of an exemplary GAS fabrication process using GelMA droplets. Droplets are physically crosslinked to yield hydrogel microparticles, which are packed and chemically crosslinked to form GHS. GHS undergo solvent exchange to form granular alcogel scaffolds, followed by SCD to yield GAS.

FIG. 57 is a schematic illustration of GelMA droplet fabrication in a step-emulsification microfluidic device.

FIG. 58 shows Brightfield microscopy images of fabricated GelMA droplets with varying sizes. The scale bar is 100 μm.

FIG. 59 shows SEM images of the GAS, fabricated using varying sizes of microparticles. The scale bar is 50 μm.

FIG. 60 is a graph showing compressive modulus of S-, M-, and L-GAS.

FIG. 61 shows micro-CT imaging and analysis of M-GAS, showing (i) a 3D rendered scaffold image, (ii) detected pores, and (iii) pore interconnectivity. Spheres and bars indicate individual pores and pore throats, respectively. The scaffold diameter ˜620 μm and height ˜380 μm. One-way ANOVA is performed, followed by the Tukey's post-hoc multiple comparison test. **p<0.01 and ****p<0.0001.

FIG. 62 shows images of S-, M-, and L-GAS swelling in DPBS at 37° C. over time. The scale bar is 5 mm.

FIG. 63 is a graph showing the swelling ratio of (left) S-GAS, (middle) M-GAS, and (right) L-GAS at different time points. One-way ANOVA is performed, followed by the Tukey's post-hoc multiple comparison test. **p<0.01, ***p<0.001, and ****p<0.0001.

FIG. 64 shows fluorescence images of rGAS, acquired to visualize scaffold void spaces using FITC-dextran (Mw ˜2 MDa). Raw 2D fluorescence images, presenting one layer of scaffolds at which microparticles have maximum contact, which are used to measure the median equivalent pore diameter using a MATLAB code. Fluorescence 3D images with a Z-depth of ˜260, ˜168, or ˜65 μm for rL-, rM-, or rS-GAS, respectively, used to calculate VVF via dividing the void space volume by the total volume of imaged section. The scale bar is 100 μm.

FIG. 65 is a graph showing VVF of rS-, rM-, or rL-GAS.

FIG. 66 is a graph showing medium equivalent pore diameter of rS-, rM-, or rL-GAS.

FIG. 67 includes graphs showing (left) storage modulus of GelMA rGAS, fabricated using varying microparticle sizes, versus angular frequency at shear strain of 0.1%, and (right) average storage modulus of rGAS at angular frequency of 1 rad s⁻¹ and shear strain of 0.1%.

FIG. 68 includes graphs showing (left) compressive stress versus compressive strain, measured via the compression test, and (right) compressive modulus of rGAS, calculated within the linear region of the compressive stress-strain curve.

FIG. 69 shows live/dead fluorescence images of topically seeded NIH/3T3 murine fibroblast cells on M-GHS and M-GAS on days 1, 4, and 7 after seeding. The scale bar is 100 μm.

FIG. 70 is a graph showing cell viability in M-GHS and M-GAS, demonstrating no toxicity.

FIG. 71 is a graph showing cell metabolic activity on days 1, 4, and 7 in M-GHS and M-GAS. Metabolic activity in both study groups increases.

FIG. 72 shows counter gravity capillary-driven cell drawing (day 0) and cell migration (day 3) in M-GHS, rM-GAS, and M-GAS. Dashed lines show the bottom of scaffolds and the average cell drawing/migration length into the scaffolds. There is no cell drawing/migration from the bottom surface of M-GHS and rM-GAS after 3 days. The scale bar is 200 μm.

FIG. 73 is a graph showing average cell drawing/migration length from the bottom into M-GAS.

FIG. 74 is a schematic illustration of A schematic of subcutaneously implanted GAS in two incised pockets on the mouse dorsal skin, undergoing endogenous cells infiltration. Dashed lines indicate 200 μm intervals, measured from the tissue-scaffold interface.

FIG. 75 shows the DAPI-stained nucleus of cells infiltrated into S-, M-, or L-GAS, imaged via confocal microscopy. The scale bar is 100 μm.

FIG. 76 is a graph showing density of infiltrated cells in S-, M-, or L-GAS, measured as the ratio of infiltrated cell nucleus area over the toral ROI area.

FIG. 77 includes graphs showing cell density in 200 μm intervals, starting from the scaffold-tissue interface, normalized with the total cell density in each ROI for (left) S-, (middle) M-, and (right) L-GAS.

FIG. 78 is a graph showing cell nucleus area at varying layers of (left) L-GAS and (right) M-GAS, normalized with the average nucleus area in the corresponding layer of S-GAS.

FIG. 79 is a graph showing cell nucleus area at varying depths of L-GAS, normalized with the average nucleus area in the same depth of M-GAS.

FIG. 80 shows Immunofluorescence staining of varying cell populations, including α-SMA⁺ (myofibroblasts), CD31⁺ (endothelial cells), CD68⁺ (macrophages), CD86⁺ (M1 macrophages), CD206⁺ (M2 macrophages), and CD11b⁺ (leukocytes), infiltrated in the GelMA GAS comprising varying microparticle sizes. Dashed lines present the scaffold-tissue interface. The scale bar is 100 μm.

FIG. 81 includes graphs showing quantitative analyses of the occupied area by (top left) α-SMA⁺, (top right) CD31⁺, (middle left) CD68⁺, (middle right) CD86⁺, (bottom left) CD206⁺, or (bottom right) CD11b⁺ cells in each ROI.

FIG. 82 shows cross-sectional SEM images of GAS, fabricated using varying sizes of microparticles. The scale bars for the 100×, 250×, and 2000× magnifications are 500, 500, and 50 μm, respectively

FIG. 83 shows Brightfield images of S- and L-GHS incubated in DPBS (before ethanol gradient) and after a treatment with the ethanol gradient (30, 50, 60, 70, 80, 90, and 95% v/v in ultrapure water). In DPBS, irrespective of hydrogel microparticle size, the covalently assembled hydrogel microparticles are in contact with each other, and no observable neck is formed. After undergoing the ethanol gradient, microparticles deform, and intraparticle contact surfaces change, leading to neck formation, with greater deformation observed in granular alcogel scaffolds composed of smaller microparticles. Red arrows indicate intraparticle contact areas. The scale bars in the main and inset images are 50 and 20 μm, respectively.

FIG. 84 shows Brightfield images of granular alcogel scaffolds and SEM images of GAS, fabricated using varying GelMA concentrations (2 and 10% w/v). At a lower GelMA concentration, intraparticle neck formation is more pronounced and higher deformation is yielded. The scale bar is 100 μm.

FIG. 85 shows SEM images of dried GHS using various drying techniques, including SCD to produce M-GAS, ambient pressure drying at room temperature or 75° C. to produce GXS, and freezing at −80° C. and −196° C., followed by lyophilization to fabricate GCS. Red arrows show microparticle-microparticle contact surfaces in GXS. The scale bars are 1 mm, 500 μm, or 50 μm for the magnification of 100×, 250×, or 2000×, respectively.

FIG. 86 includes graphs showing swelling ratio of small S-, M-, and L-GAS (equivalent scaffold diameter ˜2.1-3.0 mm and height ˜1.5 mm). The smaller the microparticles, the faster the GAS swell.

FIG. 87 shows images of a GelMA bulk aerogel scaffold and its swollen state in DPBS at 37° C. and varying time points. The scale bar is 5 mm. The dashed circles indicate the periphery of dry and swelling scaffolds.

FIG. 88 is a graph showing swelling ratio of GelMA bulk aerogel scaffolds over time. Scaffolds reach the maximum swelling ratio after ˜1 h of incubation.

FIG. 89 shows fluorescence images of GHS, acquired to visualize scaffold void spaces using FITC-dextran (M_{w}˜2 MDa). Raw 2D fluorescence images present a layer of scaffolds at which hydrogel microparticles have the maximum contact. These images are used to measure the median equivalent pore diameter using a MATLAB code. Fluorescence 3D images with a Z-depth of ˜260, ˜168, or ˜65 μm for L-, M-, or S-GHS, respectively, used to calculate VVF via dividing the void space volume by the total volume of imaged section. The scale bar is 100 μm.

FIG. 90 includes graphs showing (left) VVF and (right) the median equivalent pore diameter of GHS and rGAS, fabricated using varying hydrogel microparticle sizes.

FIG. 91 includes graphs showing oscillatory strain sweep test results for (top left) rS-GAS, (top right) rM-GAS, (middle left) rL-GAS, (middle right) S-GHS, (bottom left) M-GHS, and (bottom right) L-GHS. These tests are performed at a constant frequency (1 rad s⁻¹) to identify the linear viscoelastic region (LVR) of hydrated scaffolds.

FIG. 92 includes graphs showing (left) storage modulus of GHS, fabricated using varying hydrogel microparticle sizes versus angular frequency, conducted at oscillatory shear strain ˜0.1%, and (right) average storage modulus of study groups at angular frequency of 1 rad s⁻¹ and shear strain of 0.1%.

FIG. 93 includes graphs showing (left) loss modulus of rGAS versus angular frequency at shear strain of 0.1%, and (right) average loss modulus of rGAS at angular frequency of 1 rad s⁻¹ and shear strain of 0.1%.

FIG. 94 includes graphs showing (left) loss modulus of GHS fabricated using varying hydrogel microparticle sizes versus angular frequency, and (right) average loss modulus of study groups at angular frequency of 1 rad s⁻¹ and shear strain of 0.1%.

FIG. 95 includes graphs showing (left) compressive stress versus compressive strain, measured via the compression test, and (right) compressive modulus of GHS calculated using the linear region of compressive stress-strain curve, i.e., within ˜0.05-0.15 mm mm⁻¹.

FIG. 96 is a graph showing cell metabolic activity fold change on days 4 and 7 with respect to the average metabolic activity on day 1 in M-GHS and M-GAS. Both study groups undergo an increase in metabolic activity fold change, and the increase in M-GAS is significantly higher than in M-GHS.

FIG. 97 shows fluorescence images from the sagittal plane of M-GHS and M-GAS, topically seeded with fluorescently labeled NIH/3T3 murine fibroblast cells on days 0 (˜4 hours after seeding) and 3. The scale bar is 200 μm.

FIG. 98 is a graph showing average cell drawing length, driven by capillary and gravitational forces on day 0, and the average cell migration length in the scaffolds after 3 days. Significant cell drawing/migration is observed in M-GAS compared with M-GHS on both days.

FIG. 99 shows fluorescence images from the sagittal plane of GelMA bulk hydrogel and aerogel scaffolds, topically seeded with fluorescently labeled NIH/3T3 murine fibroblast cells on days 0 (˜4 hours after seeding) and 3. The scale bar is 100 μm.

FIG. 100 shows counter gravity capillary-driven cell drawing (day 0, ˜4 h after cell seeding) and cell migration (day 3) in S-, M-, or L-GAS. Dashed lines show the bottom of scaffolds and average cell drawing/migration length into the scaffolds. The scale bar is 200 μm.

FIG. 101 is a graph showing average cell drawing/migration length from the bottom into S-, M-, or L-GAS.

FIG. 101 is a graph showing average cell drawing/migration length from the bottom into S-, M-, or L-GAS.

FIG. 102 includes images showing cellular infiltration and ECM deposition within the scaffolds one week after implantation. Arrows show cells infiltrating the inter-microgel void spaces, and the dashed line indicates the tissue-scaffold interface. The scale bar is 50 μm.

FIG. 103 includes graphs showing a comparison between the CD86⁺ and CD206⁺ cell coverage area in S-, M-, or L-GAS.

### DETAILED DESCRIPTION OF THE INVENTION

The following description is of embodiments presently contemplated for carrying out the present invention. This description is not to be taken in a limiting sense but is made merely for the purpose of describing the general principles and features of the present invention. The scope of the present invention should be determined with reference to the claims.

Embodiments relate to bioactive granular hydrogel scaffolds configured for use in tissue engineering and three-dimensional (3D) bioprinting. For example, scaffolds may mimic the mechanical and biological properties of tissues such that, once a scaffold is integrated with host tissue, the scaffold may provide a supportive environment to the tissue. More specifically, cells can migrate from surrounding host tissue into the scaffold. Once cells are adhered to or otherwise integrated with the scaffold, the cells may proliferate and enable tissue growth or regeneration.

Granular hydrogel scaffolds may be formed from jamming (e.g., packing) hydrogel microparticles, followed by crosslinking the hydrogel microparticles through covalent and/or noncovalent intermediate bond formation. The hydrogel microparticles may therefore serve as the building blocks of the scaffolds. The terms “hydrogel microparticles” and “microgels” may be used interchangeably herein. The terms “granular hydrogel scaffolds” and “scaffolds” may similarly be used interchangeably herein.

The microgels may be formed by crosslinking one or more polymers or lipids. Polymers may be selected from the group consisting of proteins, peptides, carbohydrates, or any other natural, semi-natural, or synthetic polymeric materials, and mixtures thereof. In some embodiments, polymers may be selected from the group consisting of hyaluronic acid (HA), polyethylene glycol (PEG), gelatin methacryloyl (GelMA), and mixtures thereof.

In some embodiments, the microgels may be formed from a polymer blend. As used herein, the term “polymer blend” may refer to a homogeneous mixture of two or more polymers, such as a physical mixture of two or more polymers held together by intermolecular forces, without chemical bonds linking therebetween. In some embodiments, a polymer blend may include two or more polymers selected from the group consisting of HA, PEG, and GelMA.

In some embodiments, the polymers may be crosslinked to form the microgels via physical crosslinking and/or chemical crosslinking. Examples of chemical crosslinking include free radical polymerization (e.g., free radical polymerization of vinyl groups) or any other suitable chemical crosslinking techniques.

In some embodiments, the microgels may be nonporous, such that the microgels may not include significant voids or pores in their structures.

In some embodiments, the microgels may be porous, such that voids are incorporated into the structures. As the microgels serve as the building blocks of scaffolds, porous microgels may impart (or increase) porosity to the scaffold itself and may enhance the void fraction of the scaffold compared with scaffolds formed from nonporous microgels.

In some embodiments, the microgel voids may be between 5 and 40 μm.

As used herein, the term “void fraction” refers to a measurement used to quantify the fraction of the total microgel volume that is occupied by voids (or empty spaces).

Voids formed within scaffolds may advantageously promote cell infiltration and host tissue integration. While the use of nonporous microgels may enable inter-microgel pores (e.g., voids between microgels), the void fraction of the resulting scaffold is limited to that of random packing of the microgels. Accordingly, porous microgels may not only attain inter-microgel pores, but also necessarily provide intra-microgel pores (e.g., voids within the microgels themselves), thereby increasing the void fraction of the resulting scaffold. Cell infiltration and host tissue integration may therefore be increased. Further, cell distribution within the scaffolds may similarly be enhanced, thus leading to a more unfirm scaffold in comparison to scaffolds formed from nonporous microgels.

In some embodiments, the microgels and/or scaffolds may have a void fraction between 15 and 60%. Void fractions above 60% may inhibit mechanical strength of the resulting scaffold, while void fractions below 15% may not enable improved cell infiltration and host tissue integration, among other benefits.

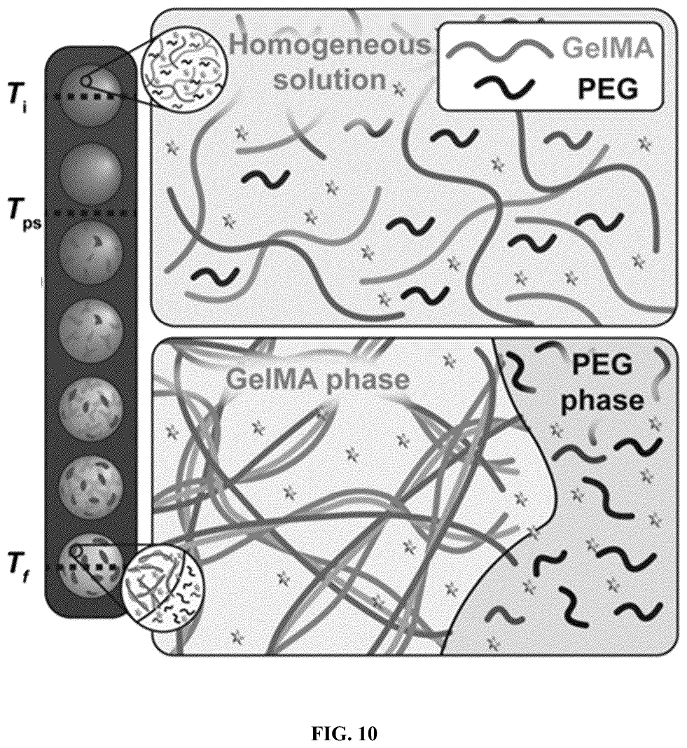

Porous microgels may be formed using any suitable technique. Referring to FIGS. 10-12, in one exemplary method of forming porous microgels, at least a first polymer and a second polymer may be crosslinked and provided as a composite microgel suspension in a liquid solution at an initial temperature. The composite microgel suspension may be thermally phase separated into two distinct phases by lowering the temperature of the suspension to a final temperature, in which the final temperature is lower than the phase separation temperature of the polymer mixture. As used herein, the term “phase separation temperature” refers to a temperature in which a first polymer and a second polymer exhibit a reduction in miscibility and yield two distinct phases, such that the second polymer may be fully or partially separated from the first polymer.

In particular, the thermodynamic incompatibility between the first polymer and the second polymer may lead to liquid-liquid phase separation. The microgel suspension may then be filtered from the liquid solution, after which the second polymer may diffuse out of the microgels, resulting in pores within the individual microgels composed of the first polymer. Porous microgels may therefore be provided via thermally induced polymer phase separation.

In some embodiments, the first polymer is GelMA and the second polymer is PEG, such that the resulting porous microgels comprise GelMA after PEG release.

The microgels may be any shape, size, and/or aspect ratio. In some embodiments, at least a portion of the microgels have a spherical shape. In some embodiments, at least a portion of the microgels have a rod-like shape. In some embodiments, the size of the microgels is between 10-200 μm. In a preferred embodiment, the microgels may have an aspect ratio between 1-10.

In some embodiments, the microgels may be crosslinked to form the scaffolds. For example, the scaffolds may be formed via free radical photopolymerization of jammed porous microgels after exposure to light, forming intra- and/or inter-microgel covalent bonds.

In alternative embodiments, the scaffolds may not require light exposure for scaffold formation and may be formed inside of tissues that do not have access to light. These embodiments may be advantageous as they may allow for noninvasive or minimally invasive tissue regeneration, vascularization inducement, axonogensis inducement, and/or tissue function improvement techniques using the scaffolds without requiring open surgery.

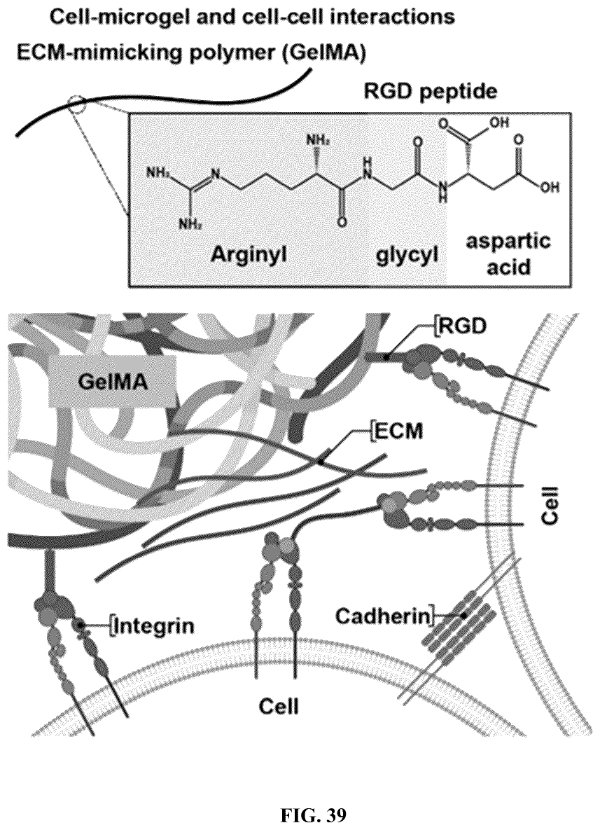

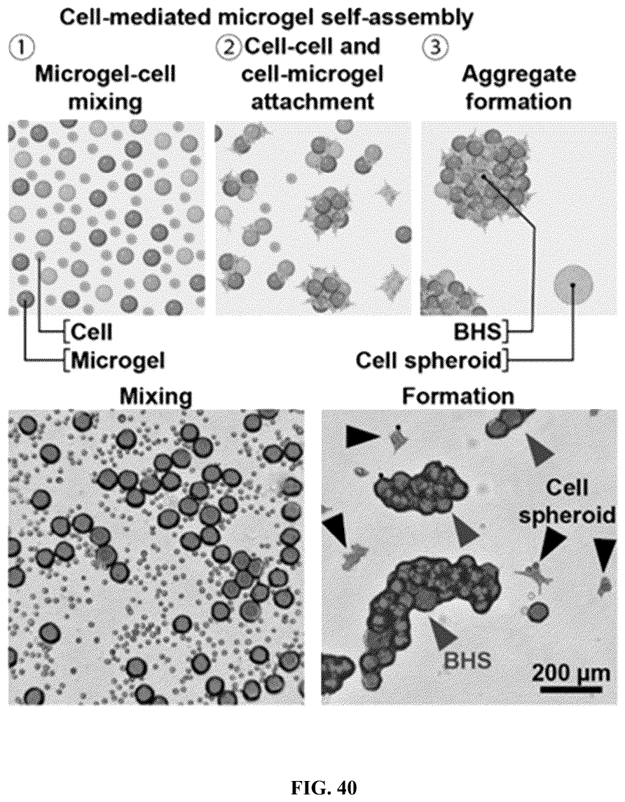

In some embodiments, the microgels may be combined with adherent cells to form hybrid (e.g., cell-microgel) aggregates. In particular, referring to FIGS. 39 and 40, the cells may serve as assembly engines and migrate/adhere to the microgels such that a self-assembly process is initiated and aggregates are formed. In particular, microgels may be significantly larger (≥5 times) than the cells such that porous aggregates are formed. Porous aggregates may enhance molecular diffusion and improve cell viability. Such aggregates may be scalable and be used to self-assemble large-scale physiologically relevant tissue models in vitro.

In some embodiments, the porous aggregates may have a void fraction between 3 and 30%.

In some embodiments, the microgels may be combined with cells on a planar non-adhesive substrate to form the hybrid aggregates. The substrate may prompt cell-cell and cell-microgel interactions.

In some embodiments, combining the cells and microgels may initiate a three step assembly process: (i) initial mixing of cells and microgels, (ii) early-stage attachment of cells to microgels because of the adhesive moieties on the polymer(s), and (iii) the formation of two distinct aggregate types (e.g., hybrid aggregates, and cell aggregates).

In some embodiments, the hybrid aggregates may be crosslinked to form a scaffold. For example, the scaffold may be formed via free radical photopolymerization of the hybrid aggregates.

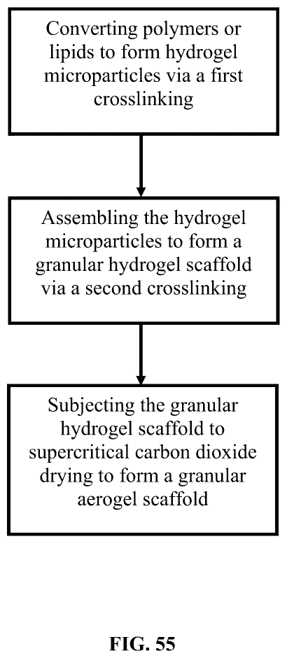

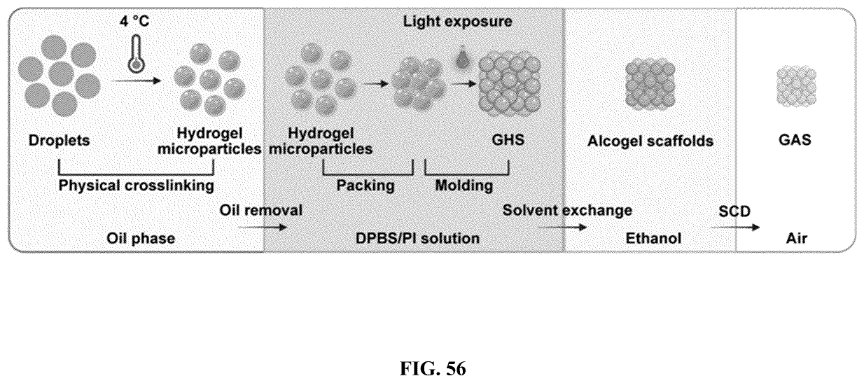

Embodiments relate to granular aerogel scaffolds, which may be formed from the granular hydrogel scaffolds described above. In particular, referring to FIG. 55 and FIG. 56, granular aerogel scaffolds may be formed from jamming (e.g., packing) hydrogel microparticles, followed by crosslinking the hydrogel microparticles through covalent and/or noncovalent intermediate bond formation to yield granular hydrogel scaffolds. As with granular hydrogel scaffolds, the hydrogel microparticles may also serve as the building blocks of the granular aerogel scaffolds. However, after producing the granular hydrogel scaffolds, the scaffolds may be further subjected to supercritical carbon dioxide drying to yield the granular aerogel scaffolds.

The supercritical carbon dioxide drying may preserve the structure of granular hydrogel scaffolds while also providing the advantages of ultralight granular aerogel scaffolds.

In one exemplary method of drying the granular hydrogel scaffolds, after crosslinking (e.g., chemical crosslinking) the microparticles to yield granular hydrogel scaffolds, the aqueous phase may be replaced with an alcohol (such as ethanol) to form an alcogel, followed by supercritical drying to yield granular aerogel scaffolds as the final product. Unlike other drying techniques, such as freeze drying and ambient pressure drying wherein the heterogenous pore size distribution and structural collapse are inevitable, the supercritical drying preserves microparticles and scaffold structural integrity.

Without wishing to be bound by theory, it is contemplated that to preserve the pore structure of hydrogels, supercritical drying may be carried out within the single-phase region of solvent/CO₂ phase diagram at a constant temperature, mitigating the disruptive effects of capillary forces at liquid-vapor interfaces within the pores. Therefore, it may be important that the solvent is miscible with CO₂ at the operating pressure of critical point dryer. A solvent exchange step may therefore be necessary, wherein water in granular hydrogel scaffolds is replaced with ethanol to form an alcogel. Usually, the operating temperature is set slightly above the critical temperature of pure CO₂. Following solvent extraction at the specified pressure, a gradual isothermal depressurization process yields the desired aerogel product.

The granular aerogel scaffolds may have a tunable pore microarchitecture. In particular, controlling and varying the size of the hydrogel microparticles enables regulation of scaffold pore features and properties.

In some embodiments, the compressive modulus of the scaffold may be controlled by controlling the size of the hydrogel microparticles. For example, the compressive modulus of the scaffold may be increased by increasing the size of the hydrogel microparticles. Without wishing to be bound by theory, it is contemplated that this may be a result of more microparticle deformation and less microparticle-microparticle contact (e.g., smaller necks).

Importantly, we have also found that the granular aerogel scaffolds may exhibit similar features and properties as never-dried granular hydrogel scaffold counterparts using the same hydrogel microparticles. For example, rehydration yielded porous aerogel scaffolds that had dynamic and compressive moduli, as well as pore features e.g., (equivalent median pore size and VVF) similar to never-dried granular hydrogel scaffold counterparts fabricated using the same hydrogel microparticles. Accordingly, the granular aerogel scaffolds may recuperate original pore features after rehydration and support cellular activities.

Additionally, we have found that granular aerogel scaffolds support cell migration when seeded from the bottom, thus demonstrating significant counter-gravity capillary-driven cell drawing. Specifically, the cell drawing length may be controlled by controlling the size of the hydrogel microparticles, as scaffolds with larger hydrogel microparticle sizes may exhibit increased cell drawing lengths.

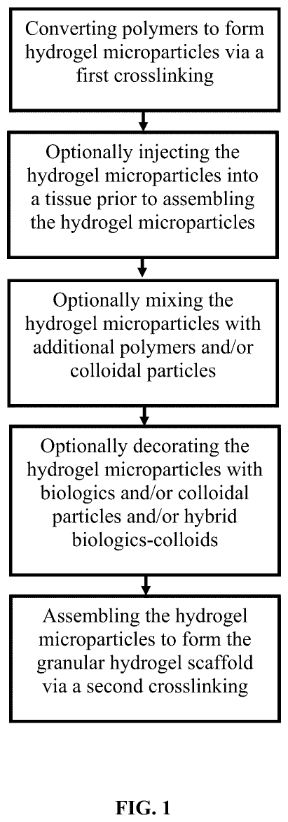

Referring to FIG. 1, embodiments may relate to a method of forming a granular hydrogel scaffold. The method may comprise converting polymers to form hydrogel microparticles (HMP) via crosslinking and assembling the HMP to form scaffolds, such as via free radical photopolymerization or non-light-mediated crosslinking.

In some embodiments, the HMP are injected to an injection site within tissue. In such embodiments, the HMP may assemble to form the scaffolds after injection into the tissue via non-light-mediated crosslinking. It is contemplated that this step of crosslinking may be based on crosslinking of functional groups not used to crosslink the polymer to form the HMP, such as amines, using enzymes, dynamic covalent bond formation, or any other suitable crosslinking technique.

It is further contemplated that the HMP may assemble to form scaffolds after mixing with another polymer and/or colloidal particles. It is contemplated that this polymer may be aldehyde-modified carbohydrates and/or proteoglycans, including hyaluronic acid, protein and/or polymers in the extracellular matrix of native tissues, or another other suitable polymer that may form a hybridized scaffolds.

It is further contemplated that the HMP may assemble to form scaffolds after being decorated with biologics and/or colloidal particles and/or hybrid biologics-colloids. The surface of the HMP may be coated with any biologics and/or the biologics may be encapsulated in the HMP. The biologics may be biomolecules, growth factors (e.g., of hematopoietic growth factors, EGF, FGF, NGF, PDGF, VEGF, IGF, GMCSF, GCSF, TGF, Erythropieitn, TPO, BMP, HGF, GDF, Neurotrophins, MSF, SGF, and GDF and any other growth factors or biomacromolecules), cytokines, enzymatically modified DNA, drugs, peptides, or any combination thereof, or any other suitable biologics that may form scaffolds with enhanced bioactivity (e.g., a bioactive scaffolds). In exemplary embodiments, the biologics may be loaded (i.e., conjugated) to, attached on the surface of, or hybridized with nanocarriers bearing crosslinkable functional groups (e.g., vinyl groups). In exemplary embodiments, the biologics (e.g., the growth factors) are physically and/or chemically attached to colloids (e.g., heparin nanoparticles).

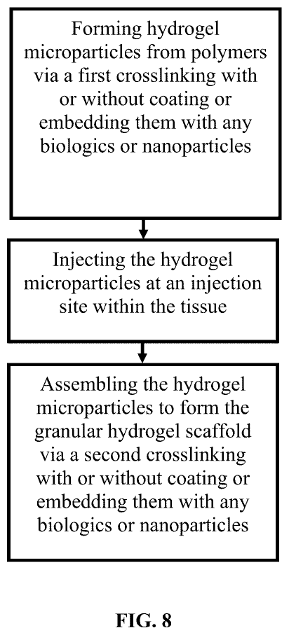

Referring to FIG. 8, embodiments relate to a method of regenerating tissue, inducing vascularization, inducing axonogenesis, and/or improving tissue function via a granular hydrogel scaffold. The method comprises forming HMP derived from polymers via crosslinking, injecting the HMP at an injection site within the tissue, and assembling the HMP to form the scaffolds via non-light-mediated crosslinking. It is contemplated that the scaffolds may be configured to mimic the physiochemical and/or biological characteristics of the tissue. In particular, the scaffolds may be configured to mimic the stiffness of the tissue.

In exemplary embodiments, the tissue is selected from the group consisting of soft and/or hard tissues, including nervous tissue (brain, spinal cord, nerves), endothelial tissue, epithelial tissue (skin, GI tract), muscle tissue (cardiac muscle, smooth muscle, skeletal muscle), and/or connective tissue (fat, bone, tendon, cartilage).

### EXAMPLES

### Example 1

### Materials

Gelatin type A (Sigma-Aldrich, USA), Dulbecco's phosphate buffered saline (DPBS) (Sigma-Aldrich, USA), methacrylic anhydride (Sigma-Aldrich, USA), and dialysis membranes (cutoff M_{w}=12-14 kDa, Spectrumlabs, USA) were used for GelMA synthesis. Lithium phenyl-2,4,6-trimethylbenzoylphosphinate (LAP) (Allevi, USA), (4-(2-hydrozyethyl)-1-piperazineethanesulfonic acid (HEPES) (Gibco, USA), Picosurf (Sphere Fluidics Inc, UK), Novec 7500 (3M, USA), and perfluorooctanol (PFO) (Sigma-Aldrich, USA) were used for GelMA HMP fabrication. Ca²⁺ (Thermo scientific, USA), thrombin (EMD Millipore, USA), and Factor XIII (FXIII) (EMD Millipore, USA) were used for the assembly of GelMA HMP to form GHS. Fluorescein isothiocyanate (FITC) (Sigma-Aldrich, USA) was used for labeling the void spaces of GelMA GHS.

### Methods—GelMA Synthesis

GelMA has been synthesized in the following manner. Briefly, 10% w/v gelatin type A was dissolved in 50° C. DPBS and reacted with methacrylic anhydride 1.25% (v/v) for 2 h. The reaction was stopped by adding DPBS (twice the reaction volume). The GelMA solution was dialyzed against 40° C. miliQ water using dialysis membranes (cutoff M_{w}=12-14 kDa) for 7 days. The GelMA solution was then filtered and frozen for 3 days at −80° C., followed by freeze-drying at 0.12 mbar. The lyophilized GelMA was stored at 2-8° C. until further use.

### Methods—GelMA Stiffness Optimization

Lyophilized GelMA was dissolved in the HEPES buffer (25 mM, pH=7.2-7.4) containing LAP (0.1% w/v). The final concentration of GelMA solution was 1, 1.5, 2, or 3% w/v. The GelMA solutions were transferred to a mold and stored at 2° C. overnight to physically crosslink, followed by UV light (wavelength=395 nm) exposure at an intensity of 15 mW cm⁻² for 30 or 60 s. The photocrosslinked GelMA was punched with an 8 mm biopsy puncher and transferred to a rheometer (TA instrument, USA) to perform frequency sweep tests at a strain rate of 0.1% and angular frequencies of 0.1 to 100 rad s⁻¹. As a proof-of-concept, the average value of photocrosslinked GelMA storage modulus (G′) at 1 rad s⁻¹ was assessed to optimize the GelMA stiffness/concentration for brain studies. This disclosure is valid for any concentration of GelMA or any other protein/peptide/biopolymer.

### Methods—GelMA HMP Fabrication and Stabilization

The GelMA droplet formation may be conducted using a step emulsification or any other device or bulk emulsification. Briefly, a GelMA solution at the optimum polymer concentration that would yield the target tissue stiffness after crosslinking (e.g., 1.5% w/v to mimic brain) was dissolved in the HEPES buffer containing LAP (0.1% w/v). To form GelMA droplets in an oil phase, the GelMA solution was flowed into a microfluidic device as a dispersed aqueous phase, and an oil containing Picosurf 2% (v/v) in Novec 7500 was used as the continuous phase. The GelMA droplets were stored at 2-4° C. to undergo physical crosslinking.

After collecting the GelMA droplets, the emulsion was broken using a PFO solution in Novec 7500 oil (20% w/v). In addition, the microgels were mixed and centrifuged with the solution of 0.1% (w/v) LAP in HEPES to completely remove the oil. The washed microgels were spun down and suspended in a solution of 0.1% (w/v) LAP in HEPES at a concentration of 10% (v/v). Before photocrosslinking, the microgels were always maintained at 4° C. The diluted microgels were placed on a stirrer, and vigorously stirred while being exposed to the UV light (wavelength=395 nm, GearLight, USA) for 60 s at an intensity of 15 mW/cm². The UV exposure initiates the vinyl group-enabled covalent bond formation of GelMA. In this process, other types of crosslinkers, such as other photoinitiators and/or chemical agents (dithiothreitol, DTT) may also be used to stabilize the individual microgels. If the microgels are not stabilized, they immediately dissolve at 37° C. As the GelMA bears vinyl groups, thiol-based crosslinkers, such as DTT, can enable microgel stabilization via the Michael-type reaction between the vinyl and thiol groups. The GelMA within the individual HMP can also be crosslinked using click chemistry by modifying the GelMA carboxyl groups with pendant norbornene and tetrazine. Finally, the crosslinked microgels were stored in HEPES containing 10 mM of Ca²⁺. To assess the stability of microgels, they were transferred to an incubator and imaged over time at 37° C. At timepoints of 0, 0.5, 1, 3, and 24 h, the microgels were imaged using a brightfield microscope. The microgel diameter was analyzed using a custom-written MATLAB code identifying the HMP border. Thermally stable microgels did not undergo significant size change over time.

### Methods—GelMA HMP Modification

To enhance the bioactivity of GHS, embodiments enable the direct modification of GelMA HMP with biomolecules, growth factors (such as VEGF, SGF, and BMP), cytokines, enzymatically modified DNA, drugs, and/or peptides. These modifications can be conducted via GelMA HMP surface modification or encapsulation. In addition, the biologics, such as VEGF, can be loaded/conjugated to nanocarriers, such as heparin nanoparticles (nanoheparin, nH) bearing crosslinkable functional groups, such as vinyl groups. In this case, the growth factor-loaded nH can be directly conjugated to the surface of GelMA HMP through chemical (e.g., covalent) bond formation. The GelMA HMP surface may be coated with any biologics and/or the biologics may be encapsulated in the GelMA HMP. The modified HMP will remain annealable and form composite/nanocomposite GHS, similar to the unmodified HMP.

### Methods—GelMA GHS Formation

The individually crosslinked GelMA HMP were incubated in the HEPES buffer containing 10 mM of Ca²⁺ at room temperature, followed by packing at 14,000 rpm for 5 min. After centrifugation, the supernatant was removed and the excess water among the microgels was removed using a Kimwipe. The packed microgels were aliquoted to microcentrifuge tubes, each of which containing 88 μL of packed GelMA HMP. A solution of thrombin with a concentration of 2 U/mL in HEPES containing Ca²⁺ (10 mM) and a solution of FXIII with a concentration of 10 U/mL in HEPES containing Ca²⁺ (10 mM) were added to two separate aliquoted HMP tubes. Each GelMA HMP aliquot was well mixed with their respective biomacromolecule solutions (FXIII or thrombin), followed by pulse centrifugation. Then, the microgel assembly (GHS formation) was initiated by mixing an equal volume of thrombin-containing microgels and FXIII-containing microgels. Upon mixing, thrombin and calcium activate FXIII, yielding FXIIIa. The two microgel suspensions were mixed using a positive displacement pipette (Gilson, USA). The mixture was spun down at 14,000 rpm, and the supernatant was removed. The mixed microgel was transferred to a mold using a positive displacement pipet and incubated at 37° C. to initiate the FXIIIa-mediated formation of glutamyl-lysine bonds among HMP. In this system, the lysine and glutamine peptides were used to form the glutamyl-lysine bonds with the enzymatic reaction. In addition, based on the target tissue and application, other types of crosslinkers, such as glutaraldehyde, can perform a Schiff-base reaction between lysine and aldehyde groups. GelMA GHS may also be hybridized with other polymers, such as those in the extracellular matrix (ECM) of native tissues. As an example, polysaccharides such as hyaluronic acid (HA) or alginate, were modified with the aldehyde groups to form a Schiff base with protein HMP, such as GelMA. This is an important aspect of the disclosed subject matter, eliminating the necessity of using enzymes, such as thrombin or FXIII, which are expensive, easy to degrade, and often bioactive. Such mechanism can be generalized to other peptides and crosslinkers.

### Methods—GelMA GHS Pore Characterization

To analyze the interconnected void spaces of GelMA GHS, the assembled scaffolds were assessed after 90 min of enzymatic assembly at 37° C. They were incubated with a FITC (Mw=0.5 MDa) solution (15 mM) for 5 min. The labelled scaffold was imaged using a fluorescence microscope (Leica DMI8, Germany). For each sample (at least 3 per condition), the z-stack images were acquired to analyze at least 3 layers of packed microgels within the scaffold. To analyze the scaffold porosity, the void fraction was calculated by adjusting the threshold of images using the Leica LAS X software. In addition, the median pore diameter of scaffolds was calculated by analyzing the equivalent area of individual pores using a custom-written MATLAB code (MATLAB 2021, USA).

### Methods—Mechanical Characterization of GelMA GHS

The assembled GHS (height ˜1 mm) after 90 min of incubation at 37° C. were incubated in room temperature HEPES for 1 h. The scaffolds were punched with 8 mm biopsy puncher, placed on the Instron 5943 (Norwood, MA, USA) lower plate, and underwent compression while the force was measured using a 10 N load cell. The force-displacement data were converted to stress-strain curves. The compression tests were performed at a displacement rate of 1 mm min⁻¹. The compressive modulus of GHS was calculated based on the linear stress-strain region at strain ˜0.05-0.15 mm mm⁻¹. At least 5 scaffolds per condition were analyzed for mechanical characterizations.

### Methods—Statistical Analysis

The one-way analysis of variance (ANOVA) was performed for statistical analysis, and statistically significant differences were identified when p-values were lower than 0.05 (*p<0.05), 0.01 (**p<0.01), 0.001 (***p<0.001), and 0.0001 (****p<0.0001).

### Results and Discussion

### GelMA GHS Formation

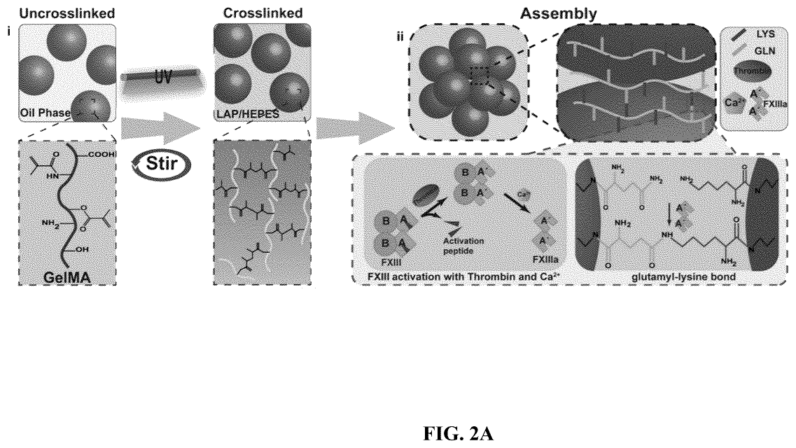

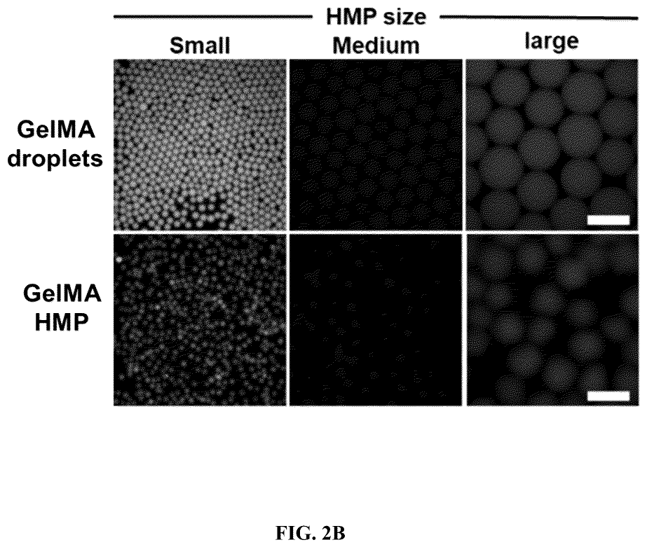

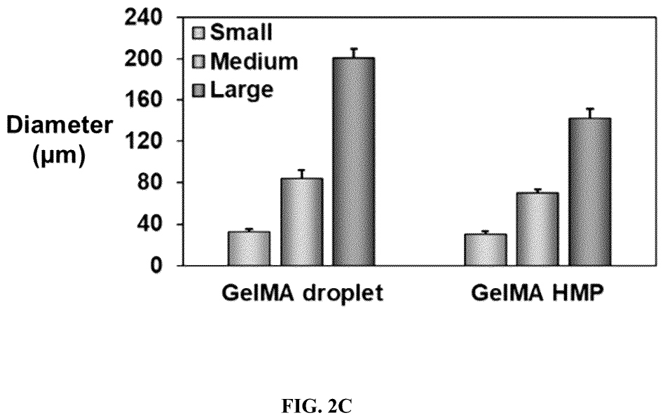

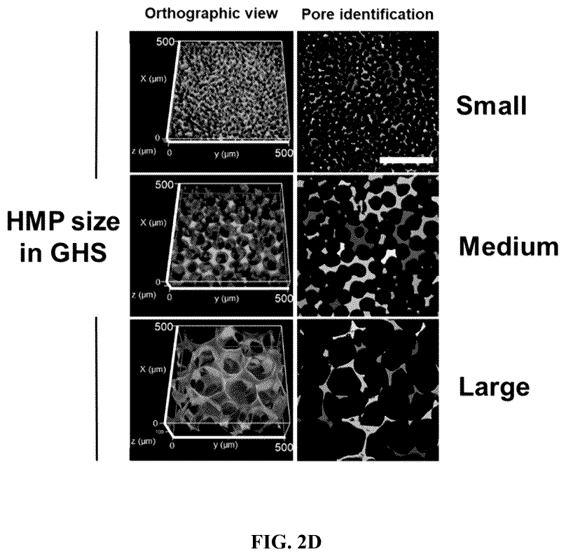

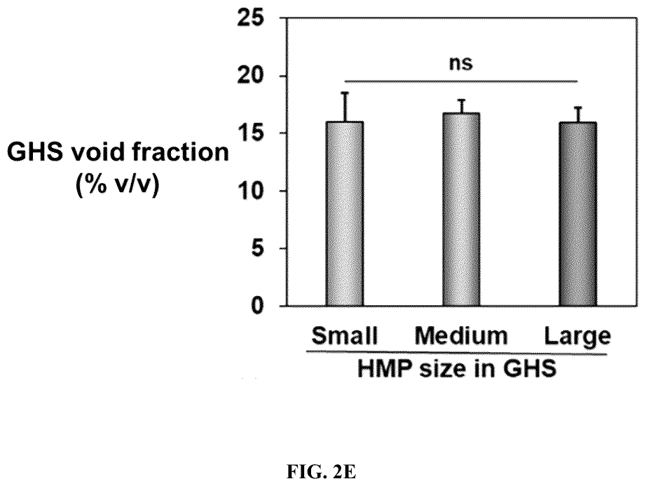

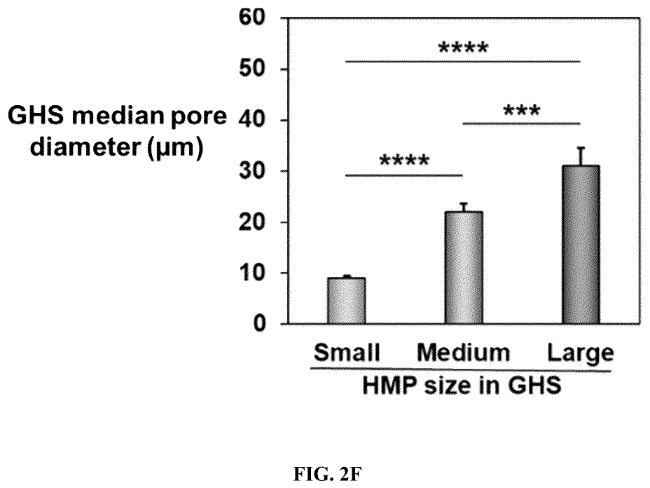

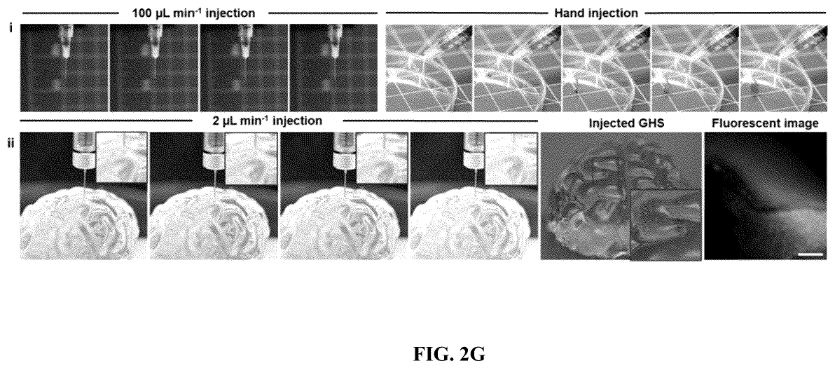

A multi-step procedure to first chemically stabilize HMP and then assemble them to form GHS is provided. GelMA HMP are first physically crosslinked at 2-4° C., followed by UV light (e.g., wavelength ˜395 nm and intensity ˜15 mW cm⁻² for 60 s) exposure in a dilute suspension of HEPES, containing LAP (0.1%, w/v) at 4° C. Throughout the UV exposure process, GelMA precursor within each HMP is chemically crosslinked via free radical polymerization, as schematically shown in FIG. 2A. GelMA contains various peptides, such as lysine (Lys) and glutamine (Gln). The biocompatible assembly of UV crosslinked GelMA HMP to form GelMA GHS is conducted via the enzymatic formation of glutamyl-lysine bonds. FXIII, also known as fibrin stabilizing factor, is a zymogen (an inactive material that is converted to an enzyme upon activation) found in human blood. Thrombin can activate FXIII to form activated FXIII (FXIIIa), which is an enzyme responsible for the crosslinking of fibrin during the blood coagulation cascade. FXIIIa catalyzes the formation of ε-(γ-glutamyl) lysine isopeptide bonds between the ε-amino groups of Lys residues (K peptide, donor) and γ-carboxamide groups of Gln residues (Q peptide, acceptor) of gelatin, similar to those of fibrin monomers.

FIG. 2A schematically presents the mechanism of GelMA GHS formation via the FXIIIa mediated glutamyl-lysine bond formation among the individual HMP. Accordingly, the GelMA HMP undergoes microgel-microgel assembly, catalyzed by the FXIIIa at 37° C., forming GelMA GHS.

The pore characteristics of GHS directly regulate the scaffold-cell interactions, cell recruitment, foreign body response, and tissue regeneration. Accordingly, it is important to tailor the void space within GHS. The pore features of GHS are tuned by varying the size of HMP building blocks, as the pore size is regulated by the void space among the microgels. To engineer the pore size of GHS, various GelMA HMPs were fabricated with an average diameter of 31.9±3.2, 85.5±6.0, or 164.8±14.6 μm, labeled as small, medium, or large, respectively (see FIGS. 2B and 2C).

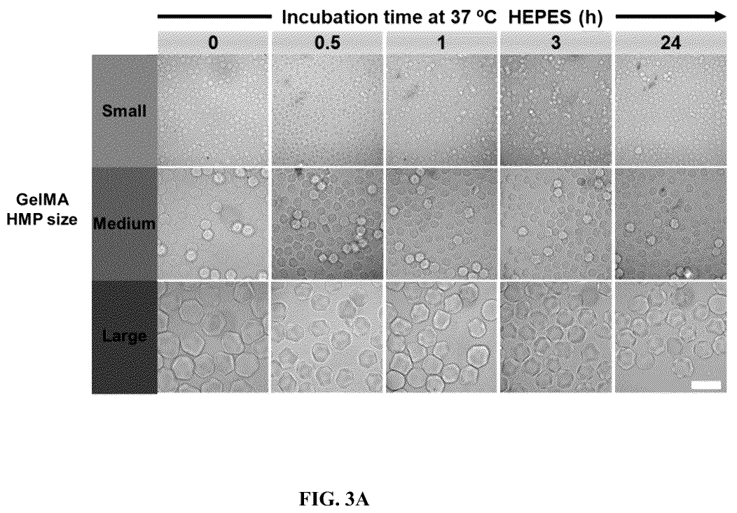

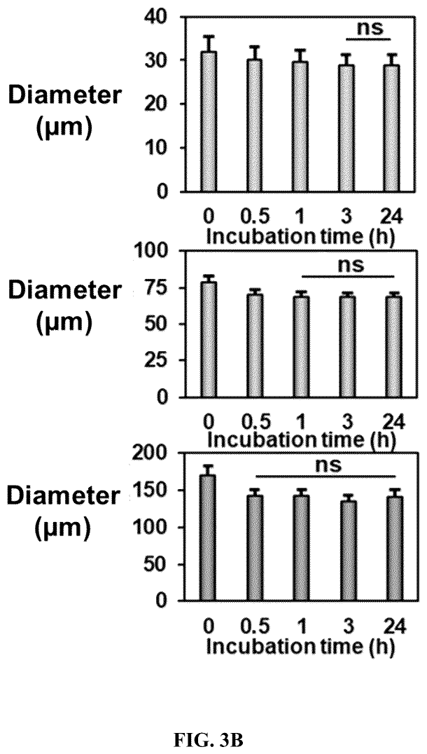

FIGS. 3A and 3B shows the size change of photocrosslinked GelMA HMP at 37° C. As can be seen in these figures, the photocrosslinked GelMA HMP are stable at the physiological temperature (before assembly). Finally, the GelMA GHS were fabricated by mixing the GelMA HMP with the FXIIIa, followed by packing and incubation at 37° C. for 90 min.

To demonstrate the interconnectivity of pores and analyze the pore features of GelMA GHS, the scaffolds were incubated with the FITC-dextran fluorescent dye. FIG. 2D shows the fluorescently labeled GelMA GHS, fabricated from three groups of GelMA HMP (small, medium, and large) as well as the identified pores using a custom-written MATLAB code.

FIG. 2E shows the analysis of GelMA GHS void fraction, which suggests that the void fraction is independent of microgel size, and the average void fraction is ˜16.0±2.4% (v/v). In addition, the median pore size of GelMA GHS was measured at a z-stack wherein the microgels have the largest contact with each other. The images were analyzed using the MATLAB code to detect the area of individual pores and calculate the equivalent pore diameter. As presented in FIG. 2E, the median pore diameter of GelMA GHS fabricated from the small, medium, and large HMP is ˜8.9±0.4, 21.9±1.6, and 30.9±3.5 μm, respectively. These results show that although the void fraction is similar in all three groups of GHS as a result of spherical granules, the pore size significantly increases by increasing the microgel size.

FIG. 2G shows the injectability of packed GelMA HMP through 30G needles using a syringe pump or by hand, mimicking the brain injection process to facilitate tissue regeneration after stroke. FIG. 2G also presents the HMP injection via a 30G needle (Small Hub RN Needle, Hamilton, USA) connected to a 5 μL syringe (Hamilton, USA) using a syringe pump at a rate of 2 μL min⁻¹. As can be seen in FIG. 2G, the GelMA HMP is injectable via the clinically relevant needles, enabling scaffold formation in a lesion after injection.

### Mechanical Characterization of GelMA HMP and GHS

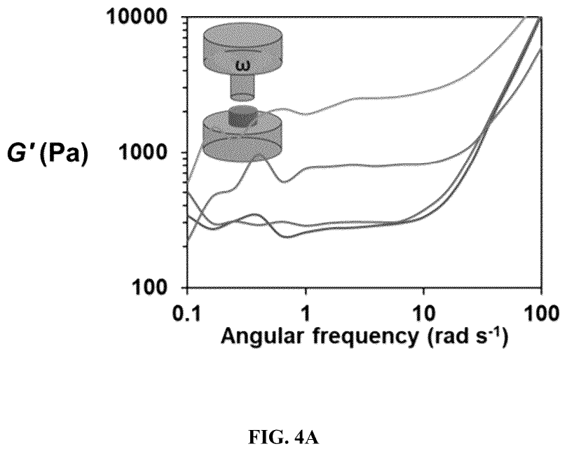

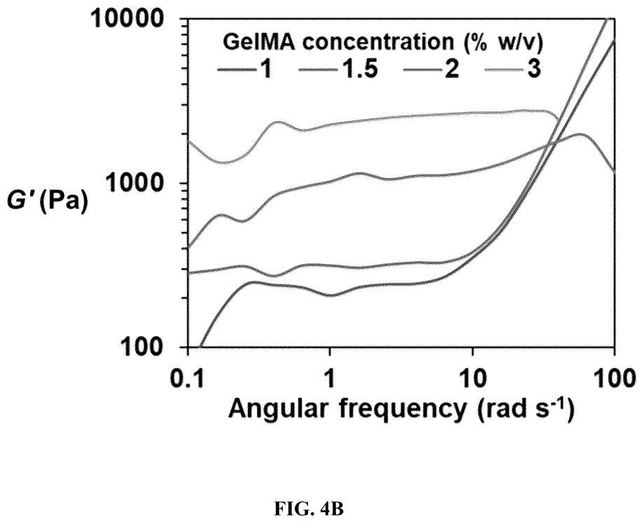

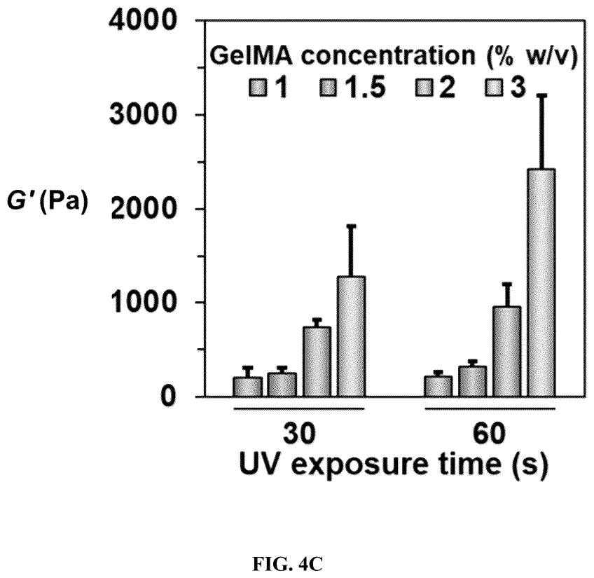

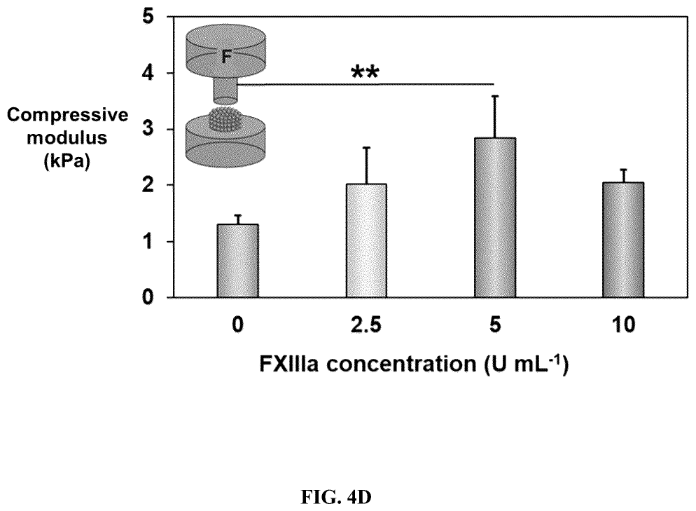

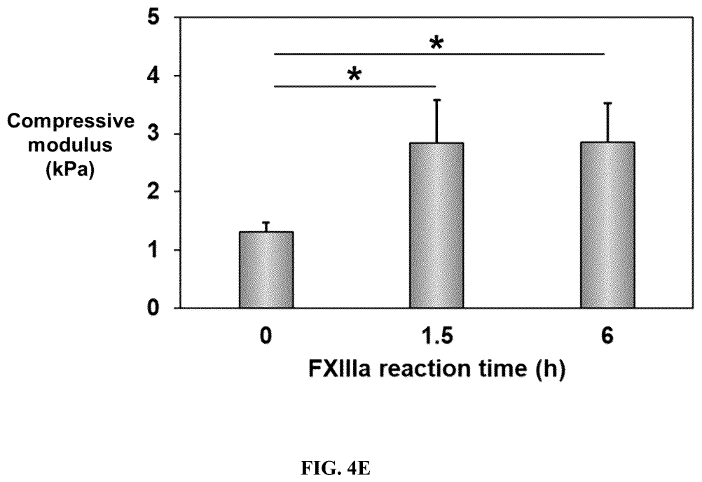

The mechanical properties of GelMA should be optimized to mimic target tissues. As an example, to match the stiffness of brain tissue, the stiffness of GelMA was tailored via changing the GelMA concentration and photocrosslinking time. Varying GelMA concentrations (1, 1.5, 2, or 3% w/v in HEPES, containing 0.1% LAP) were used to prepare physically crosslinked bulk samples maintained at 2-4° C. overnight, followed by UV light exposure for 30 s or 60 s at an intensity of 15 mW cm⁻². The representative storage modulus graphs are shown in FIGS. 4A and 4B for these exposure times, respectively.

FIG. 4C presents the average and standard deviation of bulk GelMA storage modulus at varying GelMA concentration and photocrosslinking times. The higher the biopolymer concentration or UV exposure time, the higher the storage modulus within the experimental range. The target average value of storage modulus at a frequency of 1 rad s⁻¹ is the native rat brain modulus of ˜330 Pa. The average storage modulus of the GelMA with a concentration of 1.5% (w/v) and the UV curing time of 60 s was around 325 Pa, which has a good agreement with the native brain tissue (FIG. 4C). Note that the stiffness of the bulk samples represents the stiffness of individual GelMA HMP.

The HMP assembly process was initiated by mixing the GelMA HMP with FXIIIa, followed by incubation at 37° C. To determine the stiffness of GelMA GHS, compression tests were performed on the GHS. The concentration of the FXIIIa was optimized based on the compressive modulus of assembled scaffolds, compared with packed unassembled ones (the control group).

FIG. 4D shows the compressive modulus of unassembled (FXIIIa concentration=0) and assembled GHS with the FXIIIa concentration of 2.5, 5, and 10 U mL⁻¹. The average compressive modulus of unassembled GHS (i.e., packed HMP) was around 1.31±0.15 kPa, and that of assembled GHS was 2.02±0.64, 2.83±0.74, and 2.05±0.22 kPa for the FXIIIa concentrations of 2.5, 5, and 10 U mL⁻¹, respectively. Accordingly, FXIIIa with a concentration of 5 U mL⁻¹, obtained from activating FXIII using thrombin (1 U mL⁻¹), resulted in GHS stiffness that was significantly higher than the control group (no FXIII), which will be used as the optimum enzyme concentrations for the GHS formation (see FIG. 4D).

FIG. 4E shows the compressive modulus of GelMA GHS formed at two different FXIIIa reaction times (1.5 and 6 h) compared with the packed HMP that did not undergo the FXIIIa reaction. The compressive modulus of assembled GHS at the FXIIIa concentration of 5 U mL⁻¹ was around 2.83±0.74 or 2.36±0.96 kPa for the GHS incubation time of 1.5 or 6 h at 37° C., respectively. These results show that the scaffold stiffness reaches a plateau after 1.5 h of incubation at 37° C. Therefore, the scaffolds are less likely to undergo further stiffening after 1.5 h post injection in tissues, such as brain.

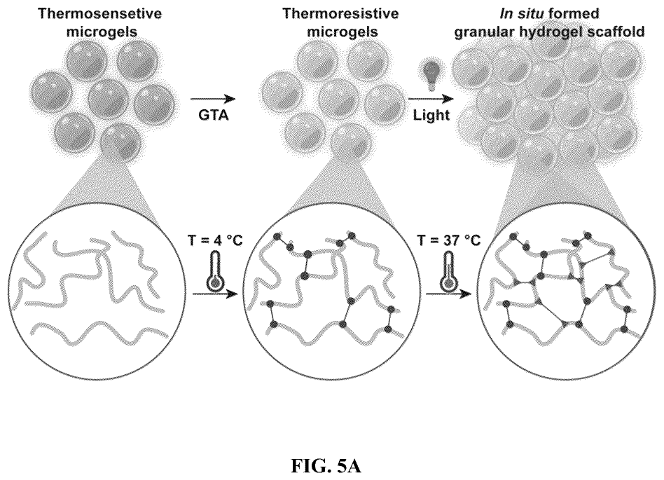

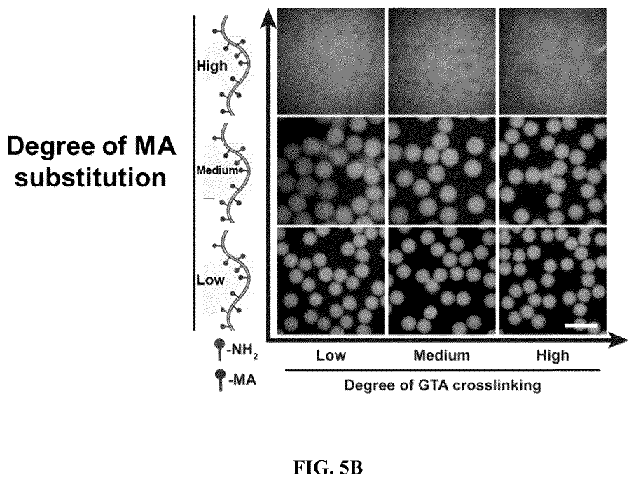

FIGS. 5A-C show in situ formation of GHS from thermoresistive GelMA microgels. FIG. 5A shows that GelMA HMP are thermoresponsive and unable to form a scaffold at body temperature (e.g., 37° C.). To overcome this limitation, the GelMA HMP was crosslinked via a Schiff-base reaction to produce thermoresistant microgels. These thermoresistive microgels served as building blocks for the in situ fabrication of GHS via radical photopolymerization.

FIG. 5B shows that GelMA was synthesized using three different degrees of substitutions (DoS) with high (˜70%), medium (˜40%) and low (˜10%), which affected the availability of primary amine groups for Schiff-base crosslinking using glutaraldehyde (GTA). High DoS resulted in insufficient primary amine groups, making the thermoresistive microgel unstable. In contrast, medium and low DoS resulted in stable thermoresistive microgels.

FIG. 5C shows thermoresistive microgels from medium and low GelMA were scaffolded to form a mechanically stable GHS.

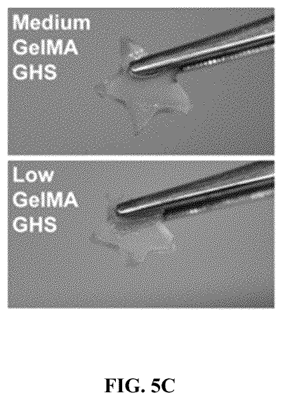

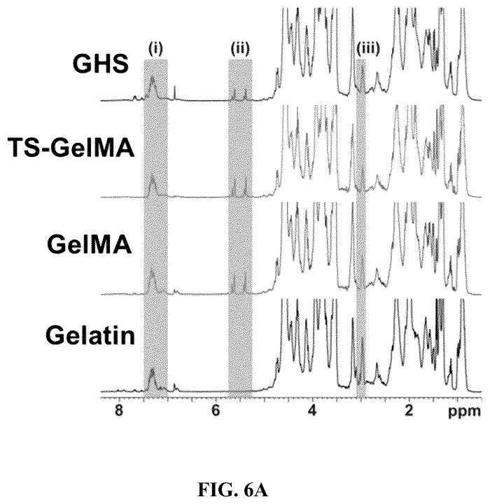

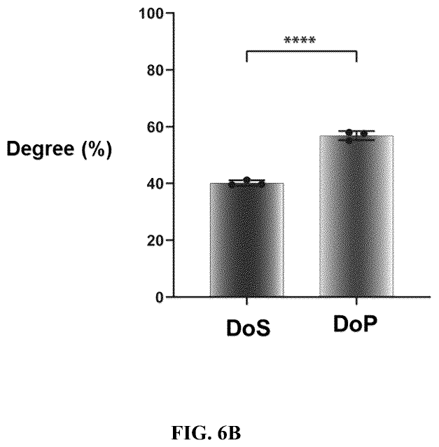

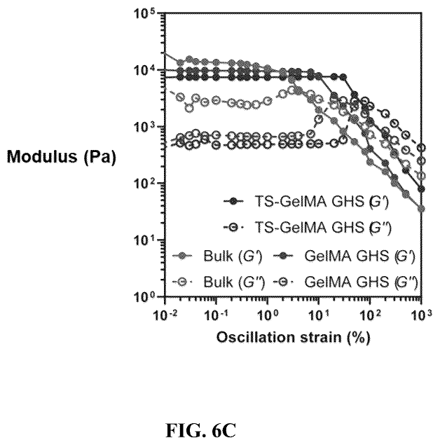

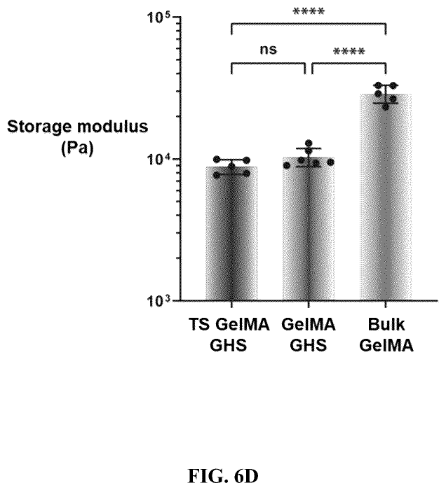

FIGS. 6A-D show material and mechanical characterization of GHS made from thermoresistant microgels. FIG. 6A shows ¹H NMR spectrometry of pure gelatin microgel, uncrosslinked GelMA microgel (with medium DoS), thermoresistant GelMA HMP, and photocrosslinked GHS composed of thermoresistant microgels. The peaks for aromatic acids (i) serve as the reference in all the groups. The presence of vinyl groups (ii) and a decrease in lysine protons (iii) were also observed.

FIG. 6B shows oscillatory strain sweep at constant frequency of 1 rad/s performed on in situ fabricated scaffolds: GHS from thermoresistant GelMA microgels (TS GelMA GHS), GHS from thermoresponsive GelMA microgels (GelMA GHS), and conventional (bulk) GelMA scaffolds.

FIG. 6C shows dynamic moduli versus oscillation strain for different scaffolds at a constant frequency of 1 rad/s, showing the storage and loss moduli of TS GelMA GHS, GelMA GHS, and conventional (bulk) GelMA scaffolds.

FIG. 6D shows the storage modulus measured at 1 rad/s and 0.1% strain. No significant difference was observed between the TS GelMA GHS and GelMA GHS, but bulk GelMA had significantly higher stiffness.

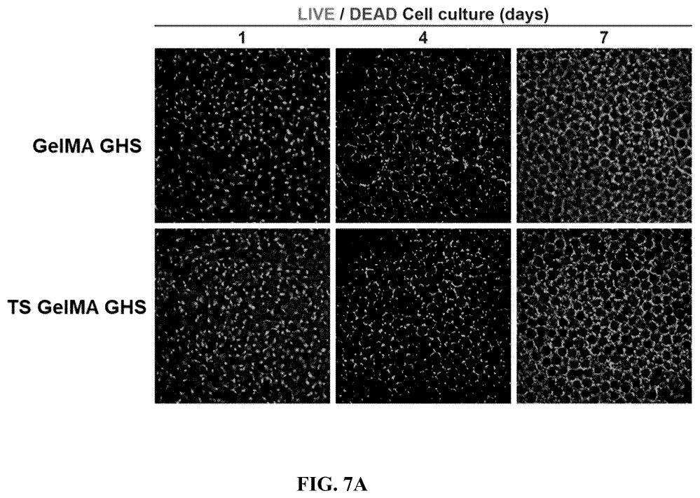

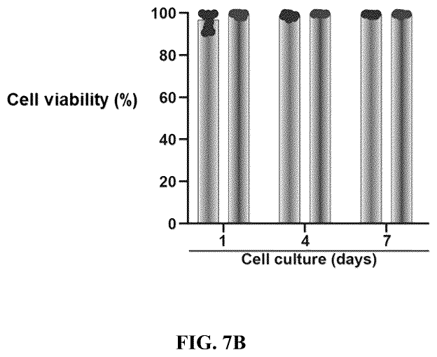

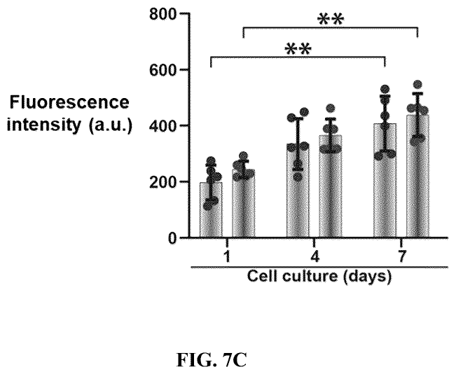

FIGS. 7A-C show biocompatibility assessment of GHS made from thermoresistant microgels. FIG. 7A shows that NIH-3T3 murine fibroblast cells were mixed with thermoresponsive or thermoresistant microgels, then photocrosslinked to form GelMA GHS or TS GelMA GHS, respectively. Fluorescent microscopy using a combination of green (representing live cells) and red (representing dead cells) dyes was performed over a period of 7 days, demonstrating cell proliferation and viability.

FIG. 7B shows the results if cell viability percentage indicated a consistently high viability rate (˜100%) for all GHS samples.

### Example 2: Effect of Porous Microgels

### Materials

Bovine serum albumin (BSA), citrate buffer (pH=6.0), dimethyl sulfoxide (DMSO), Dulbecco's phosphate buffered saline (DPBS, powder), Fluoroshield™ with 4′,6-diamidino-2-phenylindole (DAPI), fluorescein isothiocyanate-dextran (FITC-dextran, average molecular weight=2 MDa, equivalent Stokes diameter ˜54 nm), gelatin powder (Type A, ˜300 g Bloom, from porcine skin), lithium phenyl-2,4,6-trimethylbenzoylphosphinate (LAP), methacrylic anhydride, polyethylene glycol (PEG, number average molecular weight=20000), TWEEN® 20, 1H,1H,2H,2H-perfluoro-1-octanol (PFO), and Triton X-100 were purchased from MilliporeSigma, MA, USA. Milli-Q water purification system, used to produce ultrapure water (electrical resistivity ˜18.2 MΩ cm at 25° C.), was provided by Millipore Corporation, MA, USA. Spectra/Por 4 dialysis tubing with 12-14 kDa molecular weight cut-off was purchased from Spectrum Laboratories, NJ, USA. Vacuum filtration unit (pore size=0.20 μm), and VWR® VistaVision™ microscopic slides were purchased from VWR, PA, USA. Novec™ 7500 Engineered Fluid was purchased from 3M, MN, USA. Pico-Surf® (2% w/w in Novec™ 7500) was provided by Sphere Fluidics, Cambridge, UK. Fetal bovine serum (FBS) and antibiotic/antimycotic solution (10,000 U mL-1 penicillin G, 10,000 μg mL-1 streptomycin, 25 μg mL-1 amphotericin B) were supplied by Cytiva, MA, USA. DPBS (liquid), phosphate buffered saline (PBS), Dulbecco's Modified Eagle's Medium (DMEM), and trypsin-ethylenediaminetetraacetic acid (EDTA) solution (0.25%) were provided by Gibco, MA, USA. 24 well non-treated plate, T-75 cell culture flasks, and centrifuge tubes (15 mL and 50 mL) were supplied by Celltreat Scientific Products, MA, USA. 96-well flat clear bottom black polystyrene microplates were purchased from Corning, NY, USA. Live/Dead™ cell imaging kit (488/570) containing calcein acetoxymethyl ester (calcein AM) and BOBO-3 iodide, PrestoBlue™ HS (high sensitivity) cell viability reagent, and Alexa Fluor™ 488 phalloidin were provided by Invitrogen, MA, USA. Xylene substitute, Hematoxylin 560™, Alcoholic Eosin Y 515™, Blue Buffer 8™, and Define® were purchased from Leica Biosystems, IL, USA. Recombinant Alexa Fluor 488 anti-cluster of differentiation 31 (CD31) antibody (ab305267) and Alexa Fluor 647 anti-CD68 antibody (ab305214) were purchased from Abcam, CA, USA. Diamond® white glass charged slides, and snap cap 1.7- and 2-mL microcentrifuge tubes were purchased from Globe Scientific, NJ, USA. Alpha-smooth muscle actin (α-SMA) monoclonal antibody (1A4), Alexa Fluor™ 488, eBioscience™ (53-9760-82), and paraformaldehyde (PFA) were purchased from Thermo Fisher Scientific, MA, USA. Isoflurane was provided by Akorn, Inc., IL, USA. Loxicom® meloxicam solution (5 mg mL¹) was purchased from Norbrook Laboratories, KS, USA. Ethanol (200 proof) was provided by Decon Labs, Inc., PA, USA. Chlorhexidine® scrub was supplied by Aspen Veterinary Resources LTD., CO, USA. Surgipath Paraplast X-tra was provided by Leica Biosystems, Germany. Macrosette® processing/embedding cassettes with cover were supplied by Ted Pella Inc., CA, USA. Acrylic sheets were purchased from Astra Products Inc., NY, USA.

### Methods—GelMA Synthesis

GelMA synthesis was carried out based on published protocols. Briefly, 16 mL of methacrylic anhydride was gradually added to 200 mL of a gelatin solution in DPBS (concentration=10% w/v), while being continuously stirred at 50° C. The reaction was terminated after 2 h through the addition of 400 mL of DPBS at 40° C. Subsequently, the solution underwent dialysis against ultrapure water at 40° C. for 10 days using the dialysis membrane with a molecular weight cutoff of 12-14 kDa. The dialyzed solution was then filtered using a 0.20 μm vacuum filtration system and stored at −80° C. for at least 48 h. The frozen solution underwent lyophilization in a Labconco FreeZone 4.5 L Benchtop freeze dryer (Labconco, MO, USA) at vacuum pressure ˜0.016 mbar until a solid GelMA product was obtained. The degree of methacryloyl substitution for three independently synthesized batches used in this study was 75±1%, 73±2%, and 75±1% (n=3, for each batch), determined according to published protocol.

### Methods—Microgel Fabrication

GelMA solutions (7 or 14% w/v) were prepared by dissolving the lyophilized GelMA polymer in DPBS, containing LAP photoinitiator (0.1% w/v). Additionally, PEG solutions with varying concentrations (3, 4, or 5% w/v) were prepared by dissolving the PEG polymer in DPBS, containing LAP (0.1% w/v). For the GelMA-PEG mixtures, the final precursor polymer solution was created by mixing equal volumes of the GelMA (14% w/v) and PEG solutions. The GelMA (7% w/v)-PEG mixtures or a GelMA solution (7% w/v) formed the dispersed phase, while the continuous phase comprised Novec™ 7500 Engineered Fluid, supplemented with Pico-Surf™ (0.5% v/v). The two phases were introduced into a step emulsification microfluidic device, fabricated based on a published work (adopted from an established protocol) via two syringe pumps (PHD 2000, Harvard Apparatus, MA, USA). The microfluidic droplet fabrication setup was maintained at 37-40° C. using a space heater to prevent GelMA physical gel formation during the droplet fabrication. The resulting droplets were collected in microcentrifuge tubes, placed in water baths at fixed temperatures to induce phase separation. The bath temperature was monitored using a digital J/K type thermometer (TM100, Extech Instruments, NH, USA). Subsequently, the droplet suspensions were stored at 4° C. overnight, leading to the formation of physically crosslinked microgels.

### Methods—Measuring the Cooling Rate of Droplet Suspensions

To evaluate the average cooling rate, the droplet suspension in oil, stored in a 1.7 mL microcentrifuge tube, was initially placed in a water bath at 37° C., marked as the initial temperature (Tᵢ). The droplet suspension was then transferred to another water bath at a fixed temperature (0, 10, or 20° C.), and the suspension temperature was recorded every 5 s until the final suspension temperature (T_{f}) reached the water bath temperature (0, 10, or 20° C.) and remained constant for 15-20 s.

### Methods—Measuring Phase Separation Temperature

To determine the phase separation temperature of varying GelMA-PEG mixtures, polymer solution mixtures containing a fixed GelMA concentration (7% w/v) and varying PEG concentrations (1.5, 2, or 2.5% w/v) were pipetted into pre-warmed (37° C.), laser-cut, disk-shaped reservoirs (diameter=10 mm and height=1 mm) and sandwiched between two microscope slides. The samples were then placed in a temperature-controlled bold line stage top incubator (Okolab, Pozzuoli, Italy) set to 37° C. The temperature was gradually reduced from 37° C. to 25° C. at 1° C. intervals every 30 min. For temperatures below 25° C., the reservoirs were transferred onto a temperature-controlled hotplate, and the temperature was decreased from 25° C. to 21° C. in 1° C. intervals every 30 min. Throughout this process, at each interval, the samples were closely monitored and imaged using a DMi8 THUNDER Imager three-dimensional (3D) Cell Culture microscope (Leica Microsystems, Germany). The phase separation temperature was reported as the average of temperatures at which phase-separated patterns in microgels were observable using microscopy images in three independently prepared samples.

### Methods—GHS Formation

The physically crosslinked microgels, phase-separated at varying T_{f} (0 or 20° C.), were washed once with PFO (20% v/v in Novec™ 7500 Engineered Fluid) at ˜4° C., using a 1:1 volume ratio, to remove the surfactant and oil from the suspension. The suspension was then mixed with 200 μL of a LAP solution (0.1% w/v in DPBS), followed by vortexing, centrifugation at 325×g for 15 s, and supernatant removal. This process was repeated twice. The microgels were then vigorously vortexed for 15 s and allowed to sediment after which the supernatant was collected and replaced with a fresh LAP solution (0.1% w/v in DPBS). This process was repeated three times. To maintain the microgels physically crosslinked during the washing steps, a cold-water bath (temperature ˜4° C.) was used. The washed microgel suspension was then centrifuged at 2940×g for 15 s, and the supernatant was removed. The packed microgel suspension was pipetted into laser-cut cylindrical molds with varying dimensions using a positive displacement pipette (Microman E M100E, Gilson, OH, USA). Finally, the molded microgels underwent photocrosslinking via light (UV LED Flood Light, source power=20 W, wavelength=395-405 nm, QUANS, China) exposure for 2 min at an intensity of ˜15 mW cm⁻².

### Methods—Microgel Pore Characterization

The aqueous microgel suspension was centrifuged at 325×g for 15 s, and the supernatant was removed. Subsequently, 100 μL of microgels were mixed with 900 μL of DPBS, containing LAP (0.1% w/v), and exposed to the light (395-405 nm and 15 mW cm⁻²) for 2 min while stirring at 1000 rpm in 2 mL microcentrifuge tube on a magnetic stirrer (Four E's Scientific, China) at 4° C. The individually photocrosslinked microgels were then incubated in 200 μL of FITC-dextran solution (12 μM in DPBS) for 30 min. Z-stacks of FITC-incubated microgels (average height ˜80 μm, ˜160 slices, height increment size ˜0.5 μm) were imaged from the surface to the center of each microgel using a DMi8 THUNDER Imager 3D Cell Culture microscope, equipped with a 25× objective (HC FLUOTAR L 25×/0.95 W VISIR, Leica Microsystems, Germany). Then, 3D images were rendered from the Z-stacked images using the Leica application suite X (LAS X, version 3.7.4.23463). The void fraction and average median pore size of individual microgels were characterized using MATLAB (MATLAB, version R2023b), respectively. The void fraction was determined by summing the area of FITC-labeled pixels and dividing it by the area enclosed by detected edges, which were identified as the last local minimum in the FITC-occupied area before reaching the highest FITC-occupied area. Average median pore size of microgels was determined two-dimensionally (2D) by skeletonizing FITC-labeled areas and obtaining the median of distances from each point along the skeleton to the adjacent unlabeled region for each layer of the Z-stacks. The median pore size of layers was then averaged and reported for each microgel (n>16).

### Methods—GHS Pore Characterization

To assess the GHS void fraction and median pore size, 100 μL of a FITC-dextran solution (12 μM in DPBS) was added on top of the scaffolds (diameter=8 mm and height=0.4 mm), followed by incubation for 30 min at room temperature. The scaffolds were then imaged in 2D at the layer of microgel-microgel contact. GHS median pore size was characterized using MATLAB as described earlier in section 1.7. Void fraction was assessed using a MATLAB code by calculating the ratio of FITC-labeled area to the entire area within the randomly selected regions of interests (ROIs) (n=5 samples). Measuring void fractions using 3D images constructed from Z-stacks was not feasible with the DMi8 THUNDER Imager 3D Cell Culture microscope or the Leica DMi8 laser scanning confocal microscope equipped with STELLARIS 5 White Light Lasers (Leica Microsystems, Germany). The imaging was limited to a certain depth from the glass surface to approximately the center of the microgels, where the highest microgel-microgel contact was observed. Imaging beyond this depth proved impossible, likely because of the porous structure of the microgels, which caused significant light refraction. For void fraction analysis, 2D images were acquired at the depth where the highest microgel-microgel contact was observed, which provided the lowest void fraction in the scaffold. Additionally, the void fraction observed in samples made with nonporous microgels at this depth was consistent with previous observations, where the void fraction of GHS made with microgels of similar size (˜170 μm) was reported to be 20-25%. Median pore size was determined by skeletonizing the FITC-labeled areas in images, wherein maximum microgel-microgel contact was observed, and obtaining the median of distances from each point along the skeleton to the adjacent unlabeled region (n=5 samples).

### Methods—Mechanical Characterization of GHS

To measure the compressive modulus of GHS, cylindrical specimens (diameter=8 mm and height=3 mm) were fabricated and incubated in DPBS at room temperature overnight. The samples were then subjected to a compression test using an Instron mechanical tester (Instron 5943, MA, USA) at a compression rate of 1 mm min⁻¹. The slope of elastic region in the compressive stress-strain curve (within 0.05-0.15 mm mm⁻¹ strain) was measured and reported as the compressive modulus of GHS.

### Methods—Rheological Characterization of GHS

Oscillatory rheological tests were performed on disk-shaped specimens (diameter=8 mm and height=1 mm) using an AR-G2 rheometer (TA instrument, DE, USA), equipped with 8 mm diameter top and 25 mm bottom sandblasted plates. All the fabricated samples were incubated in DPBS at room temperature overnight, followed by conducting the tests at 25° C. The scaffolds remained hydrated during the test via adding a few DPBS droplets around them. The storage and loss moduli were determined through a frequency sweep test, conducted at constant strain ˜0.1% within the linear viscoelastic region (LVR) and frequency ˜0.1-100 rad s⁻¹. The LVR was identified by an amplitude sweep test at strain ˜0.01 to 1000% and a fixed frequency (1 rad s⁻¹).

### Methods—Scanning Electron Microscopy (SEM) Imaging

Ethanol solutions of varying concentrations (30, 50, 60, 70, 80, 90, and 95% v/v) were prepared using ultrapure water. Disk-shaped GHS specimens (diameter=8 mm and height=3 mm) were placed in a Petri dish, containing 10 mL of 30% v/v ethanol, and incubated at room temperature for 10 min. The samples were then sequentially incubated in Petri dishes with increasing ethanol concentrations at 10-min intervals. After the final incubation in a 95% v/v ethanol solution, the specimens were incubated in 10 mL of 100% ethanol for a 30 min at room temperature. The last step was repeated two more times. Following the final ethanol incubation, the scaffolds underwent supercritical drying using a critical point dryer (CPD300, Leica Microsystems, Germany). To prepare the samples for SEM imaging, they were coated with iridium (thickness ˜5.6 nm) using a low vacuum sputter coater (Leica EM ACE200, Germany). The surface properties of the GHS specimens, after undergoing the supercritical drying process, were visualized using a scanning electron microscope (Quanta 250 ESEM, Thermo Scientific, OR, USA) at an accelerating voltage of 3-5 kV and a beam current of 0.67 nA for scaffolds made with nonporous microgels and 53 pA for scaffolds made with porous microgels.

### Methods—In Vitro Cell Culture

The NIH/3T3 murine fibroblast cells (ATCC, VA, USA) were cultured in DMEM, containing 10% v/v FBS and 1% v/v antibiotics. The culture was maintained in a cell culture incubator (Eppendorf, Hamburg, Germany) under a 5% v/v carbon dioxide (CO₂) atmosphere at 37° C. Cells were passaged once they reached ˜80% confluency by detaching them from T-75 cell culture flasks using a trypsin-EDTA solution (0.25%) and counted using a cell counter (Countess™ II automated cell counter, Thermo Fisher Scientific, MA, USA). To conduct in vitro studies involving GHS, cells were mixed with microgels that were packed at 2940×g for 15 s to obtain a cell density of 4×10³ per μL of packed microgel suspension. Subsequently, cell-microgel mixture was pipetted into cylindrical molds (diameter=6 mm and height=3 mm) using a positive displacement pipette and photocrosslinked via light exposure (395-405 nm and 15 mW cm⁻²) for 2 min.

Methods—Assessing Cell Infiltration into Porous Microgels

To evaluate cell infiltration within individual porous or nonporous microgels, the aqueous microgel suspensions were centrifuged at 325×g for 15 s, followed by supernatant removal. Subsequently, the microgels were mixed at a 1:10 ratio with a LAP solution (0.1% w/v in DPBS) and individually photocrosslinked using light (395-405 nm and 15 mW cm⁻²) for 2 min at 4° C., while being stirred at 1000 rpm to prevent interparticle crosslinking. Photocrosslinked microgels were then centrifuged at 325×g for 15 s, followed by supernatant removal. Afterwards, 200 μL of individually crosslinked, packed microgels were added to a non-treated Petri dish, containing 5 mL of cell suspension in culture media (10⁶ NIH/3T3 murine fibroblast cells per mL). The suspension was then thoroughly mixed via pipetting and maintained under a 5% CO₂ atmosphere at 37° C. in the cell culture incubator. After 48 h, cells were fluorescently labeled through the incubation of cell-microgel suspension in 5 mL of calcein AM solution (1 μM in 10 mL of live cell imaging solution) at room temperature for 30 min. The suspension was then vortexed for 10-15 s, using a Fisherbrand™ variable speed mini vortex mixer (Thermo Fisher Scientific, MA, USA), to disintegrate microgel-cell aggregates, resulting in individual cell-adhered microgels. Imaging was conducted using the DMi8 THUNDER Imager 3D Cell Culture microscope at excitation/emission wavelengths of 470 nm/510 nm to obtain the Z-stacks of single microgels with an average Z-depth of ˜90 μm and a height increment size of 0.5 μm. All the Z-stacks were analyzed in each layer (2D) to obtain the total cell-infiltrated volume in each microgel using MATLAB. Briefly, images were blurred using a Gaussian noise deconvolution and binarized using adaptive thresholding. Afterwards, edges were detected, and the background was masked. The labeled pixels within the unmasked area were summed to find the area occupied by cells for each layer. This area was then multiplied by Z-step size, and the process was repeated over the entire Z-stack for each layer. The reported volume of infiltrated cells was calculated by summing the volume of all layers.

### Methods—Cell Viability Assessment

The live/dead cell viability assay was used to assess the viability of cells cultured in the GHS, fabricated using porous or nonporous microgels. To this end, NIH/3T3 murine fibroblast cells were mixed with microgels to obtain a cell density of 4×10³ per μL of microgels (packed via centrifugation at 2940×g for 15 s). Then, the mixture was pipetted into cylindrical molds (diameter=6 mm and height=3 mm) and photocrosslinked using the light (395-405 nm and 15 mW cm⁻²) for 2 min. A two-color fluorescence approach was implemented using calcein AM for live cell staining and BOBO-3 iodide for dead cell detection. Briefly, 1 mL of calcein AM (1 μM) was added to 1 μL of BOBO-3 iodide and mixed to prepare a stock solution. The stock solution was diluted by adding 1 mL of live cell imaging solution to prepare the staining solution. Subsequently, 500 μL of staining solution was added to each cell-laden scaffold, and samples were incubated for 30 min at room temperature. Following the incubation, samples were imaged using the DMi8 THUNDER Imager 3D Cell Culture microscope. The live cell channel was set to excitation/emission wavelengths of 470 nm/510 nm, and the dead cell channel was set to excitation/emission wavelengths of 550 nm/610 nm. The live cell heatmaps in GHS samples were generated by converting the images to 8-bit format and adjusting their size to 50 pixels×50 pixels using FIJI ImageJ software (version 1.54f, NIH, MD, USA). To stain cells with Alexa Fluor™ 488 phalloidin and DAPI, samples were first fixed in a PFA solution (4% v/v in ultrapure water) for 45 min. Scaffolds were then incubated in DPBS for 15 min at room temperature, three times, followed by DPBS removal. Next, the samples were permeabilized by incubating in a Triton X-100 solution (0.3% v/v in DPBS) for 10 min, followed by incubation in DPBS for 10 min at room temperature, repeated three times. After DPBS removal, samples were incubated in the Alexa Fluor 488 phalloidin solution (1:400 volume ratio in DPBS) for 60 min at room temperature. Afterward, they were incubated in the live cell imaging solution for 15 min three times. Following this step and for nucleus staining, samples were incubated with a DAPI solution (1:1000 volume ratio in DPBS) for 5 min at room temperature, and then incubated in the live cell imaging solution, for 10 min, two times. Finally, the samples were imaged using the DMi8 THUNDER Imager 3D Cell Culture microscope, equipped with a 25× water contact objective (HC FLUOTAR L 25×/0.95 W VISIR). Nucleus circularity for cells infiltrated into inter- or intraparticle pores in GHS made with porous or nonporous microgels was measured using the maximum intensity projection (MIP) of 3D Z-stacked images. Images were analyzed using FIJI ImageJ software, where they were converted to the 8-bit type, processed using the watershed function, and assessed for nucleus circularity. For this analysis, at least 100 cell nuclei were examined in each scaffold, and three scaffolds were analyzed for each study group.

### Methods—Metabolic Activity Assessment

The PrestoBlue HS cell viability kit was used to assess the cell metabolic activity throughout the cell culture period. Briefly, PrestoBlue was mixed with serum-free DMEM at a 1:10 volume ratio, and 1 mL of the final solution was added to each well of a 24-well plate. Each well had a scaffold (diameter=6 mm and height=3 mm) fabricated using a cell-microgel mixture, containing 4×10³ cells per μL of microgels (packed via centrifugation at 2940×g for 15 s), as mentioned in section 1.11. After incubation for 3 h at a 5% CO₂ atmosphere and 37° C., 100 μL of each supernatant was pipetted into a well of a 96-well microplate, and the fluorescence intensity was measured using a microplate reader (Tecan Infinite M Plex, Mannedorf, Switzerland) at the excitation/emission wavelengths of 530 nm/590 nm. The resulting fluorescence intensity was adjusted with respect to the background signal obtained from the virgin PrestoBlue/media solution incubated in a cell-free well in the 24-well plate for 3 h under a 5% CO₂ atmosphere at 37° C.

### Methods—In Vivo Subcutaneous Implantation Mouse Model

Animal experiments were carried out following the protocol (#02132) approved by The Pennsylvania State University Institutional Animal Care and Use Committee (IACUC). For subcutaneous implantation, based on a power analysis (see Statistical Analysis section), 12 C57BL/6 mice (age=10 weeks, 6 male and 6 female, The Jackson Laboratory, CT, USA) were used. Prior to the surgery, mice were acclimated for 8 days. For the surgery, mice were anesthetized via inhalation of 2% isoflurane carried in oxygen at 5 L min⁻¹. Meloxicam (0.5 mL kg⁻¹) was administered subcutaneously to the anesthetized mice. The dorsal skin was shaved, and surgical site was prepped with ethanol (70% v/v) and Chlorhexidine® scrub (2% v/v). Two pockets were formed on each mouse, and a cuboid scaffold (length=7 mm, width=7 mm, height=3 mm) from each study group was implanted subcutaneously in each pocket. In total, 8 samples for each study group were evenly distributed between male and female mice and randomized to ensure no mice received implants from the same group. Following the surgery, each mouse was placed in an individual cage and housed in a standard day/night light cycle environment, provided with access to food and water. All the mice were checked daily, and their weights were recorded. Mice were euthanized using CO₂ (3 L min⁻¹) 2 weeks after scaffold implantation, and samples along with the surrounding tissues were collected and stored in a 4% v/v PFA solution overnight. Samples were then transferred to an ethanol solution (70% v/v in DPBS).

### Methods—Hematoxylin and Eosin (H&E) and Immunofluorescence Staining

Fixed samples were transferred into cassettes and processed using a Leica TP1020 Automatic Benchtop tissue processor (Leica Biosystems, Germany) with ethanol gradient and xylene immersions, followed by paraffin embedding. To this end, samples were first immersed in 70% v/v ethanol for 30 min, followed by immersion in 85% v/v ethanol for 45 min, and then in 95% v/v ethanol for 40 min (twice). Next, the samples were immersed in 100% v/v ethanol for 40 min (twice). Afterward, the samples were immersed in xylene three times, each for 40 min, and finally immersed in paraffin twice, each time for 45 min. Paraffin-embedded samples were then sectioned using a Shandon™ Finesse™ paraffin microtome (Thermo Fisher Scientific, MA, USA) (thickness ˜7-10 μm) and affixed onto Diamond® white glass microscope slides.

For H&E staining, sections were loaded onto a Leica Autostainer ST5010 XL (Germany) and deparaffinized by heating in an oven at 58° C. for 18 min, followed by three times immersion in xylene, each lasting for 150 s. The sections were then immersed in a descending ethanol gradient, first in 100% v/v and then in 95% v/v, each for 90 s, followed by immersion in tap water for 1 min. Samples were then immersed in staining reagents, including Hematoxylin 560™, Alcoholic Eosin Y 515™, Blue Buffer 8™, and Define®. This was followed by immersion in an ascending ethanol gradient, first in 95% v/v and then in 100% v/v, each for 90 s, and finally in xylene for 4 min.

For immunofluorescence staining analyses, the sections were loaded onto a Leica Autostainer ST5010 XL (Germany). The samples underwent deparaffinization by heating in an oven at 58° C. for 18 min, followed by three immersions in xylene, each lasting 10 min. The sections were then immersed in a descending ethanol gradient. This involved two immersions in 100% v/v ethanol for 3 min each, followed by immersions in 95% and 85% v/v ethanol for 2 min each, and a final immersion in 70% v/v ethanol for 3 min. Dewaxed sections were then washed by two times incubation in tap water for 4 min. Samples were then immersed in PBS for 5 min. Subsequently, the dewaxed samples were subjected to heat-mediated antigen retrieval overnight in a 10 mM citrate buffer (pH=6.0) at 60° C. The sections were blocked with BSA (1% w/v) and TWEEN® 20 (0.3% v/v) in PBS for 1 h at room temperature, which were then stained against α-SMA, CD31, and CD68. To this end, the sections were incubated with recombinant Alexa Fluor© 488 anti-CD31 antibody (1:100), anti-α-SMA (1:100), and Alexa Fluor© 647 anti-CD68 antibody (1:100), each prepared in a 1% w/v BSA solution in PBS, overnight at 4° C. After rinsing with PBS, samples were mounted, counterstained using Fluoroshield™ with DAPI, and imaged using the DMi8 THUNDER Imager 3D Cell Culture microscope. Image analysis involved converting images to an 8-bit type and thresholding using FIJI ImageJ software. Multiple rectangular ROIs (width=400 μm and length=800 μm) were selected for the image analysis. Cell density (%) was measured as the fraction of DAPI fluorescence area over the total area of ROI. Cell infiltration at varying depths was quantified by measuring cell density in 4 adjacent rectangular ROIs (width=200 μm and length=400 μm), starting from the tissue-scaffold interface. This value was then normalized with the summation of cell density across all 4 ROIs. The CD31-, CD68-, and α-SMA-stained area were also measured as the fraction of fluorescence area over the total area of each ROI (width=400 μm and length=800 μm). All the ROIs were selected from the tissue-scaffold interface. The in vivo cell nucleus heatmaps in subcutaneously implanted GHS were generated by converting images to 8-bit type and adjusting their size to 50 pixels×50 pixels using FIJI ImageJ software.

### Methods—Statistical Analysis

G*Power software (version 3.1.9.6) was used to calculate the number of mice (n=8) based on a minimum 80% power, type I error of 0.05, and the effect size of 1.4. Some scaffolds were unable to be analyzed, leading to sample sizes of n=7 for the GHS comprising nonporous microgels or large-pore microgels, and n=6 for the GHS made up of small-pore microgels. The effect size for the n=6 condition was 1.6 (GHS-S) and 1.5 for the n=7 conditions (GHS-L and GHS-N) to maintain 80% power. Sections from all explanted scaffolds were analyzed for cell infiltration and immunofluorescence staining. Statistical significance was determined using unpaired two-tailed t-test or one-way/two-way analysis of variance (ANOVA), followed by Tukey's post-hoc multiple comparison test between the study groups. All the data reported in this study were acquired with at least three iterations (n≥3). The statistical analysis was performed using GraphPad Prism for Windows (version 10.4.0, MA, USA). The level of significance was noted with ns: non-significant p≥0.05, *p<0.05, **p<0.01, ***p<0.001, and ****p<0.0001.

### Results and Discussion

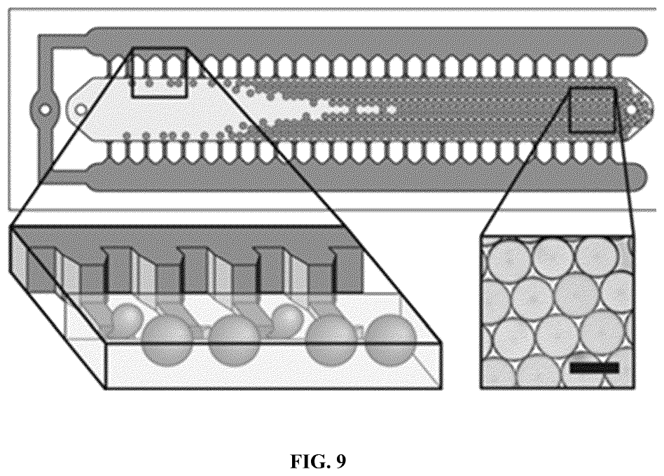

To fabricate porous microgels as the building block of GHS, a homogenous precursor solution, consisting of GelMA, PEG, and LAP is injected into a high-throughput step emulsification microfluidic device. FIG. 9 shows the formation of near-monodispersed droplets suspended in a continuous oil phase, wherein droplets are stabilized using a surfactant. GelMA, a photocrosslinkable protein-based biopolymer, has widely been used to fabricate biocompatible and biodegradable hydrogel biomaterials, containing bioactive peptide sequences that enable cell attachment and matrix metalloproteinase (MMP)-mediated degradation. Similarly, PEG has been selected as a key synthetic polymer for biomedical applications given its tunable physicochemical properties and inertness.

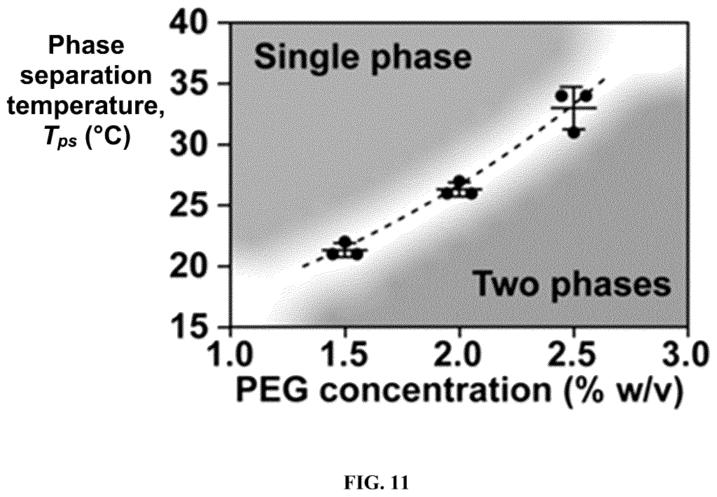

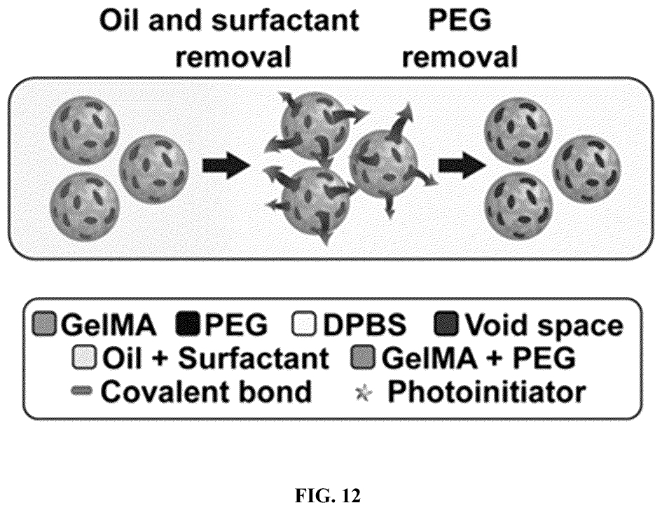

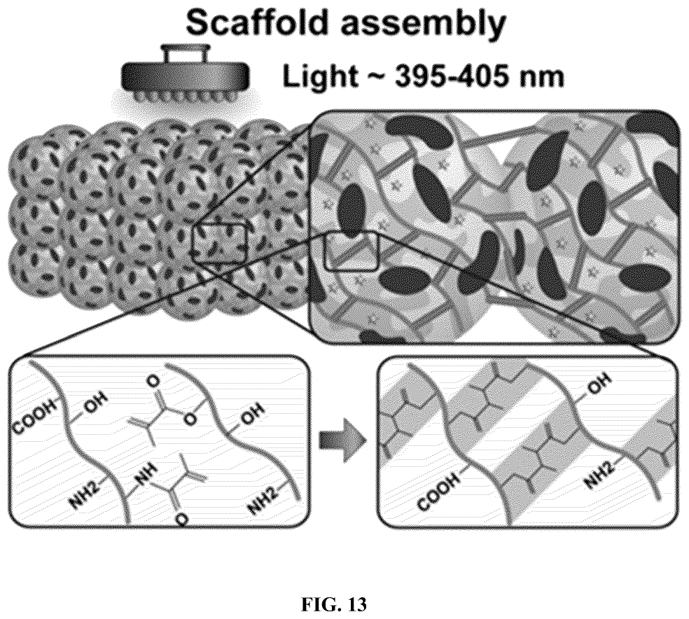

The homogeneous solution of GelMA and PEG shows partitioning behavior, which may be controlled by adjusting the composition or temperature of mixture. Here, the homogenous single-phase droplets containing GelMA-PEG polymers at Tᵢ (initial temperature) are thermally phase separated by lowering the temperature to T_{f} (final temperature). This process is attributed to the increase in the strength of intermolecular hydrogen bonding between gelatin chains upon the addition of PE. As a result, random-coil gelatin chains undergo a partial reversion to triple-helical structures, inherited from collagen. This alteration subsequently reduces the miscibility of gelatin with PEG, as illustrated in FIG. 10. FIG. 11 shows the effect of PEG concentration on the phase separation temperature (T_{PS}) of binary GelMA (7% w/v)-PEG mixture. By cooling homogenous droplets to a temperature below the phase transition temperature, the homogeneous GelMA-PEG solution in droplets phase separates into two distinct phases, which may be halted by GelMA physical gel formation. The phase-separated microgels are then stored at 4° C., and the oil is then removed from the microgel suspension after which PEG polymer chains diffuse out of the microgels, yielding micron-sized pores within individual microgels, as shown in FIG. 12. FIG. 13 presents the formation of GHS, featuring hierarchical interconnected pores at both inter- and intraparticle scales via porous microgel jamming and subsequent chemical crosslinking through light exposure. The formation of covalent bonds via the free radical polymerization of GelMA's methacryloyl groups accounts for both intra- and interparticle crosslinking, yielding stable constructs, as reported for the GHS made up of nonporous microgels.

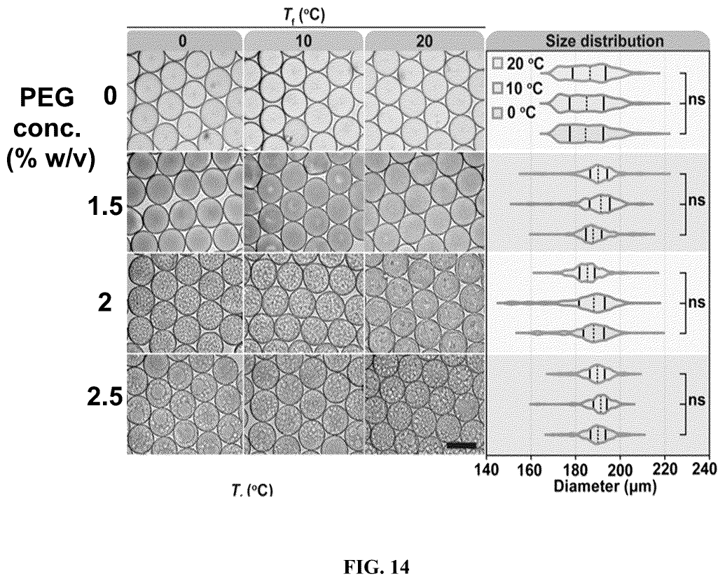

The individual porous microgels are first characterized, followed by GHS formation and characterization. FIGS. 14-17 present the size and pore characterizations of individual microgels after undergoing thermally-induced phase separation. The precursor polymer solution, prepared via mixing PEG (initial concentration=0, 3, 4, or 5% w/v) and GelMA (initial concentration=14% w/v) solutions at a 1:1 volume ratio, is introduced into a high-throughput step emulsification microfluidic device as a dispersed phase, resulting in the formation of droplets in a continuous oil phase. Droplets are then collected in a water bath at 37° C. (Tᵢ) to remain as a homogenous GelMA-PEG mixture. To induce phase separation for fabricating porous microgels, droplets are immersed in an aqueous bath at a fixed T_{f} (0, 10, or 20° C.), which is monitored using a thermometer. The applied temperature gradient reduces temperature at varying cooling rates. As the GelMA-PEG mixture is cooled below T_{PS}, the thermodynamic incompatibility between GelMA and PEG leads to liquid-liquid phase separation.

As the size of droplets and subsequent microgels produced by the step emulsification device is controlled by the channel height (˜60 μm), we anticipate a consistent and narrow size distribution for the phase-separated microgels despite varying final (water bath) temperatures. FIG. 14 shows the bright-field microscopy images of phase-separated microgels at varying T_{f} and PEG concentrations, along with their corresponding size distribution. A narrow size distribution with an average diameter of 187±10 μm across all PEG concentrations (0, 1.5, 2, 2.5% w/v) and T_{f} (0, 10, or 20° C.) (n>5000 over all conditions) is obtained. Also, the particle size analysis at each PEG concentration (n>300) shows no significant difference among microgel groups prepared at varying T_{f}.

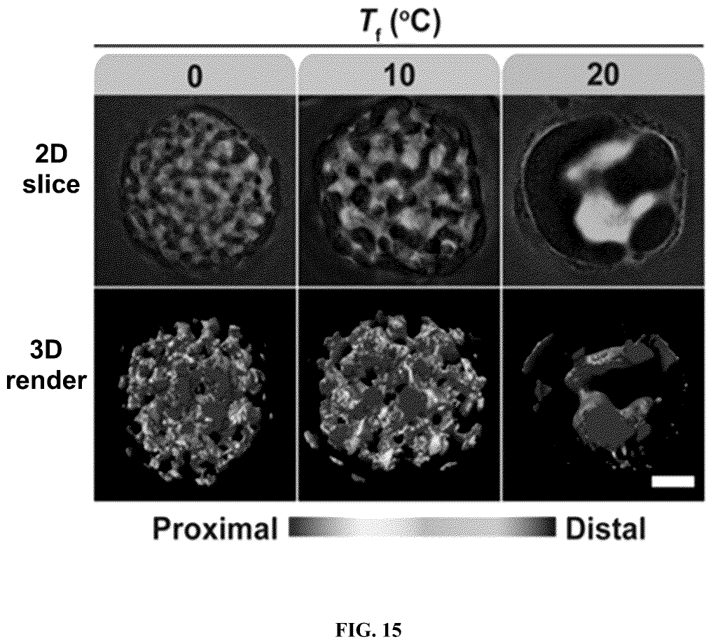

To characterize the porous structure of physically and chemically crosslinked individual microgels and to investigate the effect of cooling rate on phase separation, a high-molecular weight fluorescent dextran (average molecular weight=2 MDa) is used, occupying the intraparticle void spaces. FIG. 15 presents 2D slices near the center of fluorescently labeled microgels (initially contained 2% w/v PEG), as well as their 3D renderings wherein the 3D-constructed images show interconnected pores inside individual microgels. Importantly, the cooling rate of microgel suspension during the immersion step influences the phase separation process and, consequently, the resulting microgel pore structure. The data are averaged and fitted using a single-phase decay formula (n=3): T(t)=(Tᵢ−T_{∞})e^{-λt}+T_{∞}, where T(t) is the microgel suspension temperature at any given time, Tᵢ is the initial microgel suspension temperature, λ is the decay constant, t is the time elapsed, and T_{∞} is the T value at infinite time, which is close to T_{f}. At T_{f}=0, 10, or 20° C., the non-linear fit to the data results in λ₀=0.07434 s⁻¹ with R²=0.997, λ₁₀=0.04425 s⁻¹ with R²=0.979, or λ₂₀=0.03808 s⁻¹ with R²=0.995, respectively. The subscript of indicates the T_{f} for each condition. As T_{f} decreases from 20° C. to 0° C., the cooling rate or decay constant increases significantly, which causes the GelMA-PEG mixture to pass through T_{PS} more quickly and reach the GelMA physical gel formation temperature faster, thereby halting phase separation and yielding unique phase separated patterns. Previous studies have demonstrated that in the absence of gel formation, phase separation patterns evolve over time as each phase self-organizes to minimize the total interfacial energy. When the components in the aqueous phase undergo both phase separation and rapid gel formation (e.g., GelMA), they lack the time required to fully reach an energy-minimized configuration, leading to incomplete phase separation. Consequently, the lowest decay constant produces patterns closer to the final uninterrupted phase-separated state, while the highest decay constant results in patterns resembling the initial states of phase separation.

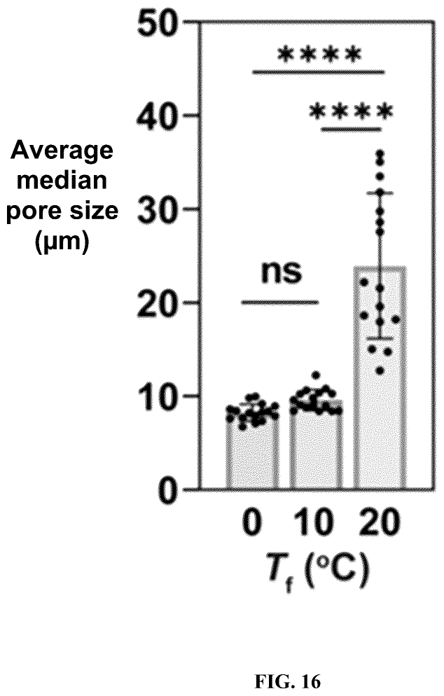

The porous architecture of individual microgels was further analyzed using a MATLAB code. FIG. 16 shows the average median pore size of porous microgels. Microgels that undergo phase separation at the lowest decay constant (i.e., λ₂₀=0.03808 s⁻¹) have a considerably higher average median pore size (˜24±8 μm) than their counterparts subjected to phase separation at 0° C. or 10° C., which had an average median pore size of ˜8±1 or ˜9±1 m, respectively. Moreover, there is no significant difference in the average median pore size of the individual microgels phase separated at 0° C. and 10° C. When microgels are subjected to a higher cooling rate, they reach the gel formation temperature more rapidly, impeding complete phase separation, which leads to the formation of smaller pores.

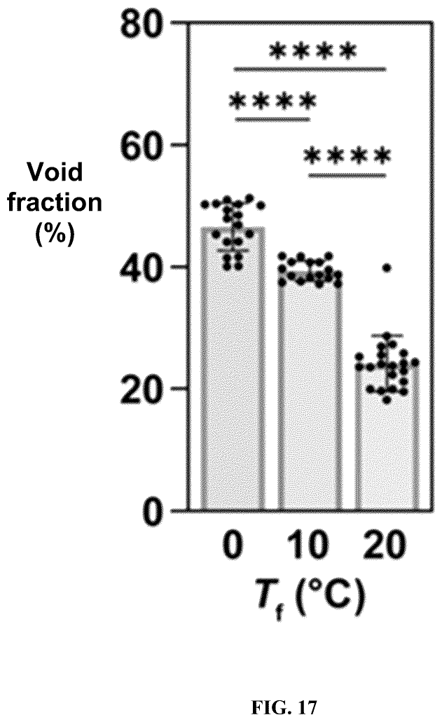

FIG. 17 presents the void fraction of porous microgels. The void fraction, defined as the ratio of fluorescently labeled void space to total microgel volume, is significantly affected by the cooling rate. The void fraction of microgels prepared at the cooling rate with the highest decay constant (i.e., λ₀=0.07434 s⁻¹) is ˜46±4%. As the decay constant decreases by increasing T_{f}, the void fraction monotonically decreases from ˜39±2% (λ₁₀=0.04425 s⁻¹) to ˜24±5% at the lowest decay constant (λ₂₀=0.03808 s⁻¹). By reducing the final temperature, the heat transfer rate between the microgel suspension and surrounding environment increases. Consequently, when microgels are subjected to a lower cooling rate (e.g., T_{f}=20° C.), the slower heat transfer rate allows for more complete phase separation until the physical gel formation temperature is reached. The difference in the void fraction of microgels prepared at the lowest cooling rate (λ₂₀=0.03808 s⁻¹) and those prepared at the highest cooling rate (i.e., λ₀=0.07434 s⁻¹) is attributed to gel formation during phase separation. This process can lead to the formation of PEG-trapped states, which affect the interconnection of pores within the microgels, potentially leaving PEG residues inside and resulting in a lower void fraction in microgels with the lowest decay constant.

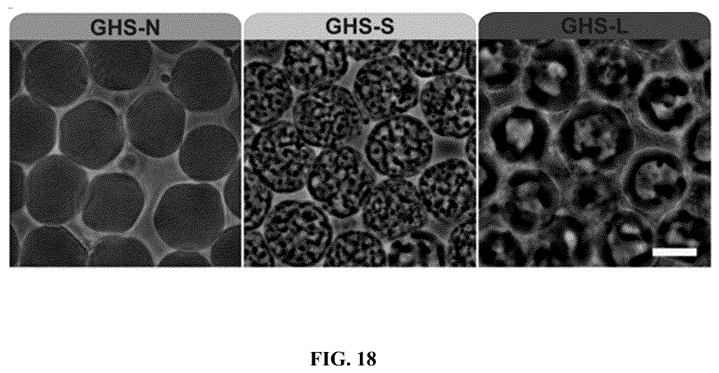

FIGS. 18-25 shows the pore and mechanical characteristics of GHS, fabricated from nonporous or porous microgels (initially contained 2% w/v PEG) that are phase-separated at 0° C. or 20° C. FIG. 18 presents intra- and inter-particle pores within the GHS made up of nonporous microgels (GHS-N), small-pore microgels (GHS-S), and large-pore microgels (GHS-L), visualized via a high-molecular weight fluorescent probe. While the fluorescent probe successfully penetrates the porous microgels, enabling the visualization of additional void spaces in GHS made up of porous microgels, nonporous microgels do not allow fluorescent molecule penetration. In conjunction with fluorescence microscopy, the structure of GHS is imaged using SEM. The results show that the non-porous microgels do not have any pores, and compared with the large-pore microgels, the small-pore counterparts have more pores on their surfaces. However, the pores on the surface of large-pore microgels are larger than those on the small-pore counterparts. These findings highlight the structural differences between the small- and large-pore microgels compared with the nonporous counterparts.

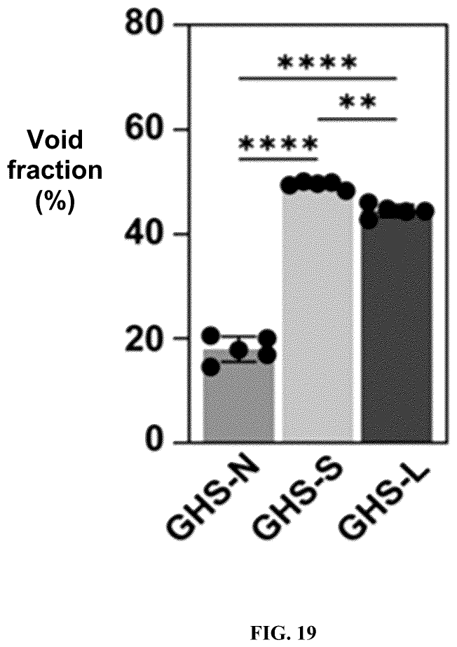

FIG. 19 shows the void fraction of GHS comprising nonporous or porous microgels. The micron-sized pores of scaffolds are quantified by analyzing the fluorescence images using a MATLAB code. The results show a significant difference between the void fraction of GHS-N, GHS-S, and GHS-L, wherein the GHS-S has the highest void fraction (49±1%), followed by the GHS-L (44±1%), and GHS-N(18±2%). The higher void fraction in GHS-S and GHS-L is attributed to the additional void spaces formed through the phase separation of GelMA-PEG mixture inside individual microgels, followed by PEG polymer removal. The theoretical porosity in scaffolds, φ_{theoretical}, can be calculated using the following formula: φ_{theoretical} (%)=φ_{GHS} (%)+φ_{μgel} (%) [100%−φ_{GHS} (%)], where φ_{GHS} is the average porosity (void fraction) of non-porous GHS (˜18%), and φ_{μgel} is the average porosity of individual microgels. The microgel porosity is multiplied by (100%−φ_{GHS}) to account for the area expected to be occupied by microgels. When comparing the φ_{theoretical} with φ_{Observed} for each study group, a difference of 6-7% is observed, which is not surprising, given that φ_{Observed} is measured experimentally. In contrast, when comparing φ_{theoretical} between GHS-S and GHS-L, an 18% difference is expected; however, the observed difference is ˜5%. This difference is attributed to underestimated porosity in GHS-S. At low-magnification images, some of the thinner pores in GHS-S do not produce a strong enough signal to be distinguished from random noise, causing them to remain unlabeled. As a result, the observed porosity is lower than the theoretical porosity calculated from the individual microgel porosity.

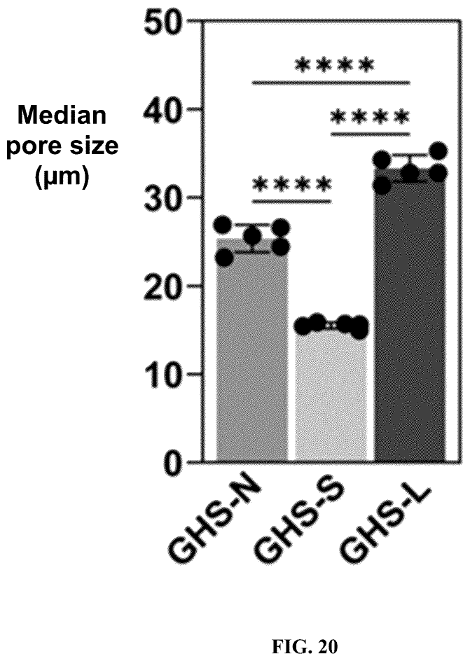

FIG. 20 presents the effect of microgel pore size on the average median pore size of GHS, where maximum microgel-microgel contact is observed. The average median pore size is 25±2 μm for GHS-N, 16±1 μm for GHS-S, and 33±2 μm for GHS-L. In comparison with the pore structure of GHS-N, GHS-S has a higher number of intraparticle pores with smaller sizes, yielding a lower average median pore size. Conversely, GHS-L has a higher number of large intraparticle pores than GHS-N, resulting in a higher average median pore size. Overall, these outcomes show that the hierarchical porous structure of GHS may be tailored via varying pore features of individual microgels, which is not trivial by using nonporous spherical microgels.

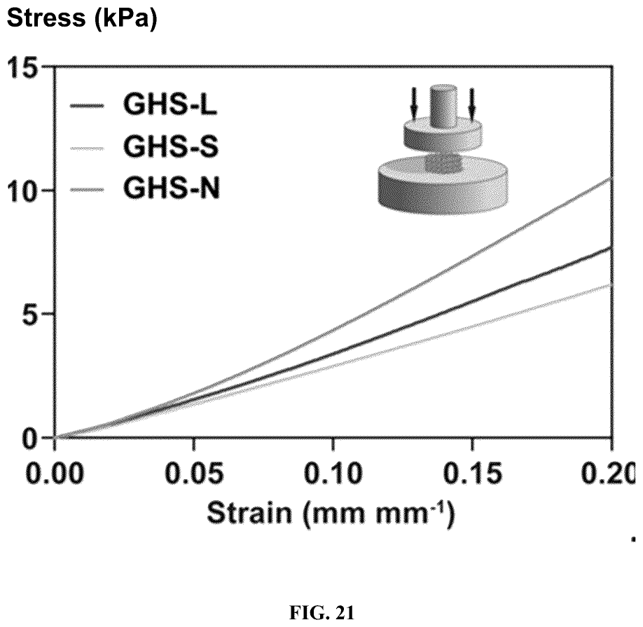

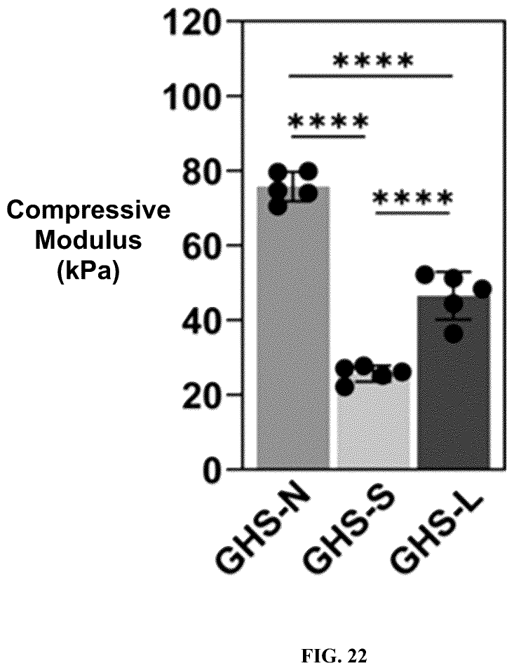

The mechanical and rheological properties of GHS, fabricated with nonporous or porous microgels, are evaluated using compression and oscillatory rheology tests, as presented in FIGS. 21-24. FIG. 21 shows the stress-strain curves, acquired via applying a compressive load to the scaffolds. In GHS-N and at any given strain, the stress is higher than that in GHS-S and GHS-L. FIG. 22 presents the compressive modulus of GHS, measured from the slope of compressive stress-strain curves in the linear elastic region, located at ˜5-15 mm mm⁻¹ compressive strain. The compressive modulus of scaffolds is inversely correlated with the void fraction: the scaffold with the highest void fraction, i.e., GHS-S, has the lowest modulus, and GHS-N has a significantly higher modulus than GHS-S and GHS-L.

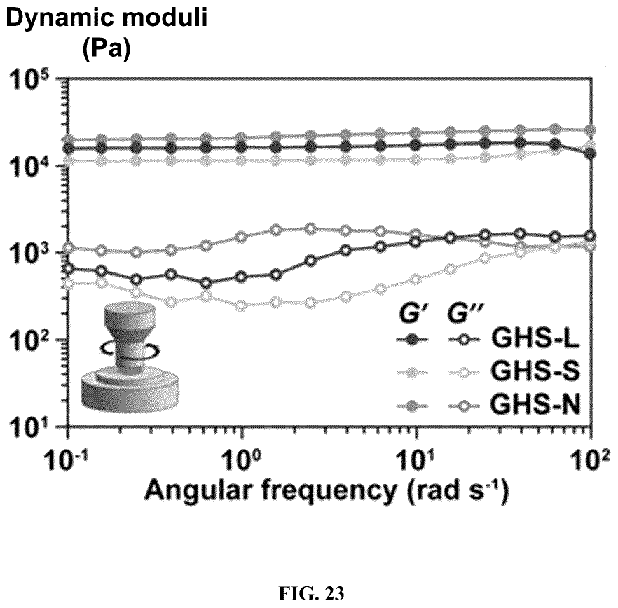

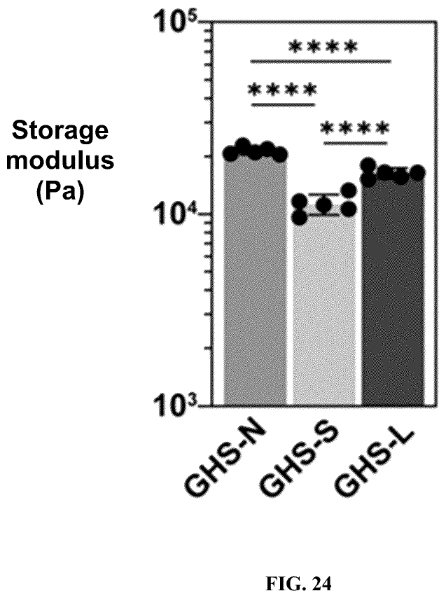

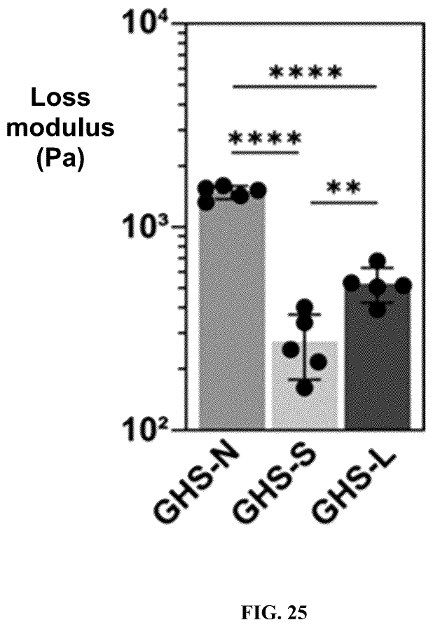

FIG. 23 shows the viscoelastic characteristics of scaffolds, evaluated via oscillatory rheology tests. The LVR is identified via an oscillatory strain sweep test at a constant frequency (1 rad s⁻¹). Then, storage (G′) and loss (G″) moduli are measured at a constant oscillatory strain of 0.1%, which is in the LVR, from 0.1 to 100 rad s⁻¹. Similar to the compression behavior reported earlier, the G′ of GHS-N is higher than that of GHS-S and GHS-L. FIG. 24 presents the average G′ of different study groups, measured at 0.1% strain and frequency of 1 rad s⁻¹. The average G′ of GHS-N is significantly higher than that of GHS-S or GHS-L, and GHS-L has a significantly higher G′ than GHS-S. FIG. 25, shows the average G″ of scaffolds at 0.1% oscillatory strain and frequency of 1 rad s⁻¹. A consistent trend akin to the G′ versus angular frequency is observed across all study groups for the G″. Additionally, in all study groups, G″ is at least an order of magnitude lower than the corresponding G′, which is a characteristic of gels.

The descending trend of compressive and storage moduli observed in the GHS, fabricated using the porous microgels compared with the counterpart made up of nonporous microgels may be attributed to the additional void spaces introduced by the porous microgels. Previous studies have shown that the GHS, fabricated with microgels of varying sizes, have significantly lower storage and compressive moduli compared with nonporous scaffolds. This difference has been attributed to the larger (macroscale) void spaces in GHS, which is absent in bulk hydrogel scaffolds. The larger void space in GHS results in lower interparticle contact area and consequently weaker interparticle contact among the microgels. A similar phenomenon may occur within individual microgels. In porous microgels, unlike their nonporous counterparts, the void spaces lead to a reduction in the mechanical strength. Such weakened mechanical strength at the microgel level affects the mechanical properties of the entire GHS.

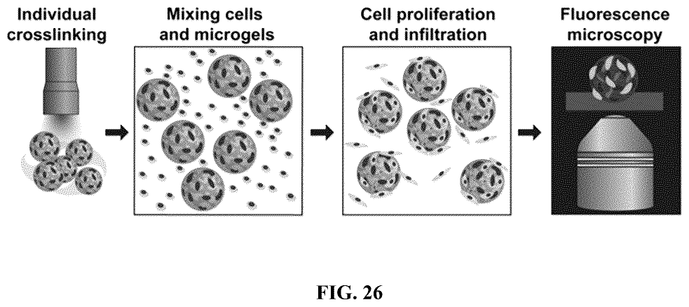

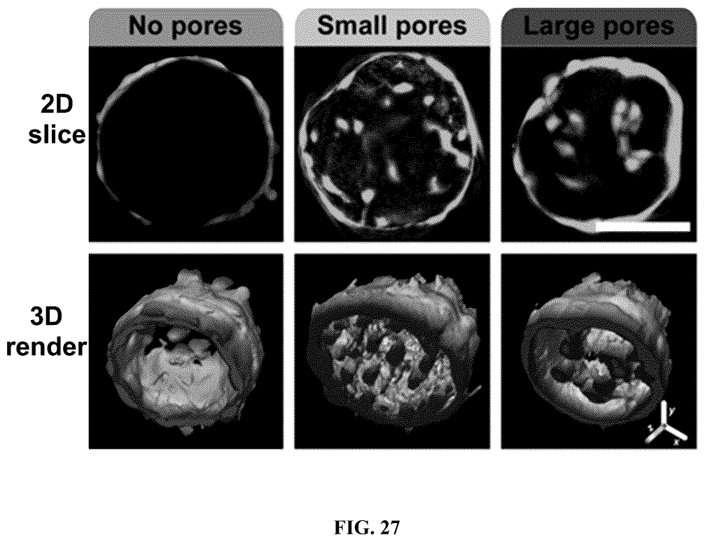

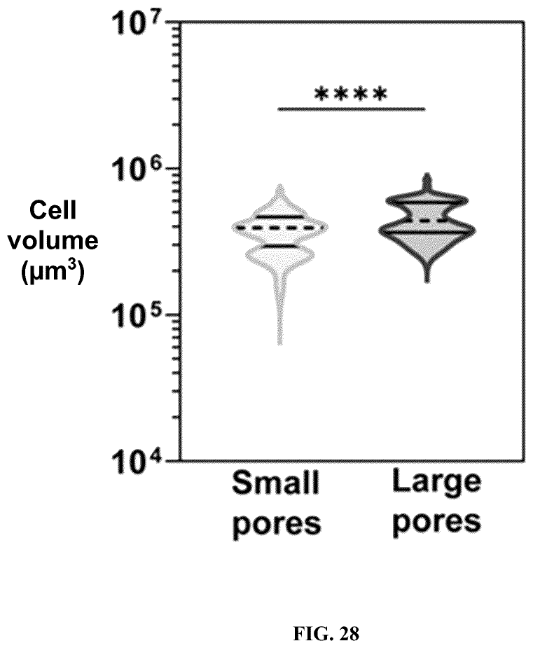

To evaluate the effect of microgel porous structure on the viability, proliferation, and migration of cells, in vitro studies are conducted using individual microgels, as well as the GHS, assembled from porous or nonporous microgels. FIG. 26 presents the schematic illustration of NIH/3T3 murine fibroblast cell interaction with individually photocrosslinked microgels. Phase-separated droplets are first physically crosslinked, followed by individual photocrosslinking. NIH/3T3 murine fibroblast cells are mixed with the microgels and cultured for 2 days. Then, cells are labeled with the calcein AM cell-permanent dye, and fluorescence images are acquired to study cell-microgel interactions, including adhesion and migration around/within microgels. FIG. 27 shows 2D cross-sectional and 3D-constructed images of microgels. Cells only adhere to the outer surface of nonporous microgels; however, they not only adhere to the porous microgel exterior, but also migrate into the intraparticle void spaces through the open pores. FIG. 28 presents the total volume of infiltrated cells into individual porous microgels. Cell volume, defined as the volume occupied by cells within the porous microgels, is calculated for small- and large-pore microgels. Accordingly, the cell volume in the large-pore microgels is significantly higher than that in the small-pore counterpart. These results are consistent with microgel pore characterization provided earlier: the median pore size of microgels that undergo phase separation with the lowest temperature decay constant (λ₂₀=0.03808 s⁻¹) are significantly higher than those prepared at the highest decay constant (λ₀=0.07434 s⁻¹), facilitating cell migration in the large-pore microgels compared with the small-pore counterparts.

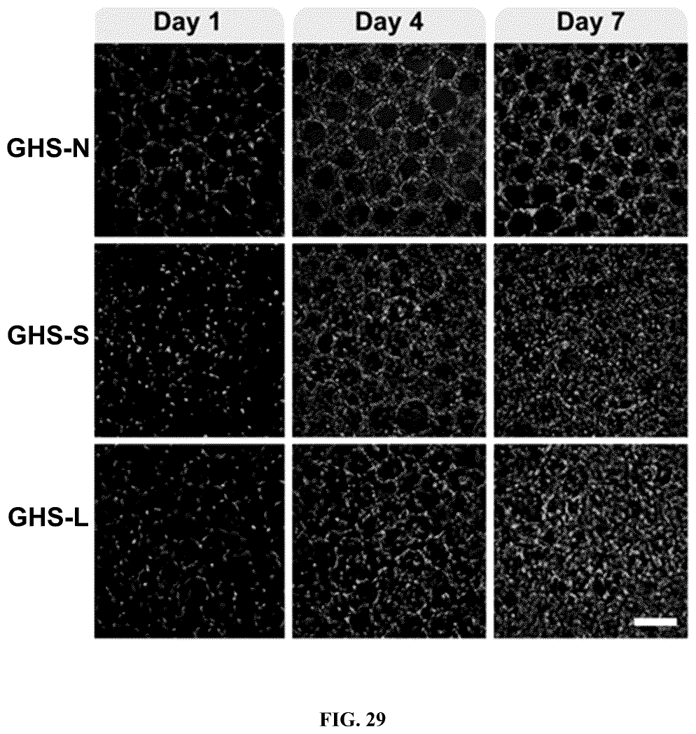

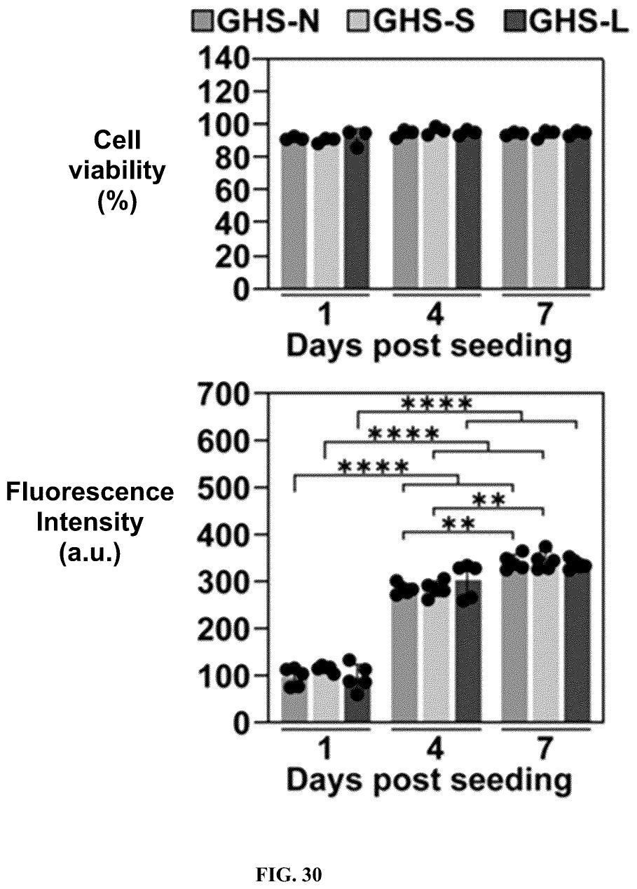

Cell viability, defined as the ratio of area occupied by live cells to the total area of live and dead cells, is characterized in GHS-N, GHS-S, and GHS-L. FIG. 29 shows the live/dead images of cell-laden scaffolds, fabricated using cell-microgel suspensions. Over the culture period, the number of live cells increases, as shown by the growing area of live cells adhering to the microgels and proliferating. Additionally, a low number of dead cells are observed in the scaffolds. Furthermore, in the GHS made up of porous microgels, a more uniform cell distribution is observed compared with the GHS made with nonporous microgels. This shows successful cell infiltration into the void spaces of porous microgels, as expected based on the successful cell infiltration into individual microgels. FIG. 30 shows the high viability of cells (>95%) in GHS made up of porous or nonporous GelMA microgels with no significant difference among the study groups, indicating the biocompatibility of porous GHS in vitro.

To further investigate the in vitro cell behavior in the GHS, the metabolic activity of cells is assessed using the PrestoBlue assay during cell culture. FIG. 30 presents the metabolic activity of cells on days 1, 4, and 7 after forming GHS using cell-microgel suspensions. On day 7, the assay shows the cell metabolic activity is at least tripled across all groups. While the overall growth in metabolic activity is expected because of the interparticle void spaces in GHS, increasing the transport of oxygen, nutrients, and cellular waste, the incorporation of porous microgels in GHS does not significantly alter the cell metabolic activity compared with the nonporous microgels. A reason for this observation may be that GHS enable sufficient metabolite diffusion despite the differences in void fraction and pore size among the study groups.

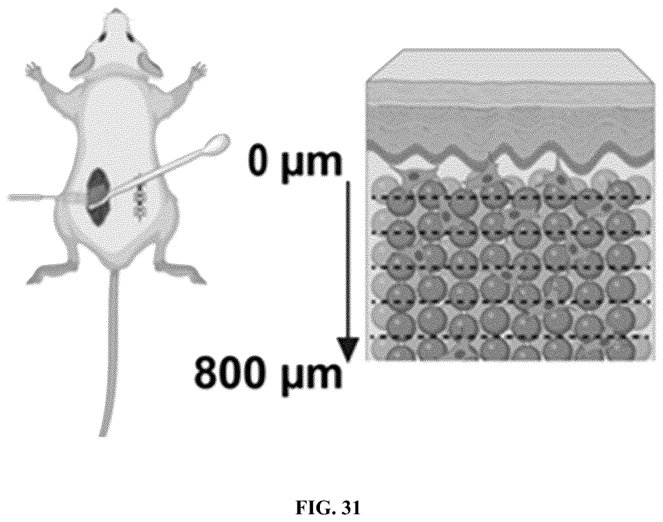

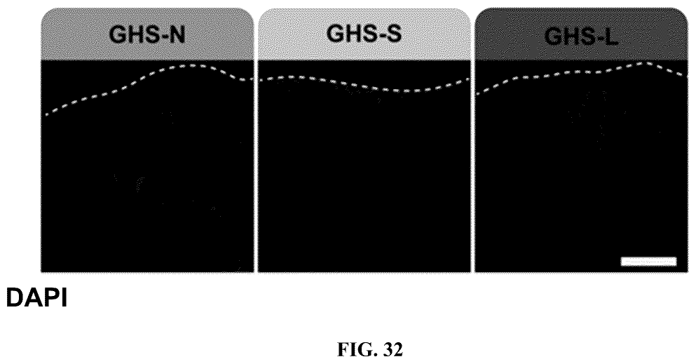

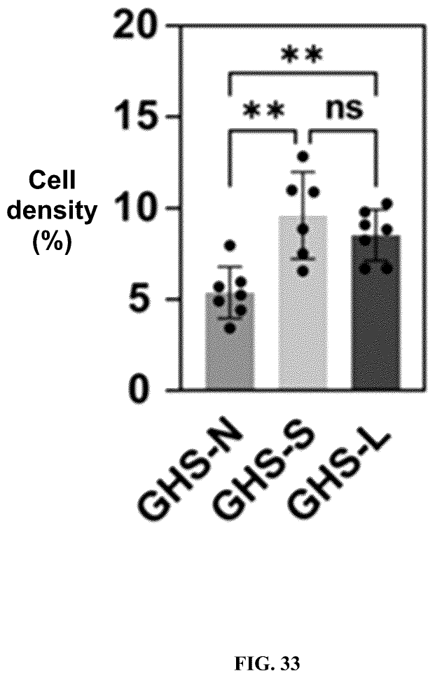

Cell infiltration is a critical stage in facilitating the integration of scaffolds with a host tissue and promoting tissue regeneration. We hypothesize that surpassing the void fraction limit of GHS-N using porous microgels may provide additional space and surface area, enhancing the recruitment of endogenous cells and thus facilitating the scaffold integration within the host tissue. To assess cell infiltration in GHS made up of porous or nonporous microgels, we subcutaneously implant the scaffolds into pockets, formed on the dorsal skin of mice, as schematically shown in FIG. 31. Following a two-week period, all scaffolds are explanted, and subsequent immunofluorescence and H&E staining analyses are conducted. FIG. 32 shows the extent of cell infiltration into scaffolds, as indicated by DAPI-stained cell nuclei in 7-10 μm-thick sections. Compared with GHS-N, GHS-S and GHS-L have higher cell density. The uniformity in cell distribution observed in GHS composed of porous microgels may be attributed to the pores at both inter- and intra-microgel levels, which is consistent with the in vitro results. The hierarchical pores enable the cells to occupy not only the void spaces among the microgels but also within the porous microgels. For the quantitative analysis of cell infiltration in the scaffolds, cell density is measured across different ROIs (length ˜800 μm and height ˜400 μm), starting from the tissue-scaffold interface, marked with a dashed line in FIG. 32. FIG. 33 presents cell density in the explanted samples, where the fraction of DAPI fluorescence area over the total area of ROI in GHS-S(9.6±2.4%) and GHS-L (8.5±1.4%) is significantly higher than that in GHS-N(5.4±1.4%). This shows an approximately 78% and 57% increase in cell infiltration in GHS-S and GHS-L, respectively, compared with GHS-N. We attribute the observed trend in cell density across the study groups to the differences in void fraction. Specifically, GHS-S and GHS-L have a ˜170% and ˜140% increase in void fraction compared with GHS-N, respectively. Such a significant increase in void fraction, provides more surface area for cell adhesion, thereby promoting higher cell infiltration into the scaffolds.

FIG. 34, show the quantitative analysis of cell infiltration in varying depths of scaffolds, measured using ROIs with length ˜400 μm and height ˜200 μm. Cell infiltration in all study groups within the first layer of scaffolds (˜200 μm) from the interface is significantly higher than the other layers. No statistically significant difference is observed between the remaining layers in each group. In the top layer, cell infiltration in GHS-N, GHS-S, and GHS-L is 46±13%, 61±19%, and 66±17% of the total cell area in each study group, respectively. The higher cell density observed in the first layer of GHS-S and GHS-L compared with the other layers within the same sample may be attributed to the higher void fractions in the porous microgels. This creates more void spaces in the first layer for cells to infiltrate before moving deeper into the scaffold. Although a gradual decrease in the cell infiltration is noted within the scaffolds (depth=200-800 μm) across all samples, no significant difference is observed across the remaining layers.

To compare cell infiltration at each depth among the study groups, the cell nucleus area at each interval (depth=200 μm) of GHS-S and GHS-L is normalized with the average cell area in the corresponding layer in GHS-N. It is noteworthy that the first two layers in GHS-S undergo ˜240% and ˜170% increases in cell infiltration compared with the same layers in GHS-N, respectively. Similarly, in GHS-L, the first two layers undergo a ˜230% and ˜135% increase in cell infiltration, respectively, compared with the corresponding layers in GHS-N. This increase is attributed to the additional void spaces within individual microgels, which facilitate endogenous cell infiltration in GHS. Moreover, to compare cell infiltration in GHS-S and GHS-L, the cell nucleus area in each depth of GHS-L is normalized with the average cell area in the corresponding layer in GHS-S. Although the normalized cell area does not significantly change across varying depths of the scaffolds, at least a ˜3% increase in cell infiltration is observed in the first three layers of GHS-S compared with GHS-L. This increase is possibly because of the larger median pore size of GHS-L, which facilitates cell infiltration into the void spaces of individual microgels.

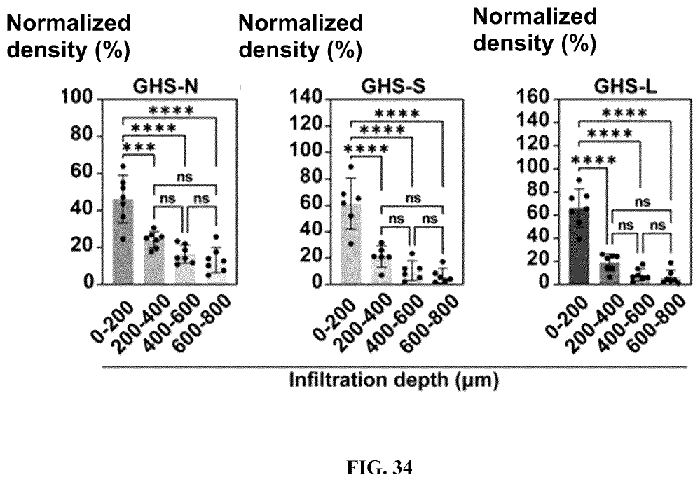

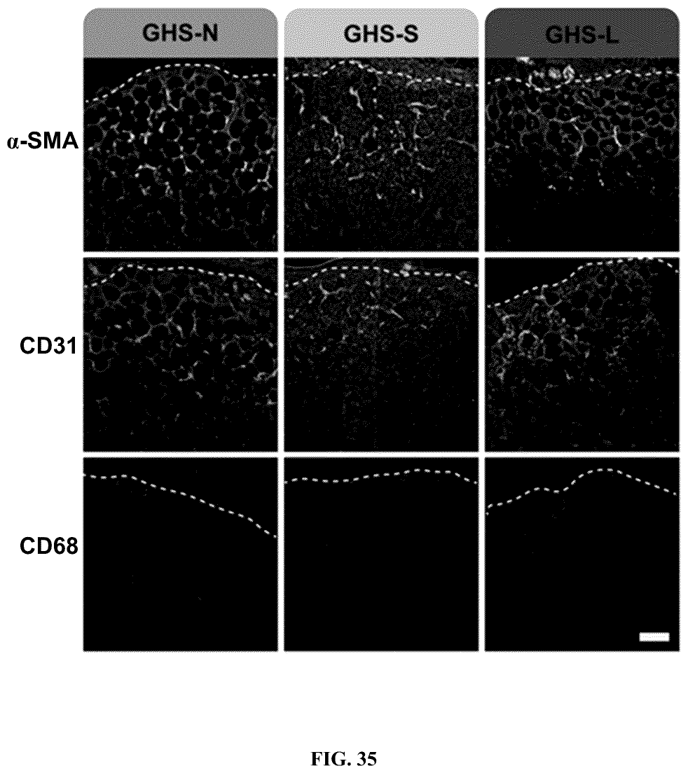

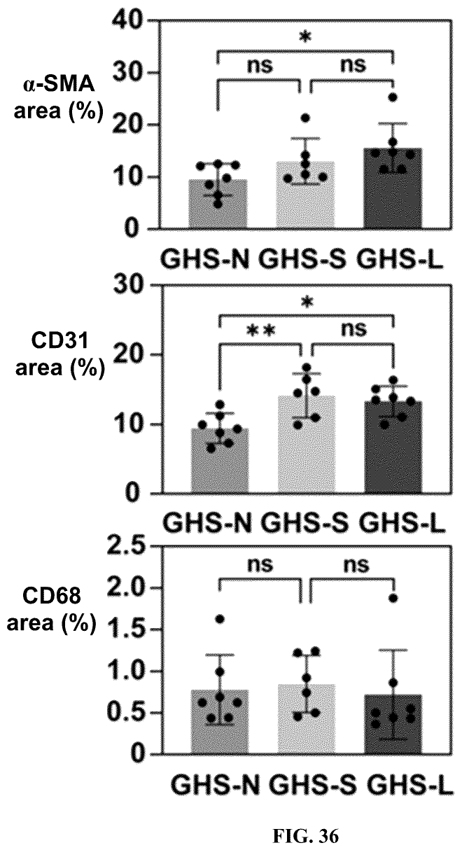

Subsequently, immunofluorescence staining is conducted to identify the type of cells that infiltrated the scaffolds, made up of porous or nonporous microgels. FIG. 35 presents the immunofluorescence images of varying cell populations, including endothelial cells, myofibroblasts, and macrophages, infiltrated into the scaffolds, which are stained by CD31, α-SMA, and CD68 markers, respectively. FIG. 36 presents the average of α-SMA-stained area normalized with the total ROI area. In GHS-L, myofibroblast infiltration is 15.6±4.7%, showing a ˜64% increase compared with GHS-N(9.5±3.0%), while no significant difference is observed between GHS-L and GHS-S(13.0±4.4%). FIG. 36 shows CD31-stained area in the GHS, made up of porous or nonporous microgels. Endothelial cell population is significantly higher in GHS-S(14.1±3.2%) and GHS-L (13.3±2.2%) compared with GHS-N(9.4±2.2%), showing 50% and ˜41% increase in infiltration, respectively. FIG. 36 presents the macrophage infiltrated area in varying study groups. While a higher stained area for CD31 and α-SMA is observed in GHS-S and GHS-L compared with GHS-N, no significant difference is observed for the macrophage infiltration across the study groups. Overall, the results imply that the increased void fraction in the GHS composed of porous microgels leads to higher cell infiltration compared with the GHS made up of nonporous microgels. Furthermore, the increased endothelial and myofibroblast recruitment underscores the potential of these scaffolds for tissue regeneration applications, where vascularization is critical.

### Example 3: Cell Adhesion and Self-Assembly of Cell-Microgel Biohybrid Spheroids

### Materials

Novec™ 7500 Engineered Fluid was purchased from 3M (MN, USA). HUVEC and NIH/3T3 cells were purchased from ATCC (VA, USA). BRAND® microplates (BRANDplates®, inertGrade, low-binding, 96 wells, 330 μL, round bottom, transparent) were purchased from BRAND GMBH+CO KG (Germany). Corning® Matrigel® Matrix was purchased from Corning (NY, USA). HyClone characterized fetal bovine serum (FBS) and HyClone penicillin-streptomycin 100× solution (P/S) were purchased from Cytiva (UT, USA). Polydimethylsiloxane (PDMS, SYLGARD 184 silicone elastomer kit) was purchased from Dow Corning (MI, USA). Biopsy punches (1.5 mm with plunger system) were purchased from Integra Miltex (NY, USA). KMPR 1000 series photoresists were purchased from Kayaku Advanced Materials (MA, USA). EGM™-2 Endothelial Cell Growth Medium-2 BulletKit™ was purchased from Lonza (Switzerland). Ultra-pure Milli-Q water (electrical resistivity ≈18 MΩ cm at 25° C.) provided from Direct-Q 5 UV remote water purification system, Millipore Corporation (MA, USA). Mesenchymal stem cell growth medium 2 was purchased from PromoCell (Germany). Disposable cell scrapers (Biologix, disposable, polyethylene, sterile, handle length: 180 mm, blade length: 18 mm), micro centrifuge tubes (Celltreat, 1.5 mL, clear, polypropylene), centrifuge tube (Celltreat, 15 mL, sterile), and cell strainer (Celltreat, 40 μm, polypropylene, sterile) were purchased from Neta Scientific Inc. (NJ, USA). Deuterium oxide (D₂O, deuteration degree 99.95%), gelatin (type A from porcine skin, gel strength ˜300 g Bloom), Methacrylic anhydride (MAA, contains 2,000 ppm topanol A as inhibitor, 94%), lithium phenyl-2,4,6-trimethylbenzoylphosphite (LAP, >95%), fluorescein isothiocyanate(FITC)-dextran (average molecular weight=2 MDa), trichloro(1H,1H,2H,2H-perfluorooctyl)silane (F-silane, 97%), 4-(4,6-Dimethoxy-1,3,5-triazin-2-yl)-4-methylmorpholinium chloride (DMTMM, ≥97.0%), and Human MSCs (Bone Marrow) were purchased from Sigma. Pico-Surf® (1% in Novec™ 7500) was purchased from Sphere Fluidics (UK). 1H, 1H, 2H, 2H-perfluoro-1-octanol (PFO) and a variety of cell culture and staining reagents including Gibco™ Dulbecco's phosphate buffered saline (DPBS, 1×, no calcium, no magnesium), Dulbecco's modified Eagle medium (DMEM), Trypsin-EDTA (0.25%, with phenol red), Pierce™ 16% formaldehyde (w/v, methanol-free), PrestoBlue™ cell viability reagent, LIVE/DEAD™ cell imaging kit (contains Calcein AM, cell permeant dye as live cell indicator and BOBO-3 Iodide as dead cell indicator), Alexa Fluor™ 647 hydrazide, Hoechst 34580, CellTracker™ Fluorescent Probes (Green CMFDA and Red CMTPX Dyes), and 4′,6-Diamidino-2-Phenylindole (DAPI) were purchased from Thermo Fisher Scientific (MA, USA). Dialysis membrane (12-14 kDa molecular weight cutoff) was purchased from Spectrum Laboratories (NJ, USA). Silicon wafers (100 mm diameter, 500 μm thickness, UniversityWafer, Inc., MA, USA). Sterile disposable filter units with (filter capacity 500 mL, pore size 0.2 μm), disposable pipetting reservoirs, plain microscope slides (thickness 1.0 mm, length×width 75 mm×25 mm), petri dishes (sterile, 60 mm×15 mm, polystyrene), and ethanol (200 proof/100%) were purchased from VWR (PA, USA).

### Methods—Gelatin Methacryloyl (GelMA) Synthesis

Gelatin methacryloyl (GelMA) was synthesized according to our previously established protocols. DPBS (400 mL) was heated to 50° C., and 20 mg of gelatin was dissolved via stirring at 200 rpm. MAA (16 mL) was then added dropwise to the mixture being stirred at 50° C. The reaction was stopped after 3 h by adding 400 mL of DPBS. The solution was then dialyzed against ultra-pure Milli-Q water for 10 days at 40° C. to remove unreacted MAA. A clear solution was obtained, sterile filtered using filtration units, and frozen at −80° C. Finally, the frozen GelMA was freeze-dried using Labconco FreeZone 4.5 L-84 C Benchtop Freeze Dryer (Labconco Corporation, MO, USA) at a collector temperature of ˜82.4° C. under 0.009 mbar of pressure to yield a white solid.

### Methods—Proton Nuclear Magnetic Resonance (1H NMR) Spectroscopy

GelMA synthesis was confirmed by ¹H NMR spectroscopy using a 500 MHz Bruker NEO instrument (MA, USA) at the Pennsylvania State University NMR facility. Gelatin and GelMA samples (40 mg each) were dissolved in 2 mL of D₂O, then heated at 37° C. for 2 h to ensure complete dissolution. Vinyl group peaks, indicative of methacryloyl modification and absent in gelatin, were identified between 5.5 to 6.5 ppm using TopSpin software (version 4.0.7, Bruker, MA, USA).

### Methods—GelMA Fluorescent Conjugation

To conjugate GelMA with a fluorophore, 1 g of GelMA was dissolved in 50 mL of DPBS under continuous stirring at 200 rpm at 40° C. until fully dissolved. Then, 22 mg of DMTMM was introduced into the mixture, followed by the addition of Alexa Fluor 647 hydrazide solution (48 μL). The conjugation reaction was allowed to proceed at 40° C. for 2 h. Then, the solution was dialyzed against Milli-Q water for 3 days at 40° C. to remove unreacted components. After dialysis, the solution was frozen at −80° C., followed by lyophilization to obtain the fluorescently conjugated GelMA in a solid form. The lyophilized product was stored in the dark.

### Methods—Microfluidic Device Fabrication

High-throughput step emulsification microfluidic devices were fabricated at the nanofabrication facilities at the Pennsylvania State University. Two- or three-layer master molds were fabricated on silicon wafers using the KMPR 1000 series as the negative photoresists. The first layer was spin-coated with KMPR 1005, KMPR 1025, or KMPR 1035 for the mold fabrication of small, medium, or large droplets, respectively, according to the manufacturer guidelines. This resulted in layer heights of 8, 27, or 60 μm, respectively. Following this, subsequent layers were deposited using KMPR 1035, designed to be 2-3 times the size of the anticipated droplet, to provide ample space for droplet formation and mobility. The devices were then fabricated using the PDMS by mixing the base and crosslinker at a ratio of 10:1, vacuum degassing, pouring onto the nanofabricated molds, degassing again, and curing at 80° C. for 2 h. The devices were then punched with a 1.5 mm biopsy punch for the inlets and outlets, bonded to a glass microscope slide after air plasma treatment at 400 mTorr for 45 s, followed by an ε-silane (2 vol % in Novec engineered fluid) treatment. The treated devices were rinsed with Novec engineered fluid and then heated in an oven at 80° C. for 30 minutes to evaporate residual oil.

### Methods—Microgel Fabrication

Freeze-dried GelMA was dissolved in DPBS, containing a photoinitiator (LAP at a final concentration of 0.1% w/v), at 40° C. to prepare a 5% w/v aqueous GelMA solution. A mixture of Novec engineering fluid, containing 2% v/v Pico-Surf™ surfactant, was used as the oil phase for the small and medium droplets, and a 0.5% v/v surfactant in the same oil was used for the large droplets. The droplet fabrication system was kept at around 35-40° C. using a space heater. Then, droplets were maintained at 4° C. overnight to form physical crosslinked microgels. Microgels were then photocrosslinked via light exposure (wavelength=395-400 nm, intensity=15 mW cm⁻²) for 5 min, followed by adding an equal volume of PFO (20% v/v in Novec engineered fluid), vortexing for 5 s, and centrifuging at 300×g for 15 s to remove the oil and surfactant. To ensure no residual oil or surfactant remained, the microgel suspension was rinsed with an equal volume of DPBS, vortexed, and centrifuged at 300×g for 15 s.

### Methods—Cell Culture

NIH/3T3 mouse fibroblast cells, primary HUVEC, or MSC were cultured in DMEM (supplemented with 10% v/v FBS and 1% v/v antibiotics), EGM-2, or complete mesenchymal stem cell growth media, respectively. The medium was refreshed every other day, and the cells were passaged when they reached 80% confluency, typically twice a week. A standard cell culture incubator (Eppendorf, Hamburg, Germany) was used to culture cells under a 5% v/v carbon dioxide (CO₂) atmosphere at 37° C. The cells were trypsinized (detached from the culture dish) using a 0.25% trypsin-EDTA solution, followed by counting using an automated cytometer (Countess 2, ThermoFisher Scientific, MA, USA).

### Methods—BHS and Cell Spheroid Formation

To form BHS without geometric constraints, crosslinked GelMA microgels were packed at 3000×g and then pipetted into a petri dish (60 mm) using positive displacement pipette (MICROMAN E M100E, Gilson Company, Inc., OH, USA). To ensure similar total surface area, 10, 27, or 50 μL of small, medium, or large packed microgel suspension was used. Cell suspension (3 million cells per petri dish) was also added in 5 mL of media, resulting in a cell concentration of 600,000 cells mL⁻¹. The microgels and cells were mixed by gentle pipetting. To form BHS under geometric constraints, GelMA microgels packed at the similar condition mentioned above. Then, microgels and cells were mixed, while maintaining a constant cell density of 600,000 cells mL⁻¹ and varying the microgel amount according to their sizes, 2, 5.4, or 10 μL mL⁻¹ for small, medium, or large, respectively. The microgel-cell suspensions were gently pipetted and transferred into disposable pipetting reservoirs. Then, 200 μL of the mixture was added to each well of a U-bottom 96-well plate. The culture was maintained for up to one week, with the media being refreshed daily. Cell spheroid is formed similar to BHS, but microgel-free.

### Methods—Analysis of BHS and Cell Spheroid Formation

A CytoSMART Lux2™ cell imaging microscope (CytoSMART Technologies, Netherlands), equipped with a 10× objective, was used to image BHS formation (unconstraint) over time. Images were acquired at 5-min intervals for 72 h and used to determine morphological and kinetic parameters. The area of each BHS was calculated using the ImageJ software (Fiji, version 1.54f, NIH, MD, USA). The attachment of the cells to both microgels and other cells during BHS and cell spheroid formation were assessed by tracking them using a custom-written Mathematica code (Wolfram Mathematica, version 13.3, Wolfram Research, IL, USA). BHS formation in the U-bottom plates (i.e., constraint geometry) were imaged using Incucyte® S3 Live-cell Analysis system (version 2022A, Sartorius, Germany) at Sartorius cell culture facilities, the Pennsylvania State University, with brightfield, and green-fluorescence protein (GFP) channels. Images acquired every 30 min for 5 days using a 4× objective and analyzed using the Incucyte® spheroid analysis software module (version 2022A, Sartorius, Germany).

### Methods—Microgels Tracking

Microgel particles were tracked using a custom Python script using the trackpy library. The stacked images were loaded, and particles were initially detected in the first frame. The parameters for particle detection, including diameter and minimum mass threshold, were optimized based on the microgel sizes to ensure accurate identification. Tracking of particles was performed frame by frame. The trajectories were visualized, and short-lived tracks persisting for fewer than 10 frames were filtered out to ensure reliability in the subsequent analysis.

### Methods—Quantification and Analysis of Single Cells in Aggregate Formation Kinetics

The attachment of the cells to both microgels and other cells during BHS and cell spheroid formation was assessed by tracking them using a custom-written Mathematica code (Wolfram Mathematica, version 13.3, Wolfram Research, IL, USA). For each microgel size, an exponential decay function was fitted to the number of individual cells over the initial 10 h. This fitting used the exponential decay model as

$$N ( t ) = {N}_{0} {e}^{\frac{- t}{τ}} ,$$

where N(t) is the number of remaining individual cells at time t, N₀ is the initial number of cells, and τ is the characteristic decay time.

### Methods—Porosity Characterization

BHS were formed in U-bottom wells (constraint) for 3 days, followed by incubation in a FITC-dextran solution (Mw≈2 MDa, 30 μM in DPBS) for 10 minutes to fill voids. Porosity was determined from 3D Z-stacked images (volume of interest: 149.66×149.66×68.21 μm³ in X, Y, and Z, respectively) acquired using a Leica STELLARIS 5 confocal microscope (Leica Microsystems, Germany). The LAS X (version 5.0.3, Leica Microsystems, Germany) software calculated void volume fractions by comparing stained interstitial space against total volume.

### Methods—Cell Staining Procedures

Cells were visualized using fluorescent staining. BHS and cell spheroid fixed with 4% paraformaldehyde for 2 h at room temperature, followed by permeabilization with 0.2% Triton X-100 in DPBS for 2 h. Then, aggregates were washed with DPBS at least five times, each for 10 min. For actin filament staining, Phalloidin Alexa Fluor 488 was applied (1:40 volume ratio in DPBS) and incubated overnight in darkness at 4° C. Nuclear staining was achieved using DAPI (1:1000 volume ratio in DPBS) for 2 h. Live cell labeling was performed using CellTracker Green CMFDA and Red CMTPX, according to the manufacturer's protocol on 2D cultured cells. Additionally, live cells nuclei stained with Hoechst (1:2000 volume ratio in PBS) for 10 min.

### Methods—Scanning Electron Microscopy (SEM)

The surface morphology of aggregates was investigated using a scanning electron microscope (Quanta 250 ESEM, Thermo-Scientific, OR, USA) at the materials characterization lab at the Pennsylvania State University. Cell spheroid or BHS were formed in 3 days, then fixed in 4% v/v of paraformaldehyde for 3 h. The fixed samples were rinsed at least five times with DPBS and subsequently immersed in a gradient of ethanol solutions with concentrations ranging from 15% to 100% (v/v in milli-Q water). The samples were then dried using a critical point dryer (CPD300, Leica EM, Germany) to ensure complete removal of any fluid. Finally, the samples were sputter-coated with iridium (thickness ˜2-5 nm, Emitech K575 Turbo sputter coater, E.M. Technologies, UK) and imaged with a beam current of 91 pA under an accelerating voltage of 5 keV, using an Everhart-Thornley detector (ETD) in Secondary Electron (SE) mode.

### Methods—Cell Metabolic Activity Assessment

The cell metabolic activity solution was prepared by adding Presto Blue™ cell viability solution to DMEM (serum-free) in a 1:9 volume ratio. The BHS cultured on a planar rigid substrate (unconstraint) were removed using a cell scraper, then moved into a 15 mL centrifuge tube. The tube was centrifuged for 5 minutes at 300×g. After discarding the supernatant, 3 mL of metabolic activity solution was added to the centrifuged BHS. The tubes were wrapped in aluminum foil and incubated for 4 h in a cell culture incubator at 37° C., with 5% CO₂. Then, the tubes were re-centrifuged at 300×g for 5 minutes and the supernatant was collected for analysis. Fluorescence intensity was recorded using a microplate reader (Tecan Infinite M Plex, Switzerland) at 560 nm excitation and 590 nm emission. The metabolic activity of BHS formed in U-bottom well-plates (constraint) was measured using a metabolic activity solution of Presto Blue™ cell viability solution mixed with equal volume of serum-free DMEM. Then, 50 μL of this stock solution was added to each well. The plate incubated at 37° C., with 5% CO₂, for 4 h, followed by analysis using the aforementioned device/condition.

### Methods—Cell Viability Assessment

Using a cell scraper, all cells, microgels, and/or formed aggregates were detached from the petri dish and transferred to a centrifuge tube. The tubes were centrifuged at 300×g for 5 min, followed by discarding the supernatant. Cell viability within the BHS or cell spheroid was assessed using a two-color fluorescence LIVE/DEAD™ cell imaging kit. The imaging solution made from Calcein AM as live cell indicator and BOBO-3 Iodide for staining dead cells was prepared according to the manufacturer protocol, followed by adding 1 mL into each centrifuge tube and resuspending via gentle pipetting. The centrifuge tubes were then wrapped in an aluminum foil and placed under the biosafety cabinet at room temperature for 30 min and imaged using the Leica DMi8 fluorescence microscope (THUNDER imaging systems, Leica Microsystems, Germany). The live cells channel was set to 470 nm excitation and 510 nm emission wavelengths. The dead cell channel was set to 550 nm excitation and 610 nm emission wavelengths. Images analyzed using ImageJ software (Fiji, version 1.54f, NIH, MD, USA), and cell viability was reported as the number of live cells, over total number of cells.

### Methods—Angiogenic Sprouting Assay

Sprouting assays were performed using HUVEC cell spheroids or BHS. Matrigel was thawed overnight at 4° C., coated onto a 48-well plate, and cured at 37° C. for 4 h. Assemblies were cultured on top of the Matrigel for 3 days, stained with the two-color fluorescence LIVE/DEAD™ cell imaging kit, and imaged using the Leica DMi8 fluorescence microscope (THUNDER imaging systems, Leica Microsystems, Germany).

### Methods—Fusion Test

BHS or cell spheroid aggregates were prepared in U-bottom wells (constraint) for 3 days. Four aggregates of the same type (either BHS-S, BHS-M, BHS-L, or cell spheroid) were transferred and placed in proximity within a U-bottom well. Media was refreshed daily. Brightfield images were captured every day using an EVOS™ XL Core microscope (Thermo Fisher Scientific, MA, USA). Area of fused aggregates was measured using ImageJ software (Fiji, version 1.54f, NIH, MD, USA), and results were reported accordingly.

Methods—Formation of mm-Scale Tissue-Like Structures

BHS were cultured on a non-constraining, rigid planar substrate for 3 days. Subsequently, these aggregates were detached using a cell scraper and passed through a cell strainer (40 μm pores) to remove unbound cells and debris, ensuring retention of BHS on the strainer due to their size. The collected aggregates were then placed into a custom-designed PDMS cylindrical mold (12 mm diameter, 3 mm height), ensuring close contact among them. Following an additional 3 days of culturing with daily media refreshment, a large mm-scale scaffold was formed, which could be readily removed using a spatula.

### Methods—GelMA Bulk Hydrogel Scaffold Fabrication

Bulk GelMA hydrogel scaffolds were fabricated using a two-step crosslinking process, mirroring the method for microgel fabrication, to ensure that they share similar physicochemical properties. Scaffolds were prepared by dissolving GelMA in a 0.1% w/v LAP solution in DPBS at a concentration of 5% w/v at 40° C. until GelMA dissolved completely. This homogeneous GelMA solution was poured into cylindrical acrylic molds (diameter=10 mm, height=1 mm). To prevent dehydration, molds were placed in a dark, custom-built humidity chamber. After cooling at 4° C. overnight for physical crosslinking, hydrogels underwent photocrosslinking under light (wavelength=395-400 nm, intensity=15 mW cm⁻²) for 5 min, resulting in stable bulk hydrogel scaffolds.

### Methods—Compression Test

The mechanical properties of GelMA bulk hydrogel scaffolds were assessed using an Instron mechanical tester (Model 5542, Instron Corporation, MA, USA). Samples were pre-incubated at 37° C. for 2 h, punched into dimensions of 8 mm in diameter and 1 mm in height, then subjected to uniaxial compression at a controlled displacement rate of 1 mm min⁻¹. Testing continued until 70% strain or sample failure. Stress-strain curves were recorded, and the compressive modulus was calculated from the initial linear region, between 0 and 10% strain.

### Methods—Viscoelastic Properties Assessment

Viscoelastic properties of GelMA hydrogel scaffolds and tissue-like structures were examined using a rotational rheometer (AR-G2, TA Instruments, DE, USA) with parallel plates (8 mm top plate and 20 mm bottom plate, both sandblasted stainless steel). Samples were punched to match the upper plate diameter for full coverage, then were incubated at 37° C. for 2 h. Rheological tests (performed at 37° C.) included an oscillatory amplitude sweep from 0.1% to 100% strain at a constant frequency of 1 rad s⁻¹ to identify the linear viscoelastic region (LVR), and a subsequent frequency sweep at 0.1% strain, from 0.1 to 100 rad s⁻¹. The storage modulus (G′) and loss modulus (G″) were evaluated to assess the sample viscoelasticity.

### Methods—Statistical Analyses

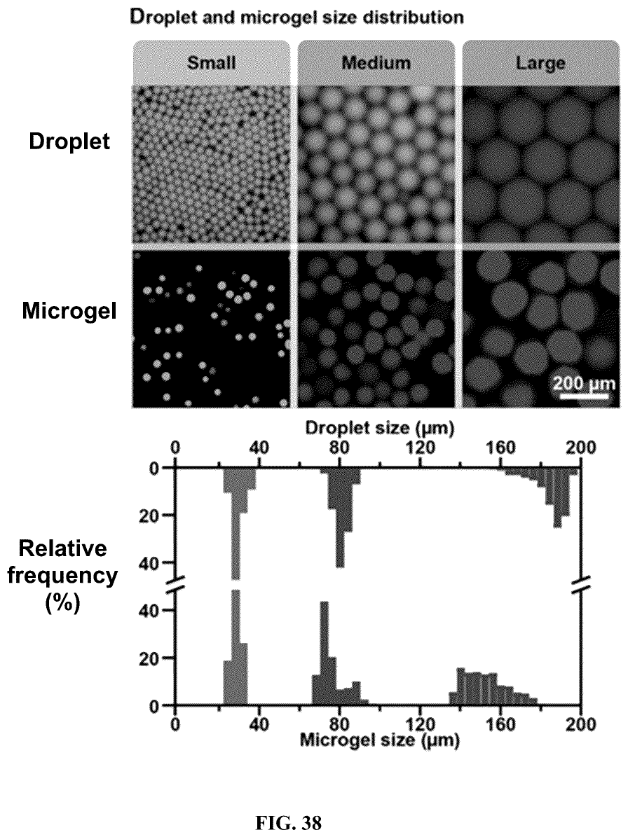

Experiments were conducted with at least three repeats. Data points in all figures represent independent repeats, except for FIG. 38, where all technical replicates within the ≥3 independent repeats are shown to represent the data distribution. Normally distributed data were analyzed using t-tests, or either ordinary or repeated measures (RM) one-way/two-way analysis of variance (ANOVA), with significance determined by Tukey's post-hoc multiple comparison test. Non-normally distributed data were assessed using the Kruskal-Wallis test followed by Dunn's post-hoc multiple comparison test. All statistical analyses were performed using GraphPad Prism (version 9.5.0). Groups with p-value below 0.05 were considered significantly different, indicated by *p<0.05, **p<0.01, ***p<0.001, ****p<0.0001.

### Results and Discussion

Cell Adhesion and Migration Device Drive Snowballing-like Assembly of BHS

Cells are provided with microscale ECM-mimetic substrates, gelatin methacryloyl (GelMA), a protein-based biopolymer, which is synthesized via reacting gelatin and methacrylic anhydride. GelMA is a photocrosslinkable, biodegradable biopolymer with tunable physicochemical properties. Upon light exposure, a photoinitiator (PI)-containing GelMA biopolymer solution forms a chemically crosslinked hydrogel, which is stable at physiological conditions (e.g., 37° C.). GelMA contains amino acid sequences, such as RGD peptides, that promote cell adhesion by interacting with integrins on the cell surface.

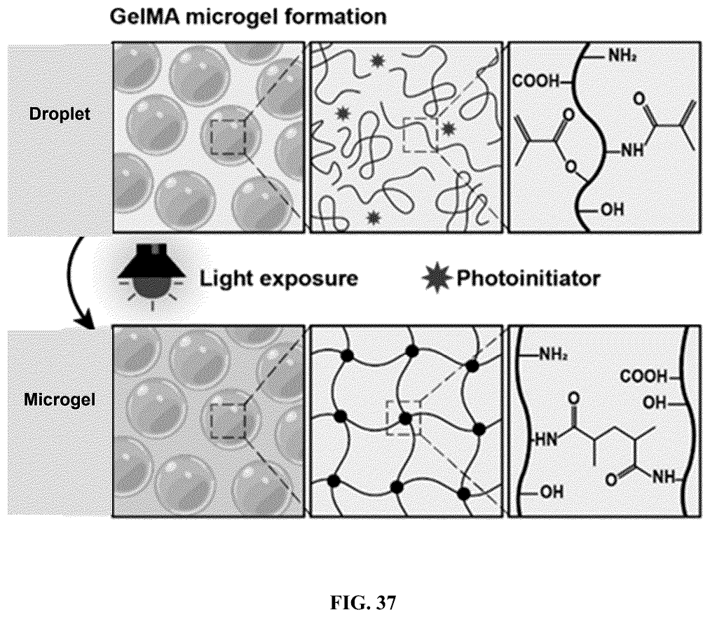

GelMA microgels with a controlled size are fabricated using step-emulsification microfluidic devices, which provide ECM-mimetic substrates for cell adhesion. By adjusting the step sizes of the microfluidic devices, droplets of varying diameters are produced according to an established method. These droplets are then converted to stable microgels through the free-radical photopolymerization of vinyl groups (FIG. 37). FIG. 38, shows the GelMA droplets, corresponding crosslinked microgels, and their size distribution. The average droplet diameter was 29±3 (small), 81±4 (medium), or 183±11 μm (large). Microgels undergo size reduction during photocrosslinking to 28±2, 76±6, and 152±10 μm for small, medium, and large, respectively. This reduction in microgel size post-photocrosslinking originated from the GelMA network densification, which leads to water expulsion and volume decrease. The sizes of these microgels are selected to approximate or exceed the sizes of NIH/3T3 fibroblast cells (measured diameter ˜15±2 μm in a cell suspension).

Mixing the microgels with the NIH/3T3 murine fibroblast cells on a planar non-adhesive substrate prompts cell-cell and cell-microgel interactions. As schematically shown in FIG. 39, cell-cell interactions are facilitated by homophilic cadherin-cadherin binding and integrin-mediated binding to ECM components, such as fibronectin recycled by the cells. Integrin-mediated binding between cells and GelMA microgels, specifically RGD peptides and fibronectin, drives cell-microgel interactions. FIG. 40 shows the co-culture of microgels and fibroblast cells. The top row schematically represents the three-step assembly process: (i) initial mixing of cells and microgels, (ii) early-stage attachment of cells to microgels because of the adhesive moieties on GelMA, and (iii) the formation of two distinct aggregate types. The bottom row shows the pseudo-colored microscopy images of actual microgel-free cell spheroids, arising from cell-cell interactions, and BHS, which form through cell-microgel assembly.

### Microgel Size Regulates the Snowballing Kinetics and Terminal BHS Size

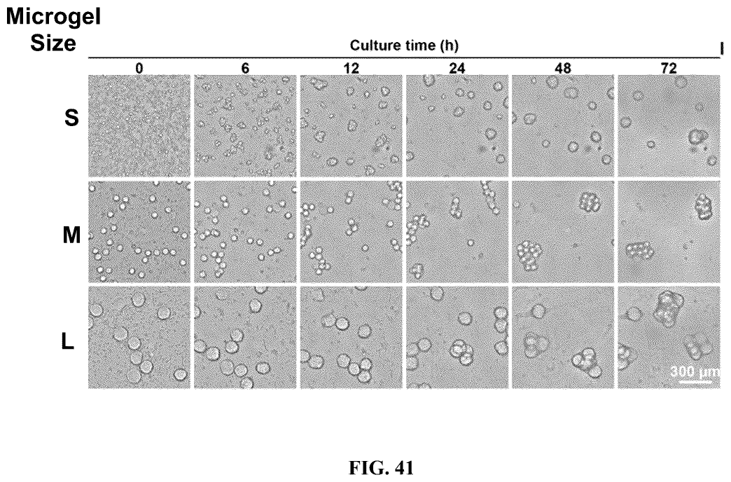

FIG. 41 shows the culture of fibroblast cells with microgels of three different sizes on a planar surface for 72 h, resulting in BHS formation. As cell-microgel interactions drive the aggregation and BHS formation, which depend on the accessible microgel surfaces, the total microgel surface area was maintained nearly constant in all the experiments. The total projected areas for small, medium, and large microgel suspensions are 13.7±0.9%, 14.8±3.0%, and 13.5±5.0%, respectively, showing no statistically significant differences. BHS formation occurs primarily within 24 h, but the aggregates continue to move, connect, and merge upon contact with much slower rates. Interestingly, we observe that cell spheroids are simultaneously formed in microgel-free regions, which may subsequently adhere to BHS upon contact. Control groups consisting of only microgels or cells are also monitored over time. Cell-free microgels do not undergo any significant movement or aggregate formation, showing cell mobility as the exclusive driving force for the aggregation. In contrast, the control comprising only cells undergo cell spheroid formation over time.

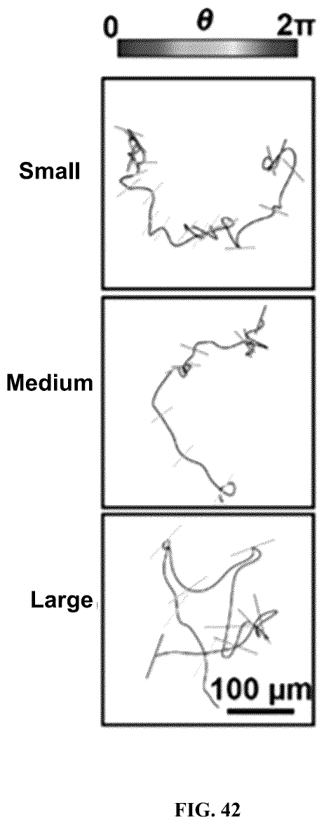

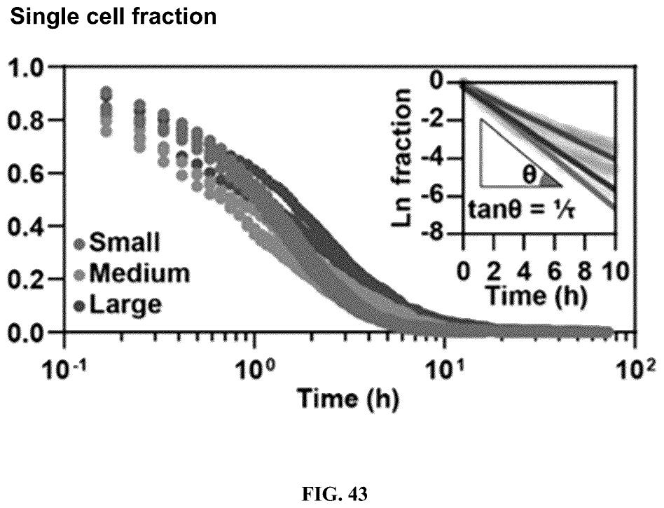

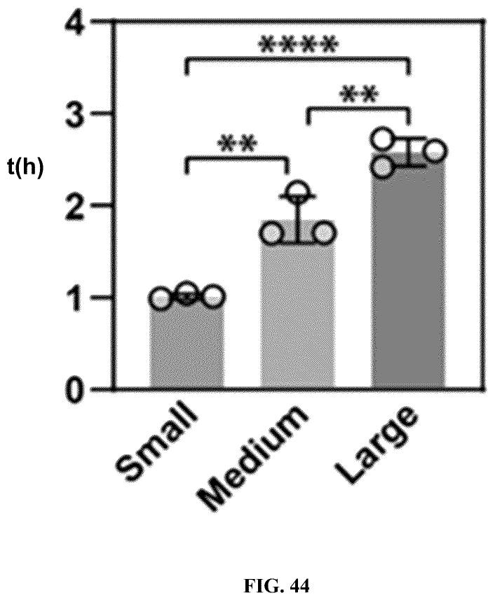

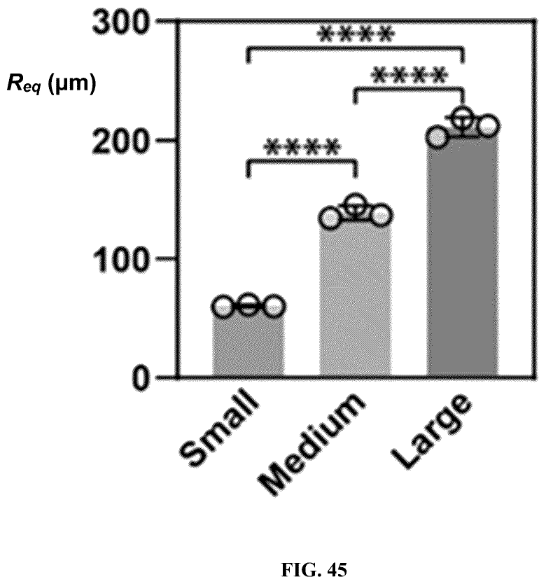

Our experimental observations indicate that the differential affinities between cells, microgels, and substrate are the primary drivers of BHS formation. The cells exhibit a higher integrin-mediated affinity for microgel surfaces than the untreated substrate. Since microgels are not self-adherent, the cells act as essential binding agents in the cell-microgel aggregation process (bio-glue). As a cell migrates from the substrate to microgels, its adhesion and contractility drive both translational and rotational motions of the growing aggregate, resembling to a snowballing process, facilitating the 2D to 3D transition of cell-microgel assemblies. Such rotational movement is evidenced by the dynamically changing angle of the line vector connecting two constituent microgels within the same spheroid, relative to the fixed coordinates of the culture plane, as shown in FIG. 42. To track the kinetics of BHS formation, we quantify the ratio of remaining individual cells due to aggregation depletion over time, as shown in FIG. 43. We observed a rapid exponential decline in the number of individual cells, dropping to <1% within approximately 10 h in all cases, indicating a nearly complete integration of all cells into aggregates. Under the same total surface area (Sₜₒₜ) of microgels, the characteristic decay time (τ) increases with microgel radius (FIG. 44), where τ is the time needed to reduce the number of individual cells to 1/e of its initial value. Furthermore, the terminal size of BHS (after 72 h) is quantified, as presented in FIG. 45, by measuring their equivalent radius R_{eq}=\sqrt{A/π}, where A denotes the projected area of an aggregate. FIG. 45 also shows that R_{eq} increases with microgel size and that the number density of stable BHS is higher for smaller microgel size. Overall, the microgel size determines the final aggregate size, which is kinetically arrested at long culture times (>10 h).

### BHS Architecture is Tailored by Geometric Constraints

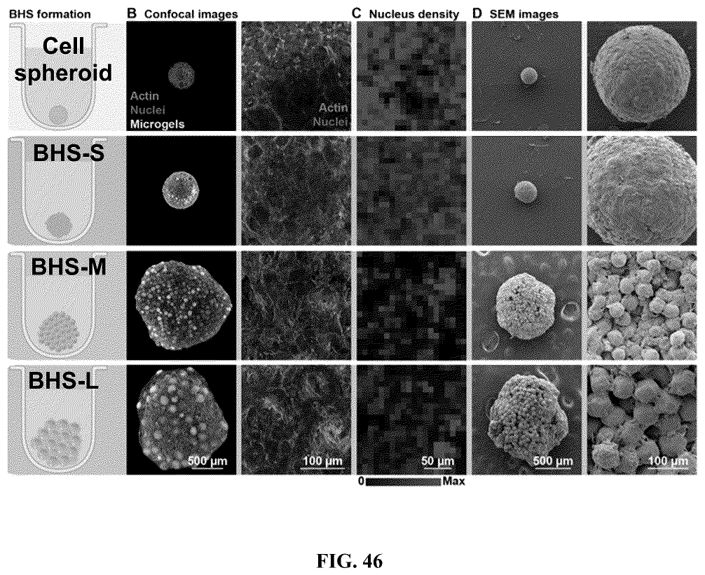

Cell-microgel culture on a flat, untreated substrate results in irregularly shaped BHS and cell spheroids with a diverse size distribution. Thus, an experimental system to enable single, large BHS formation with pre-determined cell and microgel number density is necessary to further assess microgels impact on cell behavior and aggregate morphology/porosity. In contrast to unconstraint, freely formed cell-microgel assemblies on a flat surface, BHS with a more controlled and regular shape are formed by mixing the cells and microgels in a low-attachment U-bottom well-plate. BHS-S, BHS-M, and BHS-L, or cell spheroid are formed using small, medium, and large microgels, or microgel-free cells, respectively, as schematically shown in FIG. 46. Confocal images of BHS and cell spheroids show that the cells are elongated and spread among the microgels in BHS-M and BHS-L, while in BHS-S and cell spheroids they remain more compact (FIG. 46). Moreover, FIG. 46 shows that the distance among cell nuclei is smaller in the cell spheroid and BHS-S compared with BHS-M and BHS-L, highlighting the increased cell compactness of the former aggregates.

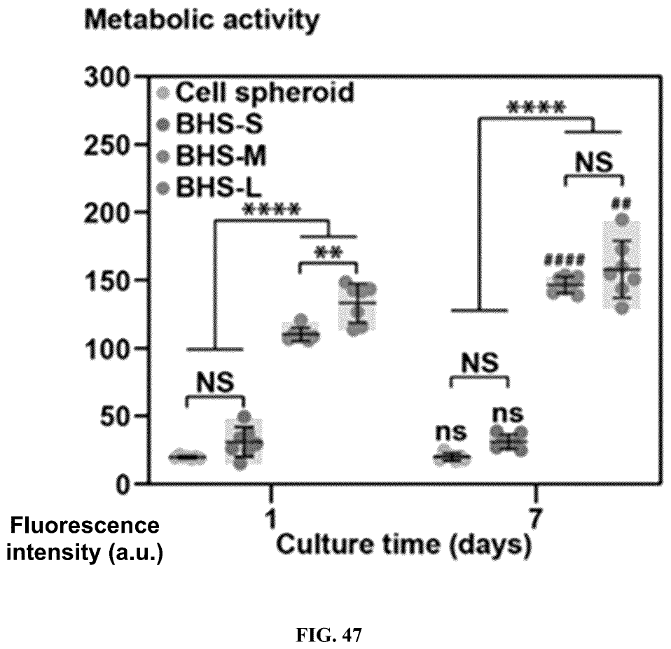

Scanning electron microscopy (SEM) images of aggregates in FIG. 46 show that cell spheroid and BHS-S are smaller and more spherical in shape compared with BHS-M and BHS-L. Compactness in cell spheroids correlates with sphericity, as the formation of cell spheroids depends on collective forces exerted on cells. In FIG. 47, we measure the metabolic activity of cells within aggregates on days 1 and 7, keeping a constant initial cell density.

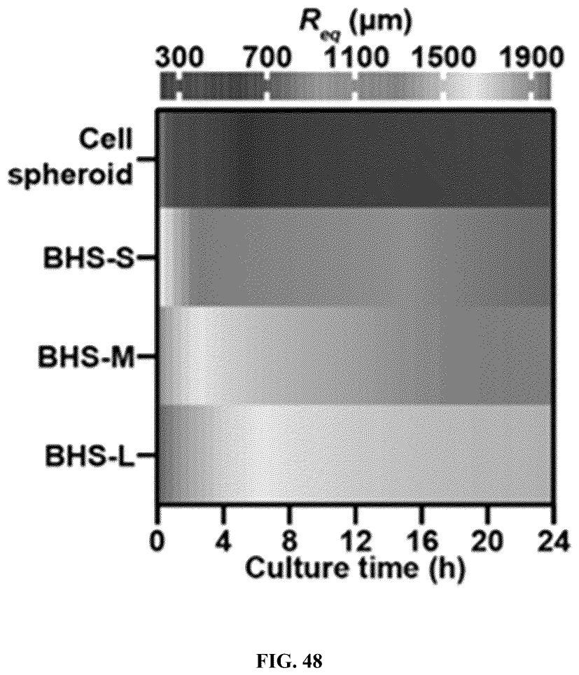

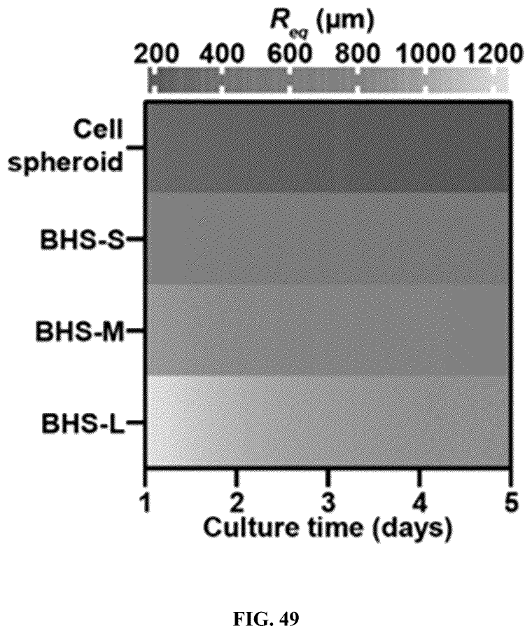

To analyze the dynamic formation of geometrically constraint cell spheroids and BHS over time, we acquired microscopic images of BHS and cell spheroid formation throughout a 5-day period. The control experiment with cell-free medium microgels did not result in any aggregate formation. Analyzing aggregate sizes over time highlights a trend: larger microgels need more time to reach a plateau in size, because at a similar cell attachment rate (FIGS. 42-44), larger microgels experience higher static friction and viscous forces due to an increased size and surface area. The most substantial BHS size change occurs within the initial 24 h (FIG. 48). The R_{eq} heatmap in FIG. 49 presents long-term size changes over five days of microgel-cell culture, showing that the change in size is microgel size dependent and eventually becomes kinetically arrested. BHS-S reached a stable size within 2 days, BHS-M required 3 days, and BHS-L took 4 days to attain size stability. Additionally, cell spheroid size was stabilized after 4 days, and the control group containing solely microgels had no size change throughout the experimental period.

Cells serve as both motors and adhesives agents in the BHS formation process; thus, we investigate their effects on BHS formation by manipulating the initial cell seeding density and monitoring BHS-M formation. Evaluations through both brightfield and fluorescence imaging show a notable deceleration in BHS formation when the cell density decreases. As the cell count decreases, there is a shift towards an extreme case resembling cell-free microgels. In these cases, the necessary driving force for assembly, namely cell-hydrogel interfacial interactions, may be compromised. Assemblies formed with fewer than 30,000 cells after a 5-day period lack sufficient cohesion, resulting in disintegration during handling, e.g., pipetting.

### BHS Formation Extends to Endothelial and Mesenchymal Stem Cells and Promote Angiogenic Sprouting

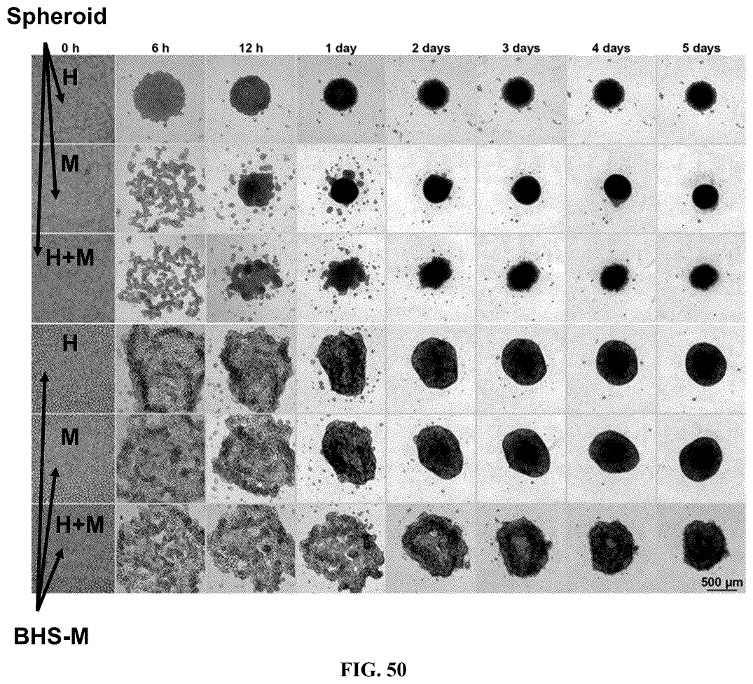

To extend our “snowballing-like” BHS formation beyond contractile NIH/3T3 fibroblasts, we investigate whether primary human umbilical vein endothelial cells (HUVECs), mesenchymal stem cells (MSCs), and their mixtures can form similarly robust 3D assemblies with GelMA microgels (FIG. 50). We formed six groups of cells-only or cell-microgel assemblies in geometrically constrained wells: (i) cell spheroids of HUVECs, MSCs, or HUVEC+MSC, and (ii) their corresponding BHS, formed by mixing each of these cell populations with GelMA microgels.

Initially (0-12 h), HUVEC-only spheroids rapidly adopt a round shape, similar to our earlier observations with fibroblasts, whereas MSC-only and HUVEC+MSC spheroids show incomplete compaction. This difference arises from robust HUVEC homophilic cell-cell adhesion (e.g., via VE-cadherin), which drives early spherical assembly. In contrast, MSC relies more on de novo ECM secretion beside homophilic contacts (e.g., via N-cadherin or Cadherin-11), resulting in slower aggregation.

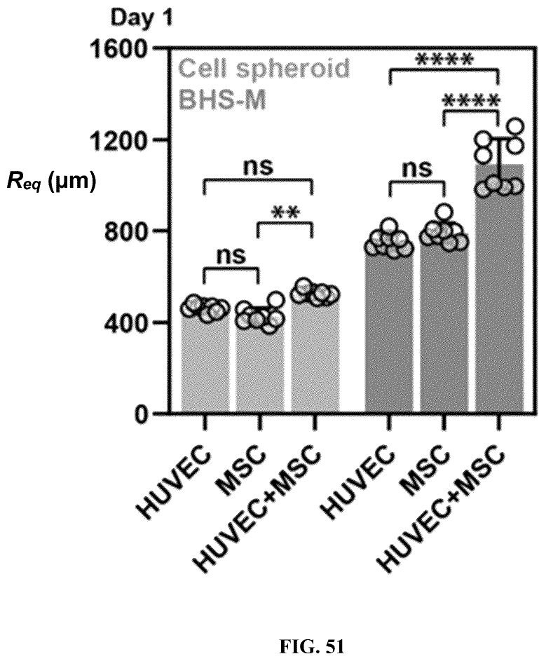

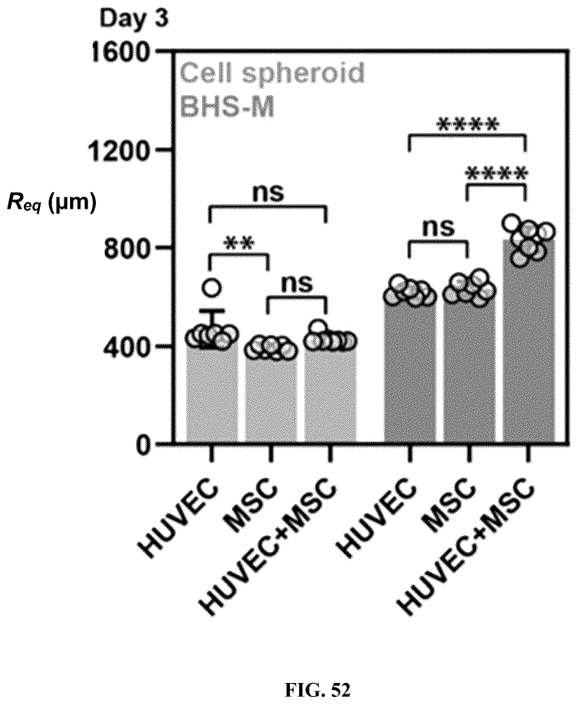

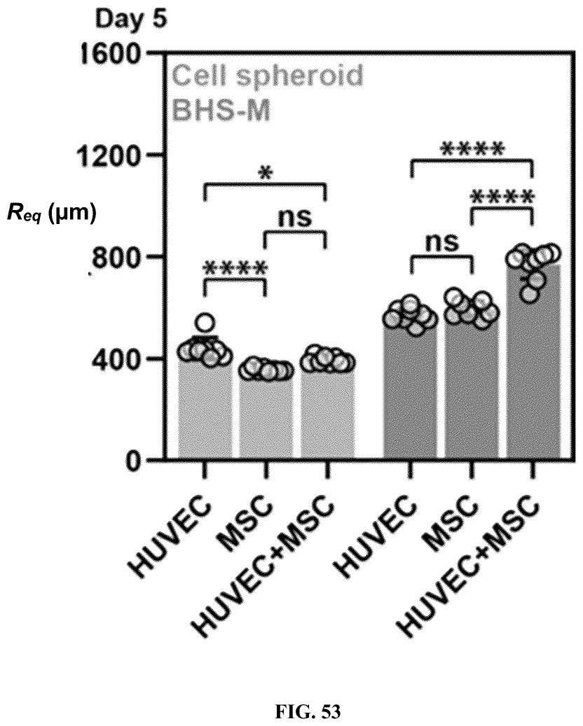

FIG. 51 captures the differences in equivalent radius (R_{eq}) on day 1: HUVEC spheroids appear as the largest among the cell-only aggregates, whereas the HUVEC+MSC BHS form the largest assembly among the BHS groups. We attribute this behavior to the heterotypic interactions between two cell types and the microgels, as well as the potential formation of early vessel-like structures—previously reported in HUVEC-MSC co-culture with GelMA—that collectively drive cell spatial reorganization which may be more voluminous. By days 3 and 5 (FIGS. 52-53), HUVEC spheroids maintain a relatively larger equivalent radius than MSC or HUVEC+MSC spheroids, likely because HUVECs, especially in pro-angiogenic media, favor hollow or less-dense spheroid architectures and secrete less ECMs than MSCs and fibroblasts. In contrast, MSC-only and HUVEC+MSC spheroids progressively densify, influenced by continued ECM deposition. Meanwhile, the HUVEC+MSC BHS persistently feature the largest size among the BHS conditions. Overall, these results indicate that BHS assembly extends beyond highly contractile fibroblast models to other cell types, such as HUVECs and MSCs, although the final size and aggregation kinetics vary.

We further evaluate the functional outcomes of these HUVEC-based assemblies by examining angiogenic sprouting. Compared with conventional HUVEC spheroids, which yield minimal outgrowth because of dense cell packing and inhibited nutrient diffusion, HUVEC BHS undergo substantially more pronounced vascular-like branching. This likely results from the microgel-mediated porosity that supports nutrient transport and cellular metabolism, thereby promoting angiogenic sprouting in vitro.

### BHS as a Versatile Building Block for Large Tissue-Like Structures In Vitro

Current hydrogel-based in vitro tissue models larger than the molecular diffusion limit (e.g., hundreds of microns) require perfusable blood vessels or channels to overcome the diffusion limit of oxygen and metabolite and maintain cell viability. Here, we assess BHS as building blocks for developing mm-scale tissue-like structures without requiring blood vessels. A critical criterion in developing large tissues in vitro is cell viability throughout the construct. We have shown that BHS made up of medium microgels have significantly higher cell viability and metabolic activity compared with the microgel-free cell spheroid.

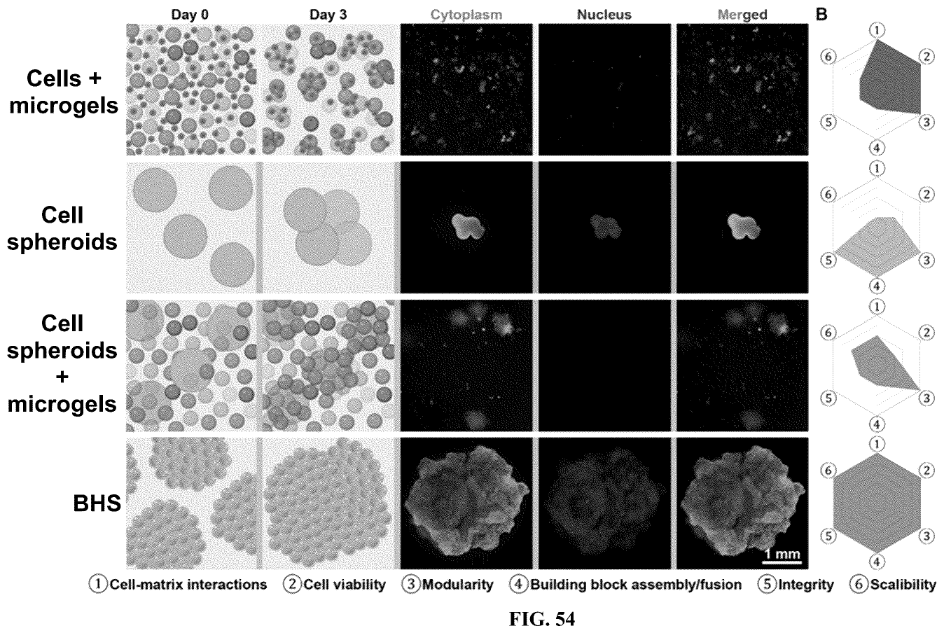

In FIG. 54, different building blocks, cells, microgels, cell spheroid, and BHS are used to generate tissue-like structures within 72 h. For a better comparison, four aggregates (cell spheroid/BHS) or the equivalent quantities of their components (cells/microgels) were compared. The initial configuration entails a mixture of cells and microgels, allowed to form BHS on a planar substrate without any geometric constraints. The design variables were cell density (480,000 cells, equivalent to 4 cell spheroids/BHS), microgel concentration (108 μL, equivalent to 4 BHS), and culture time (72 h), using which formed aggregates had R_{eq} of 139±82 μm. Changing the variables may results in formation of different sizes of aggregates, which have been reported. The second configuration involved fusing cell spheroid on a flat untreated surface, which yielded constructs with R_{eq} of 330±18 μm. While cell spheroids have been used as building blocks for tissue engineering scaffolds and biofabrication, their inherent limitations, such as hypoxic core formation and compactness/density, render the fabrication of viable large tissue-like structures non-trivial, unless they are perfusable or vascularized.

The third configuration involves cell spheroid and microgels, which have been reported for tissue engineering applications. For formation of a tissue-like structure, the connectivity among microgels and cell spheroid is crucial, which is regulated by the ratio of cell spheroid to microgel size/density. In our experiments, using four cell spheroids leads to the microgels accumulation on the surface of the cell spheroid. This microgel coating impeded cell spheroid fusion blocking the formation of constructs at the mm scale. Finally, BHS were allowed to contact, resulting in mm scale (R_{eq}=1339±119 μm) fused tissue-like structures that could be pipetted, indicative of the unique capacity of BHS for large-scale tissue fabrication with sizes reaching up to 4 mm in diameter in this work. All together, radar plots in FIG. 54 compare BHS as building blocks for fabricating large tissue-like constructs in vitro with cell/microgels, cell spheroid, and cell spheroid/microgels based on cell-matrix interactions, scalability, structural integrity, cell viability/metabolic activity, modularity and building block fusion for tissue formation.

### Example 4: Granular Aerogel Scaffolds

Materials: Gelatin type A from porcine skin (gel strength ˜300 g Bloom), methacrylate anhydride (MAA), trichloro(1H,1H,2H,2H-perfluorooctyl)silane (F-silane), lithium phenyl-2,4,6-trimethylbenzoylphosphinate (LAP), trypsin-ethylenediaminetetraacetic acid (EDTA) solution (0.25%), citrate buffer (pH=6.0), TWEEN® 20, Parafilm® M sealing film, and bovine serum albumin (BSA) were supplied by MilliporeSigma, MA, USA. Ultrapure water (electrical resistivity ˜18.2 MΩ cm at 25° C.) was generated by Milli-Q water purification system (Millipore Corporation, MA, USA). Phosphate-buffered saline (PBS), Dulbecco's phosphate-buffered saline (DPBS) without calcium (Ca²⁺) and magnesium (Mg²⁺), Dulbecco's Modified Eagle Medium (DMEM), fetal bovine serum (FBS), and penicillin-streptomycin (10,000 U mL⁻¹ penicillin and 10,000 μg mL⁻¹ streptomycin) were purchased from Gibco, MA, USA. T-75 cell culture flasks, 24 and 96 well non-treated plates, and centrifuge tubes (15 mL and 50 mL) were supplied by CELLTREAT® Scientific Products, MA, USA. VWR® vacuum filtration systems (0.2 μm), VWR® VistaVision™ microscopic slides, KIMWIPES™ delicate task wipers, VWR® disposable Petri dishes, and razor blade were supplied by VWR, PA, USA. PrestoBlue™ high-sensitivity (HS) cell viability reagent, live/dead cell viability assay kit (calcein acetoxymethyl (AM) and BOBO-3 iodide), Hoechst 33258, Hoechst 34580, and CellTracker™ Green 5-chloromethylfluorescein diacetate (CMFDA) were provided by Invitrogen, MA, USA. Spectra/Por 4 dialysis tubing (molecular weight cut-off=12-14 kDa) was supplied by Spectrum Laboratories Inc., TX, USA. Diamond® white glass charged slides were provided by Globe scientific, NJ, USA. Syringes with BD Luer-Lok™ Tip were procured from BD, NJ, USA. Novec™ 7500 Engineered Fluid was purchased from 3M, MN, USA. Pico-Surf© (5 vol % in Novec™ 7500 Engineered Fluid) was provided by Sphere Fluidics, Cambridge, UK. 1H,1H,2H,2H-perfluorooctanol (PFO) was supplied by Alfa Aesar, MA, USA. NIH/3T3 murine fibroblast cells were purchased from ATCC, VA, USA. Ethanol was supplied by Decon Labs, Inc., PA, USA. Loxicom® meloxicam solution (5 mg mL¹) for injection was procured from Norbrook Laboratories, KS, USA. Isoflurane was purchased from Akorn, Inc., IL, USA. Carbon dioxide (CO₂) was provided by Lindle, CT, USA. Chlorhexidine® scrub was purchased from Aspen Veterinary Resources LTD., CO, USA. Paraformaldehyde (PFA) and glucose were purchased from Thermo Scientific, IL, USA. Sodium chloride (NaCl, lab grade) was purchased from Flinn Scientific, IL, USA. Surgipath® Paraplast X-tra was provided by Leica Biosystems (Germany). Live Cell Imaging Solution (1×), recombinant Alexa Fluor® 488 anti-cluster of differentiation 31 (CD31) antibody, recombinant Alexa Fluor® 647 anti-CD68 antibody, Fluoroshield mounting medium with 4′,6-diamidino-2-phenylindole (DAPI) were supplied by Abcam, CA, USA. CD206 monoclonal antibody (2A6A10), CoraLite® Plus 488, CD11b monoclonal antibody (M1/70), eFluor™ 660, eBioscience™, alpha-smooth muscle actin (α-SMA) monoclonal antibody (1A4), eFluor™ 570, eBioscience™, CD86 monoclonal antibody (GL-1), chicken anti-rat IgG (H+L) cross-adsorbed secondary antibody, and Alexa Fluor™ 647 were purchased from ThermoFisher, MA, USA. Macrosette® processing/embedding cassettes with cover were supplied by Ted Pella Inc., CA, USA, and 96-well flat clear bottom black polystyrene microplates were purchased from Corning, NY, USA. Acrylic sheets were provided by Astra Products Inc., NY, USA, and 50 mm glass bottom dishes (No. 0 coverslip, 30 mm glass diameter, uncoated) were purchased from MatTek Life Sciences, MA, USA. Hematoxylin 560, Alcoholic Eosin Y 515, Blue Buffer 8, Define, and xylene substitute were purchased from Lecia Biosystems, IL, USA.

Methods—GelMA Synthesis: Preheated DPBS at 50° C. with a volume of 200 mL was mixed with 20 g of gelatin. After gelatin was completely dissolved, 16 mL of MAA was slowly dripped into the solution to reach a final concentration of 8% v/v. By covering the reaction container using aluminum foil, the solution was protected from light. After 2 h of reaction at 50° C., 400 mL of 50° C. DPBS was added to the reaction container to stop the reaction. Unreacted MAA and byproducts were separated from the diluted solution using the dialysis membrane tubing against 40° C. ultrapure water for 10 days. The dialyzed solution was then sterile filtered using 0.2 μm vacuum filters, and the purified solution was frozen at −80° C. for 3 days. To obtain solid GelMA, the frozen solution was lyophilized at ˜0.017 mbar for 3 days using a Labconco™ Freezone™ 4.5 L benchtop freeze dryer (Labconco, MO, USA). The final product was maintained at 2° C. for further use.

Methods—Hydrogel microparticle fabrication: High-throughput step emulsification microfluidic devices with the step size of ˜8, ˜27, or ˜60 μm were fabricated to generate small, medium, or large GelMA droplets, respectively. The aqueous phase was prepared by dissolving LAP photoinitiator (PI) in DPBS to make a 0.1% w/v PI solution. Lyophilized GelMA was then dissolved in the PI solution at 40° C. to make a 10% w/v aqueous GelMA solution, which was used as the aqueous phase in the step-emulsification microfluidic device. For the oil phase, varying concentrations of Pico-Surf® surfactant in Novec™ 7500 Engineered Fluid were used. For the small and medium droplet fabrication, a surfactant concentration of 2% v/v in oil was used, and for the large droplets, the concentration was lowered to 0.5% v/v. Each phase was loaded in a 10 mL syringe, and its flow rate was controlled by a syringe pump (PHD 2000, Harvard Apparatus, MA, USA). For the small, medium, or large droplet fabrication, the aqueous phase flow rate was set at 20, 80, or 150 μL min⁻¹, respectively, and the oil phase flow rate was set at 50, 120, or 300 μL min⁻¹, respectively. The microfluidic devices and the aqueous phase syringe were maintained at ˜40° C. using a hair dryer and a space heater to prevent GelMA physical gel formation inside the device. The droplet fabrication setup was protected from the light by dimming the ambient light and shielding it using aluminum foil. The fabricated droplets were physically crosslinked at 4° C. for at least 24 h to yield near-uniform GelMA hydrogel microparticles suspended in oil.

Methods—GHS fabrication: Oil and surfactant were removed from the physically crosslinked hydrogel microparticles using a solution of PFO in Novec™ 7500 Engineered Fluid (20% v/v) at a 1:1 volume ratio of oil:hydrogel microparticle suspension centrifuged at 100×g for 15 s. The mixture was then vortexed and centrifuged for 15 s at 300×g. The excess oil and surfactants were discarded, followed by resuspending the microparticles in the PI solution (0.1% w/v LAP in DPBS) at a 1:1 volume ratio. The suspension was centrifuged again for 15 s at 300×g, and any remaining oil and surfactant were removed. A centrifugal force of 5220×g was then used to jam 400 μL of hydrogel microparticles for 15 s, and the supernatant was discarded. The jammed hydrogel microparticles were transferred into laser-cut acrylic molds using a positive displacement pipette (Microman E M100E, Gilson, OH, USA). Jammed hydrogel microparticles inside the molds were then exposed to light (UV LED Flood Light, QUANS, China, source power=20 W, wavelength=395-405 nm, and intensity=15 mW cm⁻²) for 120 s. The covalent interlinking of jammed small, medium, or large microparticles yielded S-GHS, M-GHS, or L-GHS, respectively.

Methods—Bulk hydrogel scaffold fabrication: A PI solution (0.1% w/v LAP in DPBS) was prepared and preheated to 40° C. To make a 10% w/v GelMA solution, lyophilized GelMA was dissolved in the preheated PI solution. The GelMA solution was then pipetted into laser-cut acrylic molds, followed by transferring the molds to a Petri dish, containing hydrated Kimwipes to maintain the humidity constant. The chamber was then covered with aluminum foil to prevent light exposure and maintained at 4° C. for 24 h to enable GelMA physical crosslinking. Physically crosslinked GelMA hydrogel scaffolds were then exposed to light (wavelength=395-405 nm and intensity=15 mW cm⁻²) for 120 s to form bulk hydrogel scaffolds.

Methods—GAS and bulk aerogel scaffold fabrication: Ethanol solutions with varying concentrations (30, 50, 60, 70, 80, 90, or 95% v/v) were prepared in ultrapure water. GHS or bulk hydrogel scaffolds were initially transferred to a container filled with 30% v/v ethanol and incubated for 10 min at room temperature. Scaffolds were then incubated in solutions with an ascending ethanol concentration at 10-min intervals. After the last step (95% v/v ethanol in ultrapure water), the scaffolds were incubated in 100% ethanol for 30 min at room temperature. This step was repeated two more times, each time replacing the ethanol with fresh 100% ethanol. After the solvent exchange step, scaffolds underwent SCD using a critical point dryer (CPD300, Leica Microsystems, Germany). At each run, the stirrer was at 50% speed, and CO₂ influx speed in the pressure chamber was set to slow. The delay time before starting the exchange process was 120 s, and the exchange speed was set to 1 for 18 cycles. The heating speed was slow, and the gas out speed was adjusted to 20% of its normal speed. The chamber temperature during the process was heated up to 40° C. to ensure that CO₂ was in the supercritical state.

Methods—Granular xerogel and cryogel scaffold fabrication: Granular xerogel scaffolds (GXS) were made by drying GHS at ambient pressure and room temperature or at 75° C. To convert GHS to granular cryogel scaffolds (GCS), GHS were frozen at −80° C. overnight or snap-frozen in liquid nitrogen, followed by lyophilization in the freeze dryer at ˜0.017 mbar overnight.

Methods—SEM imaging: GHS or bulk hydrogel scaffolds were fabricated in cylindrical acrylic molds (diameter=8 mm and height=3 mm), and converted to GAS, GXS, GCS, or bulk aerogel scaffolds (n=2). To prepare scaffolds for SEM imaging, their top surface was coated with iridium (thickness ˜5 nm) using a low-vacuum sputter coater (Leica EM ACE200, Germany). A scanning electron microscope (Quanta 250 ESEM, Thermo-Scientific, OR, USA) was used at an accelerating voltage of 5 kV and a beam current of 0.67-9.8 nA for S-, M-, and L-GAS or 0.67 nA for GXS, GCS, and bulk aerogel scaffolds to visualize the microscale morphology of samples. For cross-sectional SEM imaging, samples were cut along their sagittal plane, and the exposed cross-sections were coated with an ˜5 nm layer of iridium using a low-vacuum sputter coater (Leica EM ACE200, Germany). A scanning electron microscope (Apreo 2 SEM, Thermo-Scientific, OR, USA) was operated at an accelerating voltage of 5 kV and a beam current of 0.67 nA to visualize the cross-sectional morphology of S-, M-, and L-GAS samples.

Methods—Micro-computed topography (CT): An M-GAS sample (diameter=2 mm and height=1 mm) was scanned using a Zeiss Xradia 620 Versa at the Penn State Center for Quantitative Imaging. Images were acquired with a voxel size of 659.9 nm, a source energy of 70 kV, and a power of 8.5 W. An LE1 filter was applied to reduce potential scanning artifacts. The scan image files were imported to Fiji ImageJ (1.53t, NIH, MD, USA), and exported as an 8-bit TIFF stack for better file handling. Thresholding was applied to perform segmentation and extract the regions of interest. Images were reconstructed using Scout and Scan (version 16.1.14271.44713, Zeiss). Pores were thresholded, separated, and analyzed for connectivity and equivalent radius using DragonFly (version 2022.2.0.1399). Pore network modeling in three-dimensional (3D) images was conducted in Avizo3D (versions 2021.2 and 2023.1.1, ThermoFisher Scientific) to separate pores and pore throats using the Chamfer-Conservative method.

Methods—Swelling test: S-GAS, M-GAS, L-GAS, and bulk aerogel scaffolds, made from their hydrogel counterparts (diameter=12 mm and height=1 mm), were transferred to 50 mm Petri dishes, filled with 8 mL of 37° C. DPBS. Scaffold rehydration was monitored using a Nikon D7500 DSLR camera equipped with AF-S DX NIKKOR 18-140 mm f/3.5-5.6G ED VR lens (Nikon, Japan) for 20 min or 24 h, respectively (n≥3). Screenshots of swollen scaffolds at given timepoints after rehydration were imported to the Fiji ImageJ software (1.53t, NIH, MD, USA), and the scaffold periphery was manually determined using the oval tool in the software. The scaffold equivalent diameter was then calculated from the measured area. The swelling ratio was calculated as the ratio of scaffold diameter at any timepoint after rehydration over the scaffold diameter in the aerogel form. For better visualization, image background was removed using the subject masking feature on Adobe Photoshop Lightroom (version 7.5). A similar test (n=3) was performed using smaller S-, M-, and L-GAS (equivalent diameter ˜2.1-3.0 mm and height ˜1.5 mm).

Methods—Pore characterizations: GAS with varying microparticle sizes were fabricated using cylindrical GHS (diameter=10 mm and height=1 mm). GHS and GAS counterparts were then incubated in DPBS for 24 h at room temperature. To visualize the void spaces among microparticles, ˜10 μL of 15 μM FITC-dextran in DPBS was pipetted onto the GHS or fully rehydrated GAS (rGAS, in DPBS). After ˜10 s, scaffolds were flipped and placed on 50 mm glass-bottom dishes and mounted on a fluorescence microscope (DMi8 THUNDER Imager 3D Cell Culture microscope, Leica Biosystems, Germany) stage. Both two-dimensional (2D) and 3D images of scaffolds were acquired. To generate 3D images, a depth of 259.7, 168.1, or 65.3 μm was used along the Z-axis for GHS and rGAS made of large, medium, or small microparticles, respectively. A MATLAB (MATLAB, version R2023b) code was used to measure the void areas in the 2D images of scaffold layers where hydrogel microparticles had maximum contact, and the diameter of circles with the same areas were determined. The median of equivalent pore diameters was reported for each study group. The void volume fraction (VVF) was measured using built-in software on the microscope (LAS X 5.0.3 Life ScienceMicroscope Software Platform). VVF was determined as the ratio of FITC-dextran-occupied volume divided by the total volume of imaged scaffold section (n=5).

Methods—Compression Analysis: GAS with varying sizes of microparticles were fabricated using cylindrical GHS (diameter=8 mm and height=3 mm). GHS and GAS counterparts were then incubated in DPBS for 24 h at room temperature. An Instron mechanical tester (Instron 5542, MA, USA) was used to conduct compression tests at a 1 mm min⁻¹ compression rate. The excess DPBS around the scaffold was removed using Kimwipes before running the test. Compressive modulus was determined from the slope of the linear regression fitted to the elastic region of compressive stress-strain curve. The elastic region was in the strain range of ˜0.05-0.15 mm mm⁻¹ (n=5). The same experiment was performed on GAS without rehydration to determine the mechanical properties of scaffolds in the dry form (n≥6).

Methods—Rheological analyses: GAS were fabricated using cylindrical GHS (diameter=8 mm and height=1 mm). GHS and GAS counterparts were then incubated in DPBS for 24 h at room temperature. To perform oscillatory rheology, an AR-G2 rheometer (TA instrument, DE, USA) was used. Scaffolds were transferred onto a 25 mm diameter sandblasted bottom plate and were sandwiched by a parallel sandblasted top plate (diameter=8 mm) at 25° C. To eliminate scaffold dehydration during the experiments, a few DPBS droplets were added around the scaffolds. The linear viscoelastic region (LVR) for each scaffold was determined by conducting oscillatory strain sweep tests at a fixed frequency of 1 rad s⁻¹ and strain of 10⁻²-10³%. The frequency range of 10⁻¹ to 10² rad s⁻¹ was selected to perform the frequency sweep tests at 0.1% strain (n=5).

Methods—In vitro cell culture: T-75 cell culture flasks were used to culture NIH/3T3 murine fibroblast cells in supplemented culture media, containing DMEM, FBS (10% v/v), and antibiotics (1% v/v of penicillin-streptomycin, 10,000 U mL⁻¹ penicillin and 10,000 μg mL⁻¹ streptomycin). A cell culture incubator (Eppendorf C170i, Hamburg, Germany) maintained the CO₂ concentration at 5% v/v and temperature at 37° C. The culture media were refreshed every other day, and after ˜80% confluency, cells were subcultured. Cells were counted using an automated cell counter (Countess 2, ThermoFisher Scientific, MA, USA) after being trypsinized by a 0.25% trypsin-EDTA solution, followed by resuspension in supplemented DMEM with a desired cell density for in vitro studies.

Methods—Cell seeding in scaffolds (topical cell seeding and culture in GHS and bulk hydrogel scaffolds): GHS and bulk hydrogel scaffolds were fabricated using cylindrical acrylic molds (diameter=8 mm and height=3 mm). A 24 well non-treated plate was used to incubate scaffolds in DPBS, supplemented with 1% v/v of penicillin-streptomycin at room temperature for 24 h. After discarding DPBS, 20 μL of the cell suspension (5×10⁶ cells mL⁻¹) was added on top of the scaffolds. Samples were then incubated for 30 min to ensure cell adhesion to scaffolds. After 30 min, 1 mL of supplemented culture media was added to each well, containing cell-seeded scaffolds. Samples were then incubated in the cell culture incubator under a 5% v/v CO₂ atmosphere at 37° C.

Methods—Cell seeding in scaffolds (topical cell seeding and culture in GAS and bulk aerogel scaffolds): GAS or bulk aerogel scaffolds were fabricated using cylindrical GHS or bulk hydrogel scaffolds (diameter=8 mm and height=3 mm), respectively, and transferred to a 24 well non-treated plate. The cell suspension (5×10⁵ cells mL⁻¹) with a total volume of 200 μL was added on top of the scaffolds dropwise (˜20 μL each droplet) to rehydrate them, ensuring the whole droplet volume was absorbed. GAS or bulk aerogel scaffolds were then incubated for 30 min at room temperature to ensure cell adhesion to scaffolds. After 30 min, 1 mL of supplemented culture media was added to each well, containing the cell-seeded scaffolds. The scaffolds were then incubated in the cell culture incubator under a 5% v/v CO₂ atmosphere at 37° C. In all in vitro studies, the culture media were refreshed every other day, except for the cell migration assay in which the culture media were not changed.

Methods—Cell seeding in scaffolds (bottom cell seeding and culture in GHS and GAS): GAS were fabricated using cylindrical GHS (diameter=8 mm and height=3 mm). For the bottom cell seeding, 20 μL of the cell suspension (5×10⁶ cells mL⁻¹) was pipetted into each well of a 24 well non-treated plate. Then, GHS or GAS counterparts were placed on top of the cell suspension. To rehydrate GAS, 180 μL of supplemented culture media was added dropwise on top of the scaffolds. After 30 min, each well containing a cell-seeded scaffold was supplied with 1 mL of supplemented culture media, followed by culturing in the cell culture incubator under a 5% v/v CO₂ atmosphere at 37° C.

Methods—Cell viability assessment: To assess the cell viability, live and dead cells were fluorescently stained with two different dyes using the cell viability kit on days 1, 4, and 7. For each scaffold, cells were incubated with 1 mL of a staining solution, containing calcein AM (0.2% v/v), BOBO-3 iodide (0.01% v/v), and Hoechst 34580 (0.1% v/v) in the Live Cell Imaging Solution to stain live cells, dead cells, and cell nuclei, respectively. After incubating the samples in the dark at room temperature for 30 min, the DMi8 THUNDER Imager 3D Cell Culture microscope was used to acquire images. To detect live cells, a channel with ˜488 nm excitation and ˜515 nm emission wavelengths was used, and the excitation and emission wavelengths ˜570 nm and ˜602 nm were used for dead cells, respectively. The channel used for cell nuclei had 392 nm excitation and ˜440 nm emission wavelengths. Cell viability was measured as the fraction of live cell area over the total area of cells (n=3).

Methods—Metabolic activity assessment: PrestoBlue™ HS cell viability kit was used to evaluate the metabolic activity of cells on days 1, 4, and 7 after seeding. A PrestoBlue solution was prepared by diluting the PrestoBlue HS reagent in DMEM at a 1:10 v/v ratio. In each well of 24 well non-treated plates containing a cell-seeded scaffold, 1 mL of the PrestoBlue solution was added, followed by incubation for 3 h at 37° C. in the cell culture incubator under a 5% v/v CO₂ atmosphere at 37° C. The supernatant (100 μL) was then transferred to each well of a 96-well plate, and the fluorescence intensity of solution was measured using a microplate reader (Tecan Infinite M Plex, Mannedorf, Switzerland). The excitation and emission wavelengths were 530 nm and 590 nm, respectively. To correct the measured intensities, the cell-free PrestoBlue solution (PrestoBlue HS reagent in DMEM at a 1:10 v/v ratio) intensity was subtracted from that of cell-containing samples (n=5).

Methods—Cell migration assessment: A CellTracker™ Green (CMFDA) solution in DMSO with a concentration of 0.25% w/v was prepared, followed by dilution in serum-free DMEM at a 1:1000 v/v ratio. The media of confluent cells cultured in T-75 culture flasks were replaced with the dye-containing media, and cells were incubated under a 5% v/v CO₂ atmosphere at 37° C. for 30 min. The dye-containing media were then replaced by 10 mL of supplemented DMEM. Scaffolds (GAS, rGAS, GHS, or bulk hydrogel and aerogel scaffolds) were seeded with cells via the topical or bottom seeding methods explained previously. Using a razor blade, scaffolds were sliced to obtain lateral cross sections on day 0 (˜4 h after cell seeding) and day 3 after seeding. Samples were imaged using the DMi8 THUNDER Imager 3D Cell Culture microscope, and the average cell drawing/migration length was determined using Fiji ImageJ software (1.53t, NIH, MD, USA) (n=5). The in vitro cell drawing/migration assessment using bottom-seeded M-GAS was conducted in two independent experiments under identical conditions, except for the NIH/3T3 passage.

Methods—In vivo subcutaneous implantation of GAS: Animal surgeries were conducted according to an approved protocol by the Institutional Animal Care and Use Committee (#02132, approved on Feb. 24, 2022, and renewed thereafter). To prepare animals for subcutaneous implantation of three distinct study groups (S-, M-, and L-GAS), 16 adult 10-week-old C57BL/6J mice (8 males and 8 females, The Jackson Laboratory, CT, USA) were acclimated for 10 days. To perform the surgery, mice were anesthetized using 2% isoflurane mixed with 5 L min⁻¹ oxygen. Each mouse received an injection of meloxicam (0.5 mL kg⁻¹), followed by preparing the dorsal skin using ethanol (70% v/v) and Chlorhexidine® scrub (2% v/v). Two pockets were formed on the back of each animal by incision without hurting the tissue under the skin (one pocket on the left and one pocket on the right). One scaffold (made from cuboid GHS fabricated in an acrylic mold, length=width=5 mm, and height=3 mm) was implanted in each pocket. Each mouse had two separate groups of GAS. Scaffolds from different study groups were evenly distributed among male and female mice to ensure scaffolds of the same group were not implanted in the same animal and to minimize any sex-specific results. After the surgery, mice were placed in individual cages with ad libitum food and water in a room, equipped with a standard day/night light cycle system, and their weight was monitored every day. Using CO₂ (3 L min¹), mice were euthanized after 7 days of scaffold implantation. Implanted scaffolds were removed from the animals, and the samples were fixed in 4% v/v PFA for 12 h at room temperature. Samples were then maintained in 70% v/v ethanol in an ultrapure water solution for further analysis.

Methods—Histology and immunohistochemistry analyses: Fixed samples were submerged in 70% v/v ethanol, and the ethanol concentration was gradually increased using a tissue processor (Leica TP1020 Automatic Benchtop Tissue Processor, Leica Biosystems, Germany). For this, the samples were initially immersed in 70% v/v ethanol for 30 min, followed by an immersion in 85% v/v ethanol for 45 min, and then 95% v/v ethanol for 40 min, which was repeated twice. Subsequently, they were immersed in 100% v/v ethanol for 40 min. This step was repeated twice. Following the ethanol series, they were immersed in xylene three times, each for 40 min. Finally, they were immersed in paraffin twice, with each immersion lasting 45 min. Paraffin-embedded samples were sectioned at a thickness of 10 μm using a manual microtome (Shandon™ Finesse™ 325 Manual Microtome, Thermo Fisher Scientific, MA, USA). The sections were adhered to Diamond® white glass charged slides.

For hematoxylin and eosin (H&E) staining, sections were loaded on an autostainer (Leica autostainer ST5010 XL, Germany) and underwent deparaffinization at 58° C. for 18 min, followed by three times of 150-s immersion in xylene. Samples were then immersed in 100% v/v and 90% v/v ethanol, for 90 s each, followed by a 1-min immersion in tap water. Sections were then stained via immersion in Hematoxylin 560, Alcoholic Eosin Y 515, Ble Buffer 8, and Define. The process concluded with immersions in 95% v/v and 100% v/v ethanol for 90 s each, followed by a 4-min immersion in xylene.

For immunofluorescence staining, the sections were deparaffinized using the autostainer (Leica autostainer ST5010 XL, Germany) via heating at 58° C. for 18 min, followed by three times of 10-min immersion in xylene. Samples were then immersed in 100% v/v ethanol twice, 3-min each time, followed by 2-min immersion in 95% v/v ethanol and 85% v/v ethanol. The process was concluded with a final 3-min immersion in 70% v/v ethanol. The samples were washed with tap water twice, 4 min each time, followed by immersion in PBS for 5 min.

An antigen retrieval step was then performed using a 10 mM citrate buffer (pH=6) at 60° C. overnight. The tissues were blocked by adding a BSA (1% w/v) and TWEEN® 20 (0.1% v/v) solution in PBS (the blocking solution). A solution containing rabbit anti-mouse recombinant Alexa Fluor© 488 anti-CD31 antibody (1:100) and rabbit anti-mouse recombinant Alexa Fluor© 647 anti-CD68 antibody (1:100) was prepared in the blocking solution. For each tissue section, 150 μL of the solution was added, followed by overnight incubation at 4° C. For CD206 and CD11b staining, a solution was separately prepared by mixing CD206 monoclonal antibody (2A6A10), CoraLite® Plus 488 (1:100) and CD11b Monoclonal Antibody (M1/70), eFluor™ 660, eBioscience™ (1:40) in the blocking solution. Similarly, 150 μL of this solution was added to each section, followed by overnight incubation at 4° C. In parallel, α-SMA monoclonal antibody (1A4), eFluor™ 570, eBioscience™ (1:100) was separately prepared in the blocking solution, and 150 μL was added to each section, followed by incubation at 4° C. overnight. For CD86 staining, a solution of CD86 monoclonal antibody (GL-1) (1:50) was prepared in the blocking solution, and 150 μL was added to each section, followed by overnight incubation at 4° C. An additional step was performed for CD86 staining, where 150 μL of chicken anti-rat IgG (H+L) cross-adsorbed secondary antibody, Alexa Fluor™ 647 (1:200), prepared in the blocking solution, was added to each section and incubated at room temperature for 2 h. All the overnight incubations were carried out using a StainTray™ slide staining system (Thomas Scientific, PA, USA). All volume ratios mentioned in this section are relative to the blocking solution volume. Sections were then rinsed with PBS, followed by mounting using Fluoroshield™ with DAPI (0.0002% v/v DAPI in water). To image the samples, a Leica DMI8 laser scanning confocal microscope, equipped with STELLARIS 5 White Light Laser (Leica Microsystems, Germany) and LAS X Lightning (version 3.5.7.23225) was used. The area of cell nuclei or cells stained using different markers in each sample was measured using Fiji ImageJ software (1.53t, NIH, MD, USA). For the cell nuclei, a region of interest (ROI, width ˜400 μm and length ˜800 μm) was selected, while for cell populations stained with different markers, the ROI was ˜400 μm in both length and width. The fraction of fluorescent area over total ROI area was reported for each cell type and in each sample.

Methods—Statistical analyses: Statistical significance between study groups was assessed using GraphPad Prism (version 9.5.0, MA, USA). Unpaired t-test or the one-way/two-way analysis of variance (ANOVA), followed by the Tukey's post-hoc multiple comparison test were conducted. For all data, the reported averages were based on at least three independent repeats (n≥3) unless otherwise stated. The level of significance is denoted with non-significant (ns) for p≥0.05, *p<0.05, **p<0.01, ***p<0.001, and ****p<0.0001. G*Power software (version 3.1.9.6) was used to determine the number of mice needed for animal surgeries based on a minimum power of 80%, type I error of 0.05, and the effect size of 0.816 (n=8). Samples were evenly distributed among male and female animals to have 8 animals per study group, ensuring scaffolds of the same group were not implanted in each animal. Since some scaffolds were not analyzable, the actual sample size after performing animal surgeries was n=7, 6, and 6 for S-GAS, M-GAS, and L-GAS, respectively. Under this condition, the effect size was 0.825 to retain a minimum power of 80%.

### Results

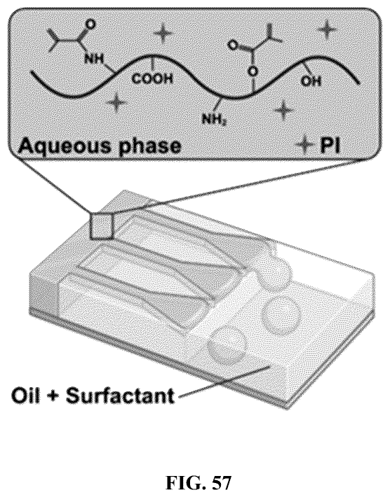

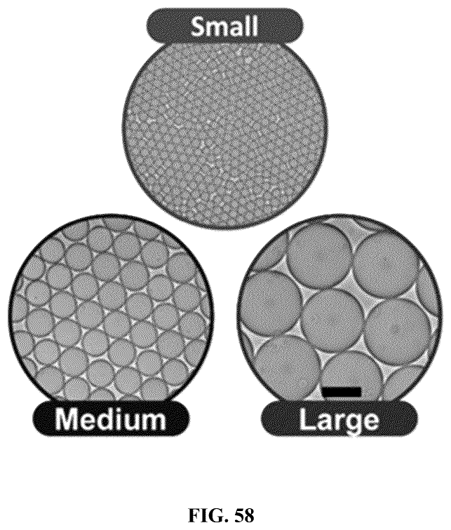

The fabrication process of near-monodispersed GelMA droplets using a high-throughput step-emulsification microfluidic device via forming a water-in-oil emulsion is shown in FIG. 57. FIG. 58 shows the brightfield images of small, medium, and large GelMA droplets, generated using the microfluidic devices with a step size of ˜8, 27, and 60 μm, respectively. These droplets have a narrow size distribution in oil with an average diameter of 27±2, 92±6, and 189±12 μm, respectively. Our hypothesis was that varying sizes of GelMA droplets and consequently hydrogel microparticles formed via crosslinking enable the regulation of GAS pore features, governing cell penetration, adhesion, spreading, and proliferation. GelMA polymer, synthesized using gelatin, a denatured form of collagen (the most abundant protein in the body), benefits from peptide motifs that promote cell adhesion and are susceptible to matrix metalloproteinase (MMP)-mediated degradation.

FIG. 56 shows the process of GelMA GHS fabrication and conversion to GAS. To convert the fabricated droplets into hydrogel microparticles, they are physically crosslinked at 4° C., a temperature at which gelatin partially recuperates the triple-helical structure of collagen. The oil and surfactant are then removed, followed by hydrogel microparticle suspension in a PI solution. After jamming hydrogel microparticles through centrifugation, intra- and interparticle chemical crosslinking via light exposure (wavelength=395-405 nm and intensity=15 mW cm⁻²) yields GelMA GHS. The aqueous phase is then replaced with ethanol to form an alcogel, followed by SCD to yield GAS as the final product. Unlike other drying techniques, such as freeze drying and ambient pressure drying wherein the heterogenous pore size distribution and structural collapse are inevitable, the SCD preserves microparticles and scaffold structural integrity. To preserve the pore structure of hydrogels, SCD is carried out within the single-phase region of solvent/CO₂ phase diagram at a constant temperature, mitigating the disruptive effects of capillary forces at liquid-vapor interfaces within the pores. Therefore, it is imperative that the solvent is miscible with CO₂ at the operating pressure of critical point dryer. This highlights the necessity of a solvent exchange step, wherein water in GHS is replaced with ethanol to form an alcogel. Usually, the operating temperature is set slightly above the critical temperature of pure CO₂. Following solvent extraction at the specified pressure, a gradual isothermal depressurization process yields the desired aerogel product.

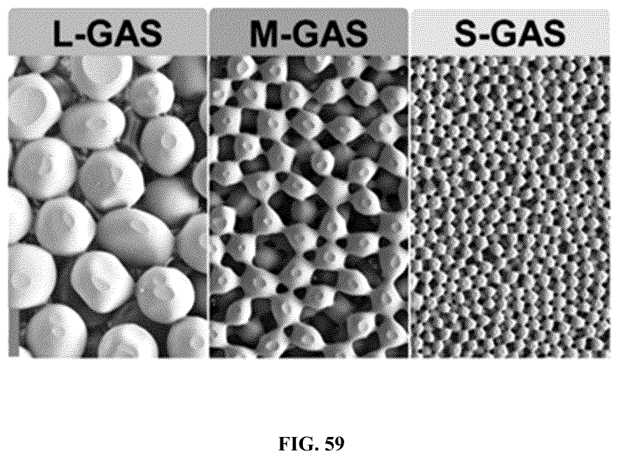

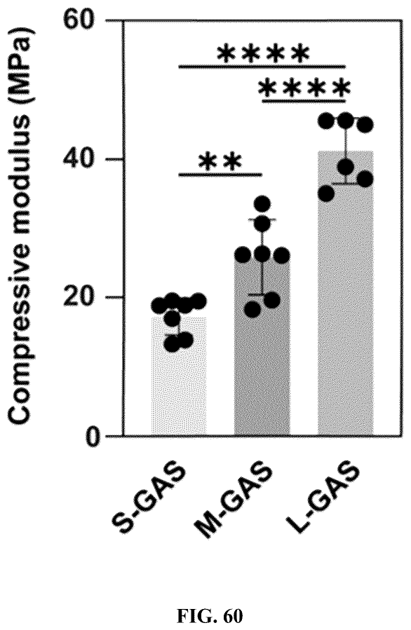

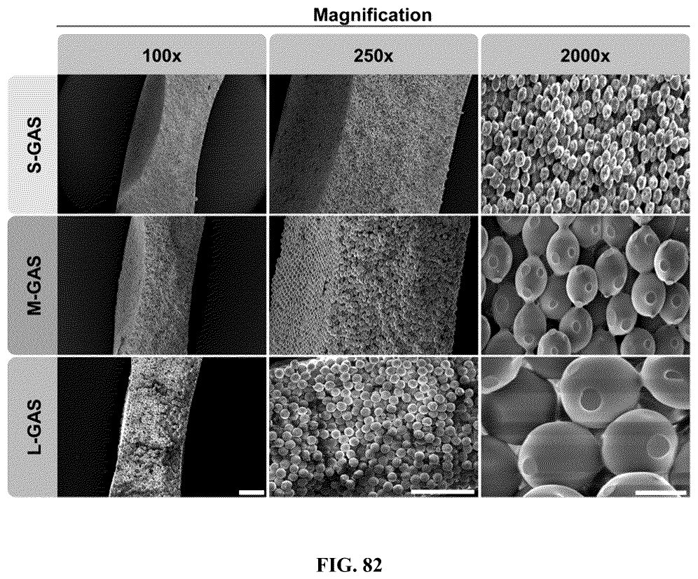

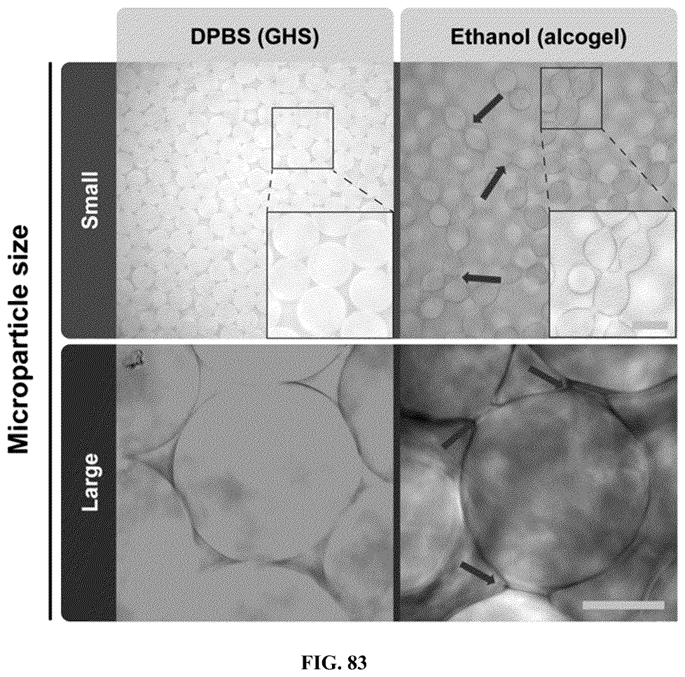

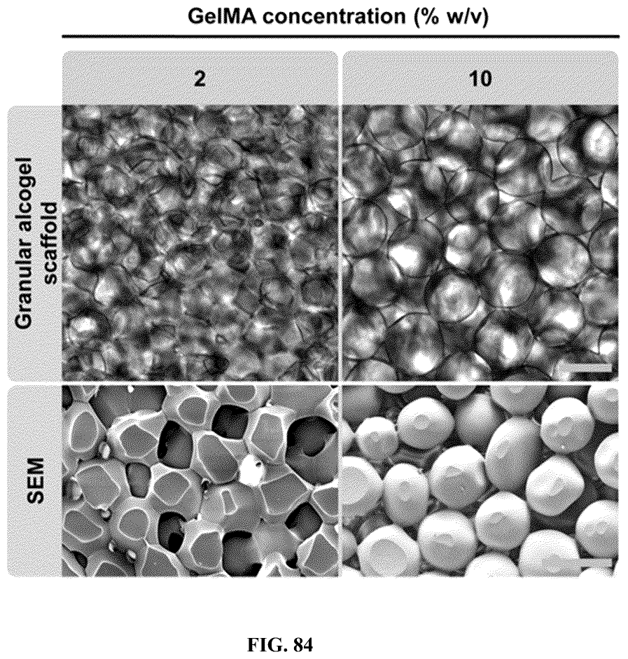

FIG. 59 and FIG. 82 present the SEM images of GAS, fabricated using small, medium, or large microparticles, yielding S-, M-, or L-GAS, respectively. Notably, a morphological change from spherical to elongated building blocks of S- and M-GAS is observed, whereas the microparticles in L-GAS are less deformed. Solvent exchange contributes to the microparticle elongation and neck development, as shown in FIG. 83. Microparticle deformation also depends on the polymer concentration; the lower the GelMA concentration, the more building block deformation for L-GAS is observed (FIG. 84). Unlike GHS, where scaffold compressive modulus increases as the hydrogel microparticles size decreases, the GAS compressive modulus increases by increasing the building block size, as presented in FIG. 60. This may be a result of more substantial microparticle deformation and microparticle-microparticle contact reduction through elongation in smaller microparticles, whereas larger microparticles remain less deformed, experience less strain, and retain their contact during the GHS-to-GAS conversion process. This phenomenon agrees with the reported observations that a smaller interparticle contact in aerogels increases the stress exerted to the necks, reducing aerogel mechanical strength. This explanation is also supported by the Hertz contact theory, where the elastic deformation between two spherical particles leads to a finite contact area that increases with particle size. According to the simplified scaling relations derived from this theory, the contact radius a

$$∝ {( {FR}_{eff} {E}^{* - 1} )}^{\frac{1}{3}}$$

and indentation depth

$$δ ∝ {( {F}^{2} {R}_{eff}^{- 1} {E}^{* - 2} )}^{\frac{1}{3}} ,$$

where F is the normal force between the particles, R_{eff} is the effective radius of the particles (for identical spheres, R_{eff}=0.5R, where R is the particle radius), and E* is the effective modulus accounting for the elastic properties and Poisson's ratios of the particles. Therefore, for the same applied force and material properties, larger particles (higher R) results in a larger contact area. Consequently, the capillary and interfacial forces are distributed over a larger area, reducing local stress at the contact surface. As a result, the extent of localized deformation and neck formation is reduced. In contrast, smaller particles, which develop a smaller contact area, concentrate the force over a smaller region, leading to higher local stresses, more intense local deformation, and consequently, more neck formation between microgels during drying. This is consistent with the SEM images, which show more pronounced neck formation in S-GAS and M-GAS compared with L-GAS. Therefore, the decrease in GAS mechanical strength at smaller building block (microparticle) size implies that microgel deformation during the GHS drying process affects the final mechanical properties of GAS.

FIG. 85 shows the SEM images of GAS, GXS prepared via the ambient pressure drying of GHS at room temperature or 75° C., and GCS fabricated by GHS freezing either at −80° C. or −196° C., followed by lyophilization. Unlike the porous structure of GAS, featuring a preserved structural integrity comparable to GHS, GXS and GCS undergo a post-fabrication structural collapse/disintegration. The capillary forces exerted at liquid-vapor interfaces during the ambient-pressure drying of M-GHS cause a partial structural collapse and the formation of deep cracks in the scaffolds, similar to what has been reported for metal chalcogenide xerogels. The cryosuction phenomenon, where water in a hydrogel is sucked towards the growing ice crystal, causes local dehydration near the ice. The local dehydration during hydrogel freeze drying exerts large shear to microparticles, driving their deformation and scaffold structural integrity loss. This phenomenon is observed and reported in previous studies, where bulk hydrogel scaffolds undergo fracture formation during freeze drying. Besides the lack of structural integrity in GXS, a smaller microparticle-microparticle contact surface (shown with arrows) is observed in GXS fabricated at 75° C. compared with the one fabricated at room temperature. Additionally, microparticles in GCS undergo more extensive deformation when frozen at −196° C. compared with −80° C., which may be attributed to faster cryosuction at lower temperatures, inducing larger shear. Overall, these results imply that drying methods other than SCD, which yields GAS, compromise the scaffold structural integrity and do not enable the precise control of granular scaffold pore features.

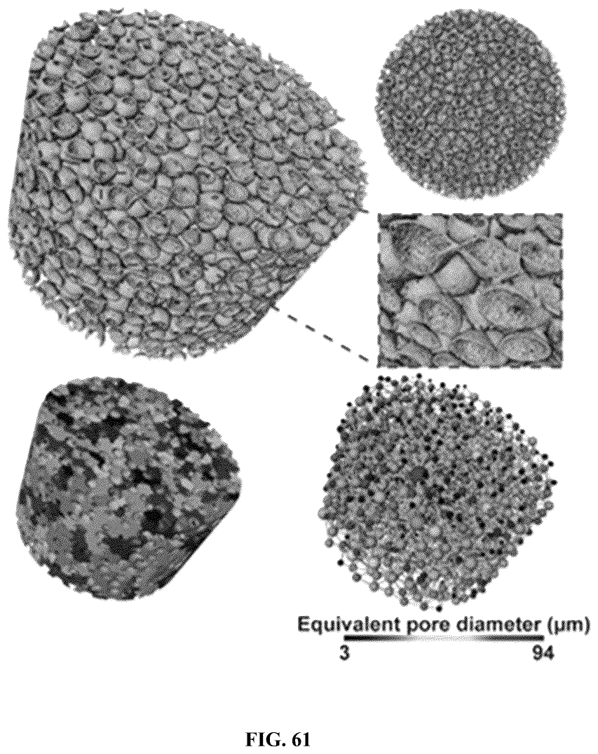

FIG. 61 presents a 3D render of micro-CT scanned M-GAS, showing the preserved scaffold porous structure among covalently bonded aerogel microparticles (spheres). Detected individual pores of M-GAS are visualized in FIG. 61, where varying colors show discrete pores. The average equivalent diameter of detected pores is 43±12 μm, measured using the Chamfer algorithm.

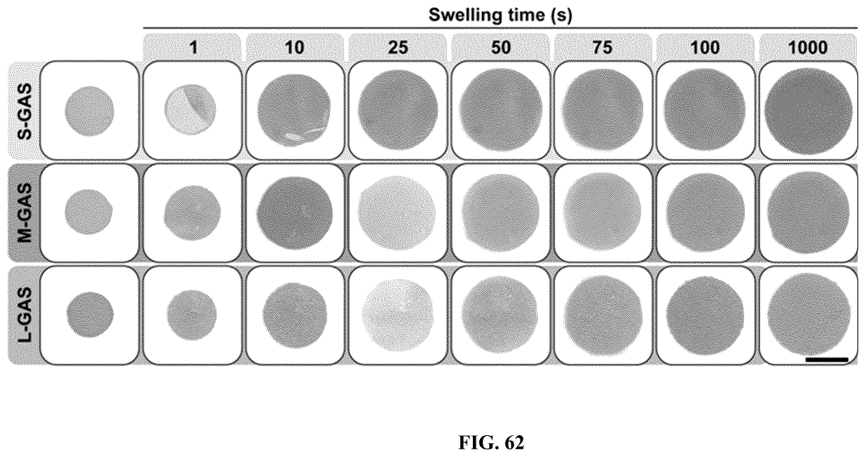

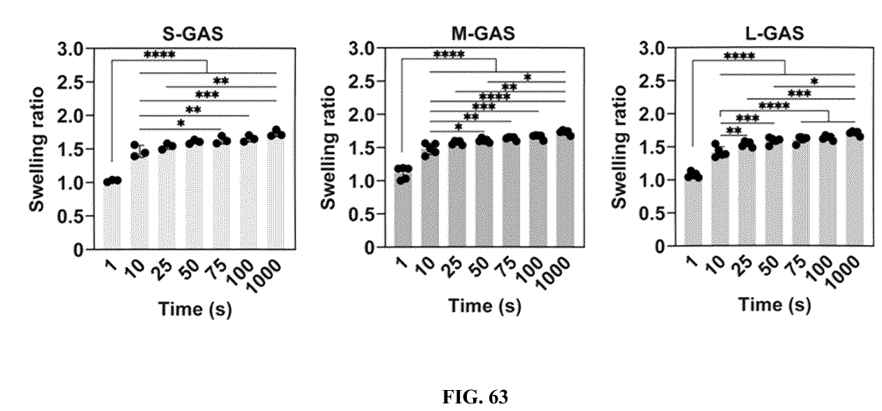

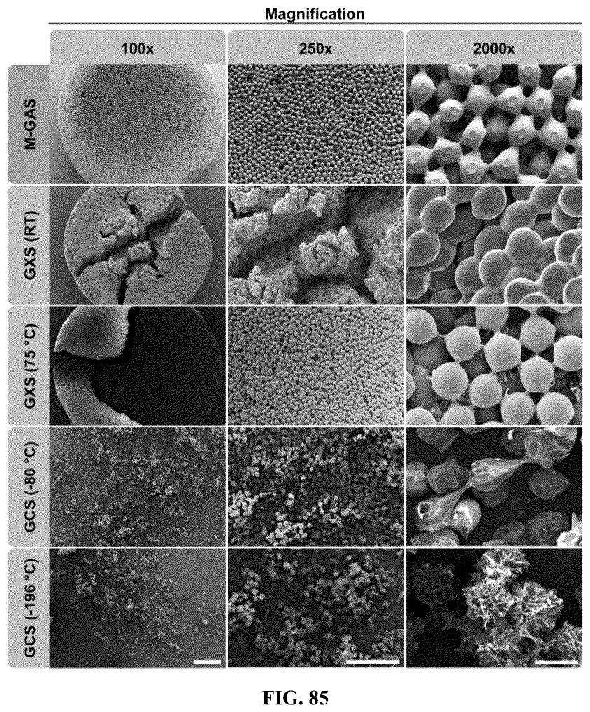

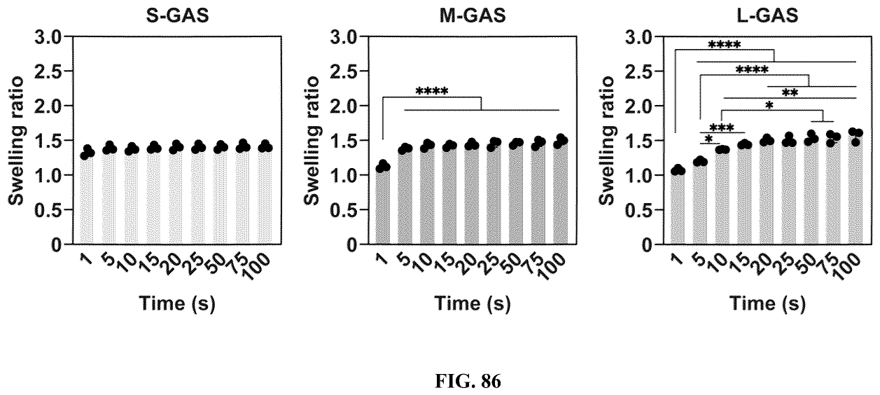

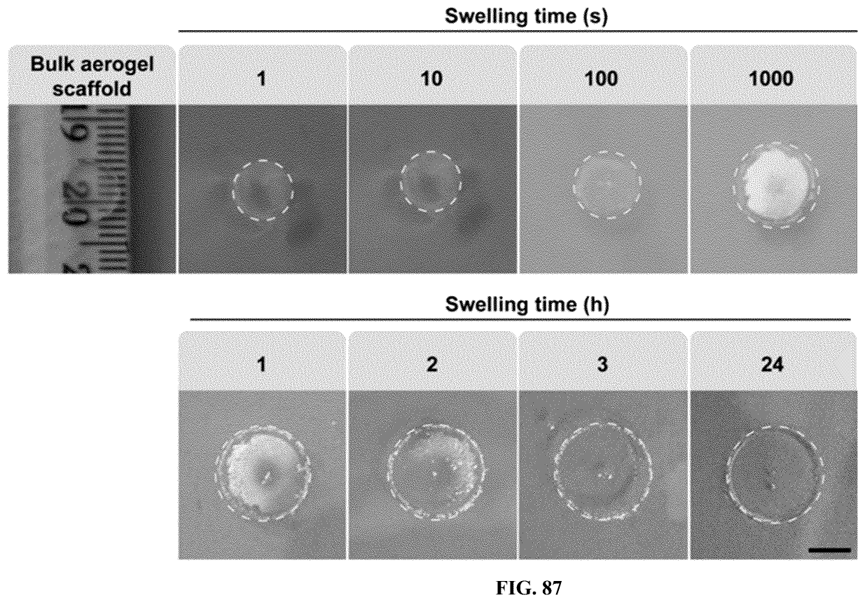

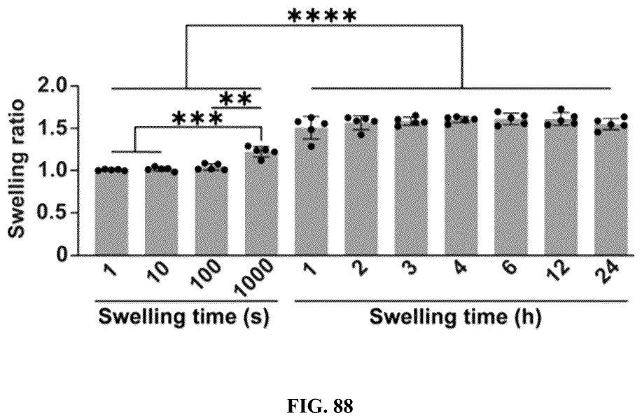

To evaluate the swelling ratio of GAS, fabricated using varying sizes of microparticles, the scaffolds are incubated in DPBS at 37° C. and imaged over time. FIG. 62 shows the images of S-, M-, or L-GAS and their swelling time-lapse after 1, 10, 25, 50, 75, 100, or 1000 s of rehydration. Compared with their initial aerogel state, the scaffold size increases after ˜10 s following contact with DPBS. To quantify the swelling behavior, GAS swelling ratio is calculated based on the ratio of rehydrated scaffold diameter at each time point to the initial GAS diameter. FIG. 63 presents the swelling ratio of S-, M-, and L-GAS at varying time points. S-GAS reach a plateau after ˜25 s, and no further changes in swelling ratio are observed up to 1000 s; however, M- and L-GAS reach a plateau after ˜75 s, suggesting a slower swelling rate compared with S-GAS. S-, M-, or L-GAS reach a swelling ratio of 1.73±0.04, 1.73±0.03, or 1.71±0.03 after 1000 s of swelling, respectively. Another swelling test using smaller GAS (equivalent diameter ˜2.1-3.0 mm and height ˜1.5 mm) shows no significant change in the swelling ratio of S-, M-, or L-GAS after 1, 5, or 15 s, respectively (FIG. 86), suggesting the effect of scaffold size on swelling behavior. Collectively, these findings show that the swelling kinetics of GAS are governed by the microparticle and scaffold sizes. In addition, the swelling behavior of bulk aerogel scaffolds is investigated under the same conditions, as shown in FIGS. 87 and 88. It takes these scaffolds ˜144 times longer than GAS to reach the equilibrium swelling state in DPBS. The prolonged swelling time is attributed to the absence of microscale, interconnected pores in the bulk aerogel scaffolds, which results in significantly slower DPBS transport and absorption rate.

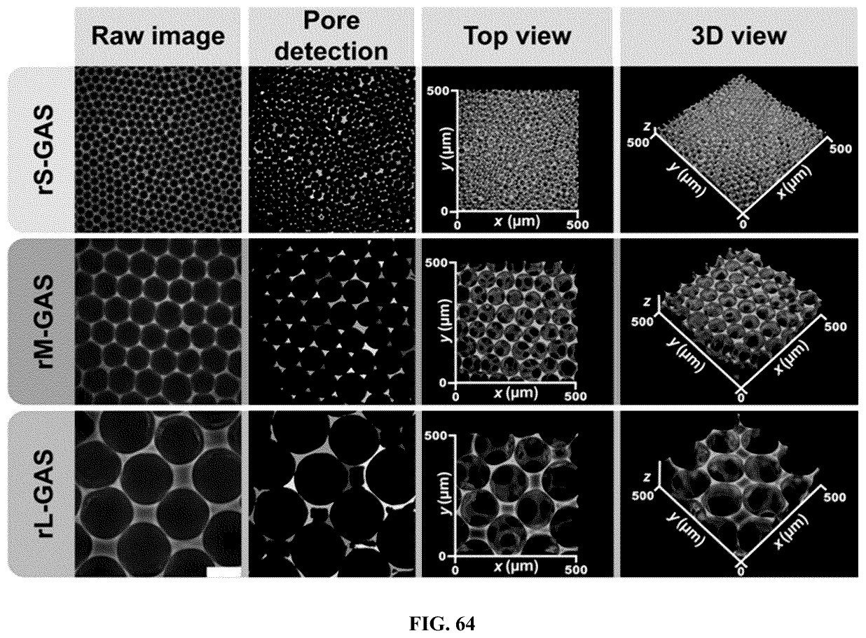

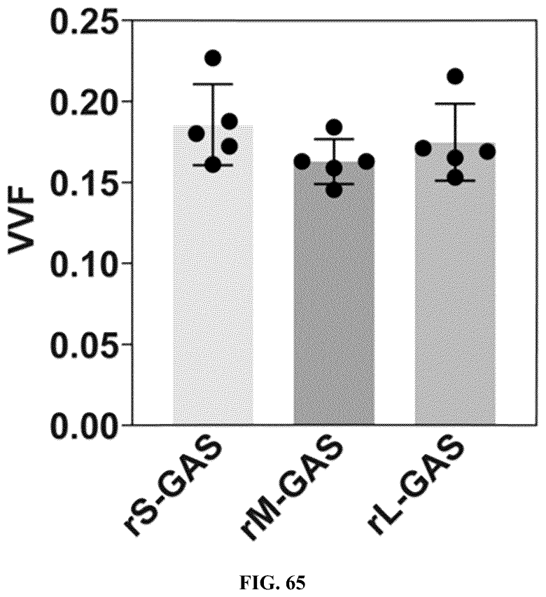

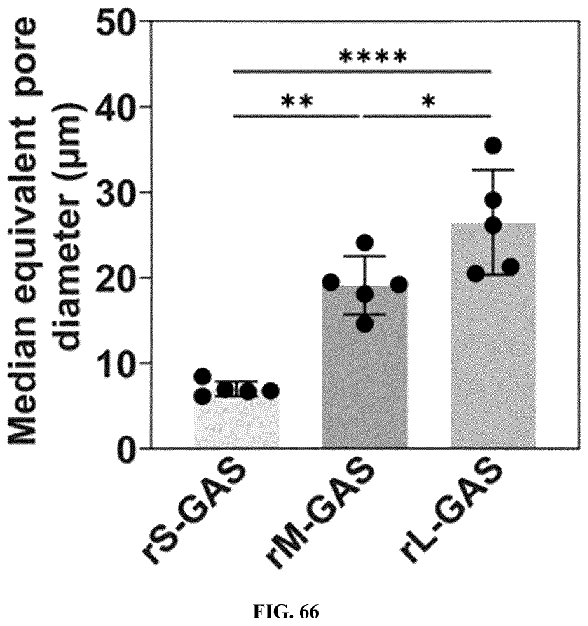

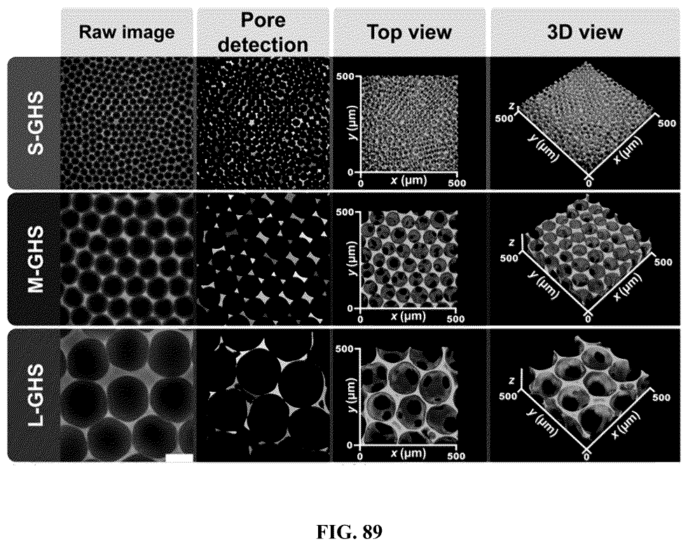

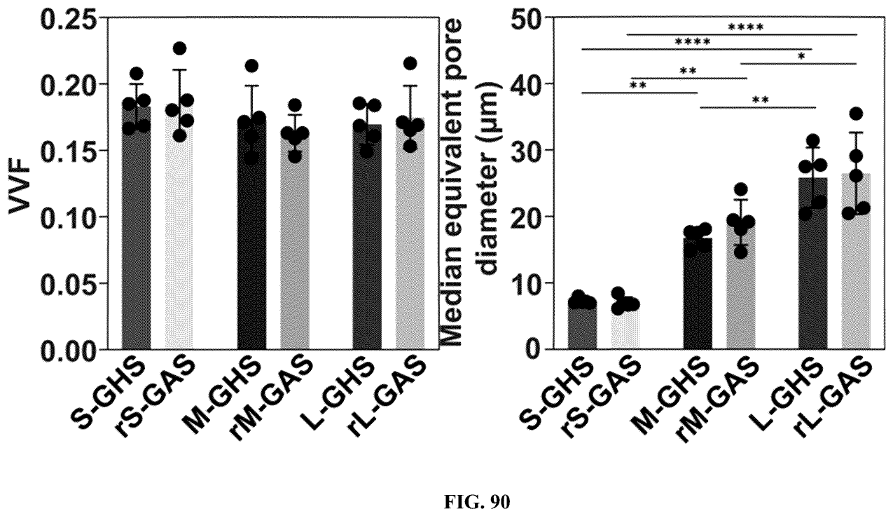

To analyze the pore features of GAS at their equilibrium swelling state, samples are incubated with a high-molecular weight FITC-dextran solution to visualize the scaffold void spaces by fluorescence microscopy. FIGS. 64-66 shows the pore characteristics of rGAS, fabricated using varying microparticle sizes. FIG. 64 shows 2D and 3D fluorescence images of scaffolds, where fluorescence 2D images are used to detect the pores and determine median equivalent pore diameters using a MATLAB code. Top and 3D views of scaffolds show the interconnectivity of pores across all study groups. FIG. 65 presents the VVF of samples, which is calculated by dividing the void volume by the total volume of imaged section. VVF for rS-, rM-, or rL-GAS are 0.19±0.03, 0.16±0.01, or 0.18±0.02, respectively, showing no significant difference among the study groups. FIG. 89 presents the pore characterization of never-dried GHS, prepared using the same hydrogel microparticles sizes used for GAS fabrication. Similar to rGAS, no significant difference in VVF is observed among S-, M-, or L-GHS, which have a VVF of 0.18±0.02, 0.17±0.03, or 0.17±0.02, respectively (FIG. 90). Additionally, GHS and rGAS made of microparticles with the same size do not have significantly different VVF. The VVF reported for both rGAS and GHS in this study is consistent with our previous studies, where the VVF was independent of hydrogel microparticle size in GHS. Pore size is essential for tissue regeneration as it regulates cell infiltration, proliferation, as well as oxygen and nutrient diffusion. The median equivalent pore diameter of rS-, rM-, or rL-GAS is 7±1, 19±3, or 27±6 μm, respectively (FIG. 66). The increase in pore size is associated with the building block size, wherein larger microparticles yield larger pores. Similarly, as shown in FIG. 90, the median equivalent pore diameter in S-, M-, or L-GHS are 7±1, 17±1, or 26±5 μm, respectively. This increasing trend in pore size is in agreement with previous GHS studies in which larger hydrogel microparticles lead to larger pore sizes. Importantly, no significant difference in the median pore diameter is observed between rGAS and their GHS counterparts, showing that GAS at their equilibrium swelling state have pore features comparable to the GHS, fabricated using a similar microparticle. This proves that SCD preserves pore characteristics of granular scaffolds, including median equivalent pore diameter and VVF.

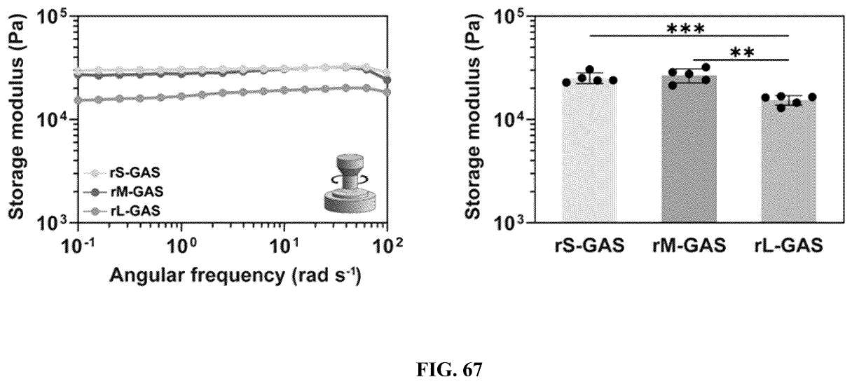

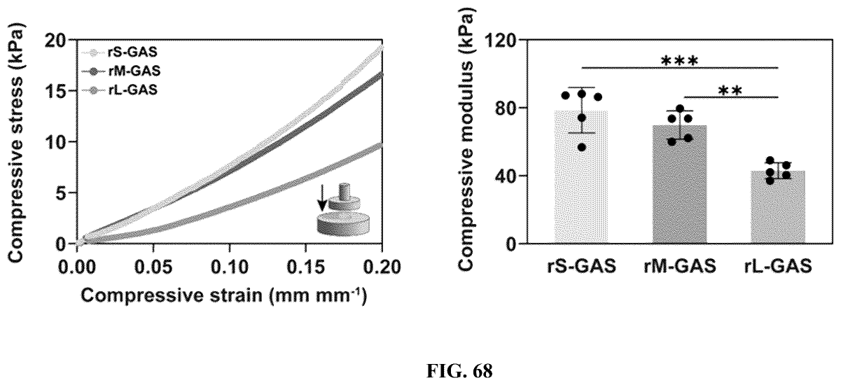

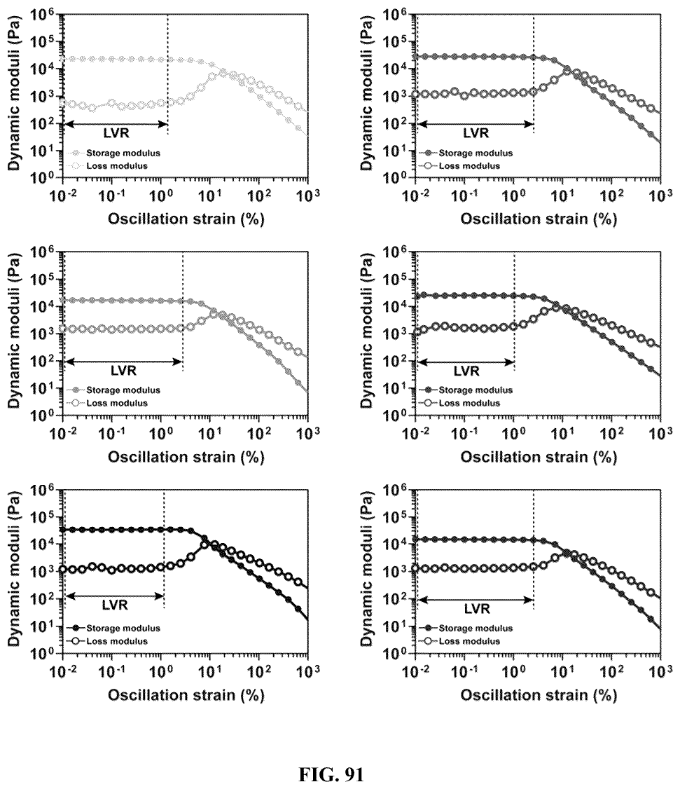

To determine the mechanical and rheological properties of rGAS, compression and oscillatory frequency sweep tests are performed (FIGS. 67 and 68). The scaffold storage modulus versus angular frequency at constant shear strain (0.1%) is presented in FIG. 67 (also see the LVR in FIG. 91). At any given angular frequency between 101 and 101 rad s-, rS- and rM-GAS storage modulus is higher than that of rL-GAS. FIG. 68 shows that the average storage modulus of rS-, rM-, and rL-GAS is 25±3, 27±4, and 15±2 kPa, respectively. These results show no significant statistical difference between the average storage modulus of rS- and rM-GAS, while both are significantly higher than rL-GAS, which is attributed to the smaller contact area per unit volume among adjacent rehydrated microparticles in the rL-GAS.

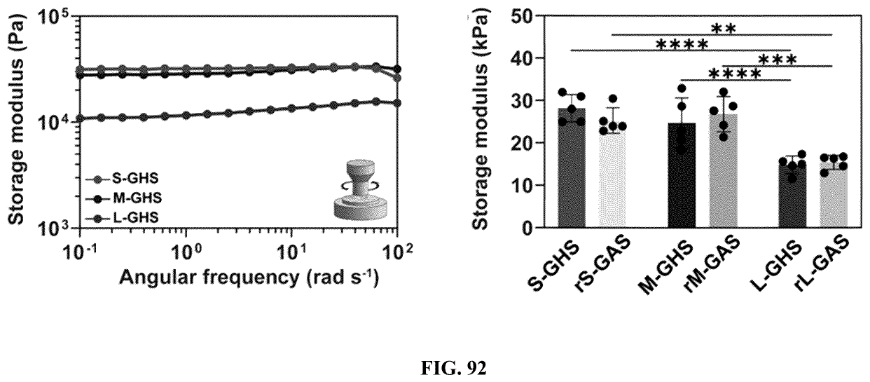

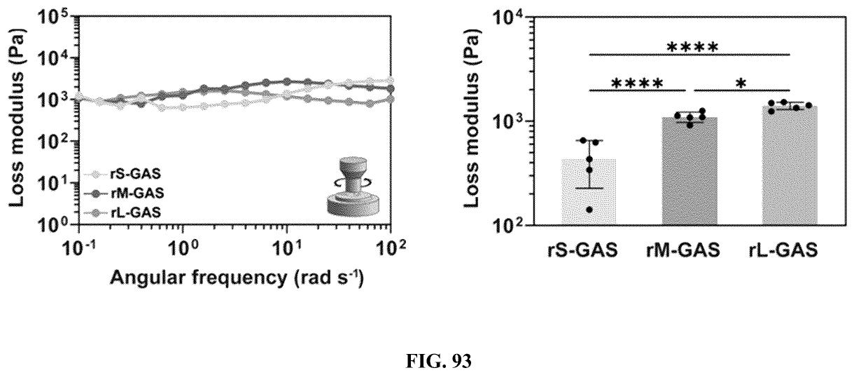

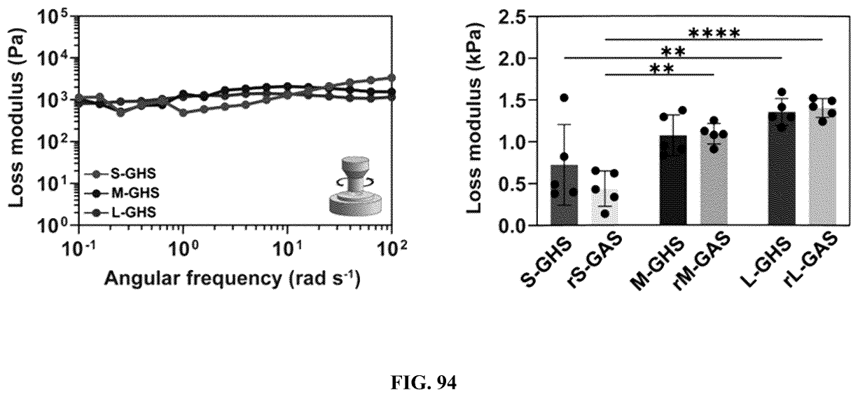

FIG. 92 presents the storage modulus of S-, M-, and L-GHS versus angular frequency at constant shear strain (0.1%). At any given angular frequency between 101 and 101 rad s-, S- and M-GHS storage modulus is higher than that of L-GHS. FIG. 92 shows the average storage modulus of rGAS and GHS fabricated using varying microparticle sizes. The average storage modulus of scaffolds fabricated using large microparticles is lower than that of scaffolds made up of small or medium microparticles. Additionally, the average storage modulus of rGAS has a similar trend to that of GHS, with no significant difference observed between rGAS and their GHS counterparts. FIG. 93 presents the loss modulus versus angular frequency, ranging from 10⁻¹ to 10² rad s⁻¹ at constant shear strain (0.1%). At an angular frequency of 1 rad s⁻¹ and shear strain of 0.1%, the storage modulus of rGAS is at least an order of magnitude greater than its loss modulus. Additionally, FIG. 93 shows that the average loss modulus of rS-, rM-, and rL-GAS is 0.4±0.2, 1.1±0.1, and 1.4±0.1 kPa, respectively, indicating an increasing trend by increasing microparticle size, with a significant statistical difference between the study groups. Furthermore, the average loss modulus in all study groups is at least one order of magnitude lower than the storage modulus, which is common in gels. The GHS loss modulus versus angular frequency at constant shear strain (0.1%) is presented in FIG. 94. Similar to the rGAS, fabricated using varying microparticle sizes, at an angular frequency of 1 rad s- and shear strain of 0.1%, the storage modulus of GHS is at least an order of magnitude greater than its loss modulus. FIG. 94 shows the average loss modulus of rGAS and GHS, fabricated using varying microparticle sizes. The average loss modulus of scaffolds shows an increasing trend by increasing the microparticle size with no significant difference between rGAS and their GHS counterparts.

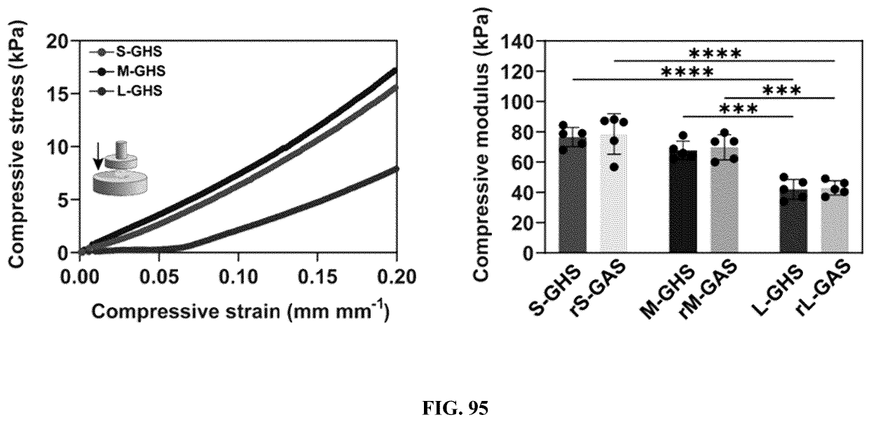

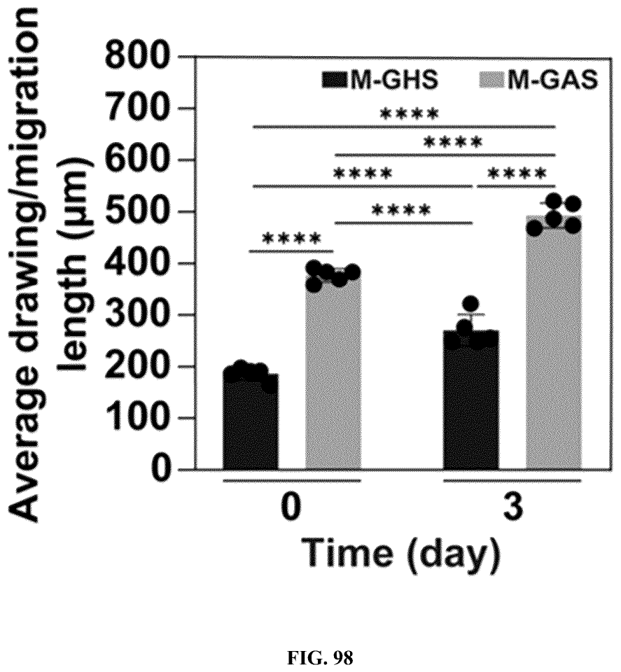

FIG. 68 presents the compressive stress-strain curves of rGAS. The slope of best linear fits to these curves in the range of ˜0.05-0.15 mm mm⁻¹ (elastic region) is reported as the scaffold compressive modulus. The average compressive modulus of rS-, rM-, and rL-GAS is 79±13, 70±8, and 43±5 kPa, respectively (FIG. 68). The average compressive modulus decreases as the microparticle size increases; however, the difference between rS-GAS and rM-GAS is not statistically significant. This trend, which is also observed in the storage modulus of rGAS, may be attributed to the higher contact area per unit volume among rehydrated smaller microparticles. FIG. 95 presents the compressive stress-strain curves of GHS. The slope of best linear fits to these curves in the range of ˜0.05-0.15 mm mm⁻¹ (elastic region) is reported as the scaffold compressive modulus. FIG. 98 shows the average compressive modulus of rGAS and GHS fabricated using varying microparticle sizes. The average compressive modulus of scaffolds decreases by increasing the microparticle size, and there is no significant difference between rGAS and their GHS counterparts. Overall, the mechanical and rheological properties of rGAS and their GHS counterparts are comparable, which may mimic the mechanics of human soft tissues.

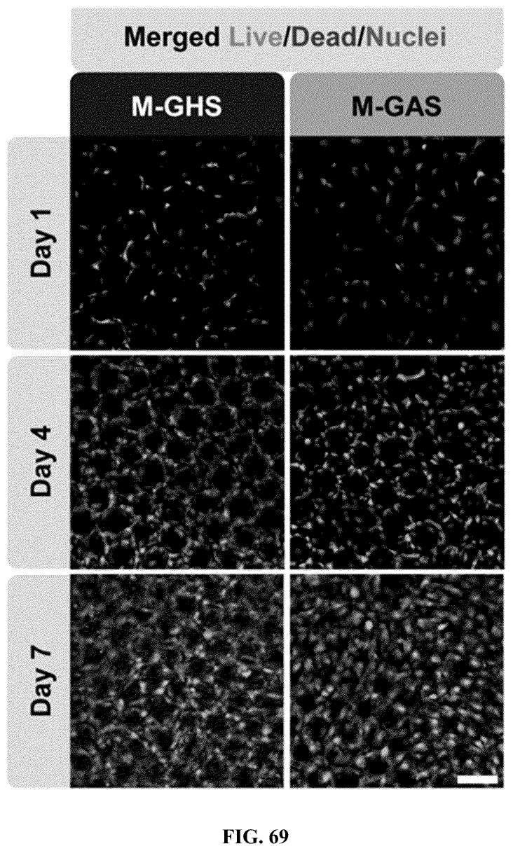

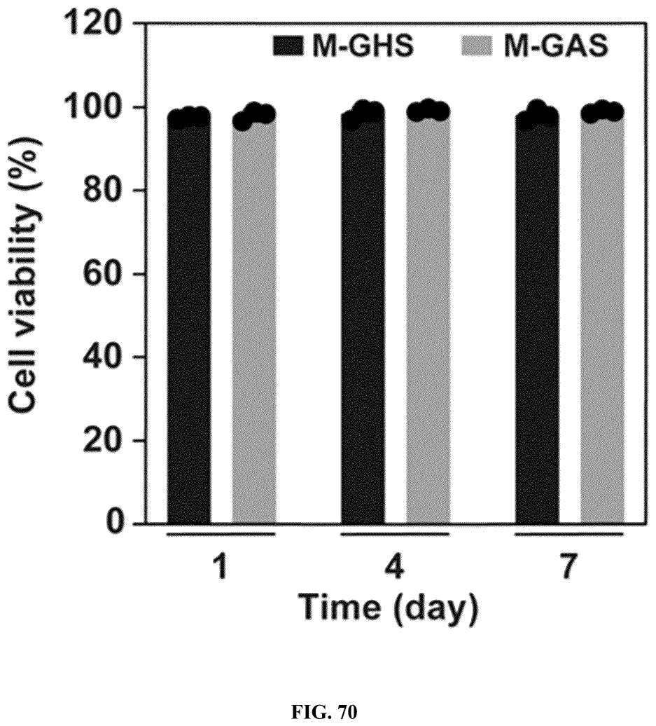

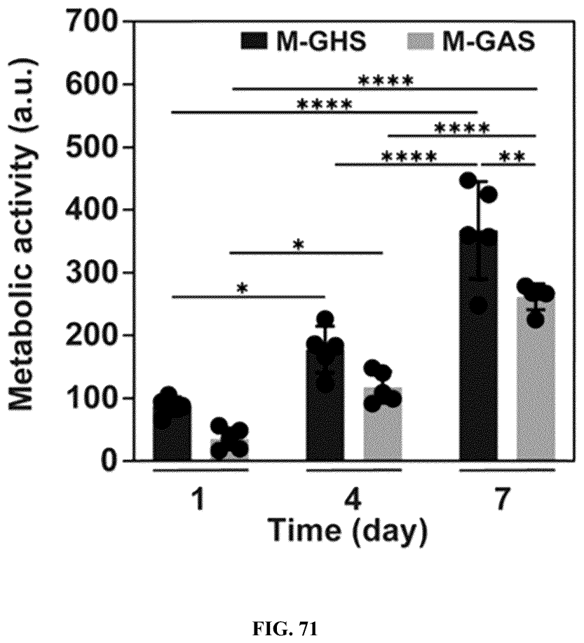

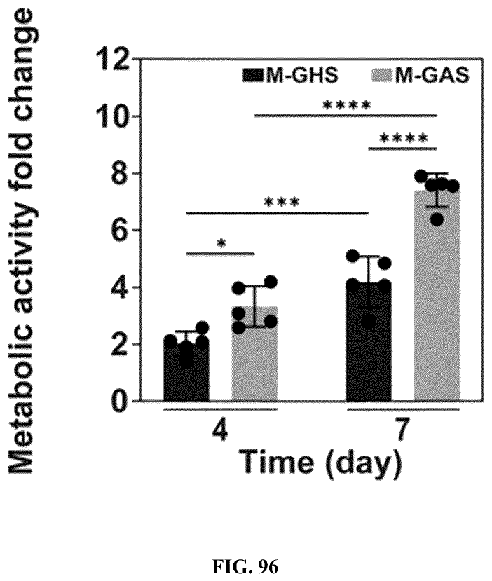

In vitro analyses are conducted on M-GAS and its never-dried GHS counterpart to evaluate NIH/3T3 murine fibroblast cell viability, metabolic activity, and average drawing/migration length within the scaffolds. FIG. 69 shows the live/dead/Hoechst staining of cells in M-GHS and M-GAS, imaged over one week. Live cells, dead cells, or cell nuclei are shown, qualitatively implying a similar cell distribution in the GAS and GHS at each timepoint. Live/dead images are used to quantify the cell viability in the scaffolds over time by normalizing the area of live cells to the total cell area (live and dead), as shown in FIG. 70. Approximately 98% of cells remain viable in M-GAS or M-GHS on days 1, 4, and 7 after seeding, and no significant difference in cell viability is observed among the study groups, which indicate the biocompatibility of GAS in vitro. FIG. 71 presents the PrestoBlue assay outcome, showing that the cell metabolic activity in the scaffolds monotonically increases over time. The metabolic activity in M-GHS is higher than that in M-GAS after 7 days of topical seeding, which may be a result of large capillary forces exerted on cells drawn into M-GAS, reducing their metabolic activity on day 1. FIG. 96 shows the cell metabolic activity fold change on days 4 and 7 with respect to the average of metabolic activity on day 1. The increase in metabolic activity for M-GAS is ˜3.3- and ˜7.4 fold on days 4 and 7, respectively, and the metabolic activity increase of M-GHS is ˜2.0- and ˜4.2 fold on days 4 and 7, respectively, which are significantly lower than that for M-GAS. This may be because of the cell crowding at the surface of M-GHS causing partial over confluency, whereas cells in M-GAS occupy a larger scaffold volume.

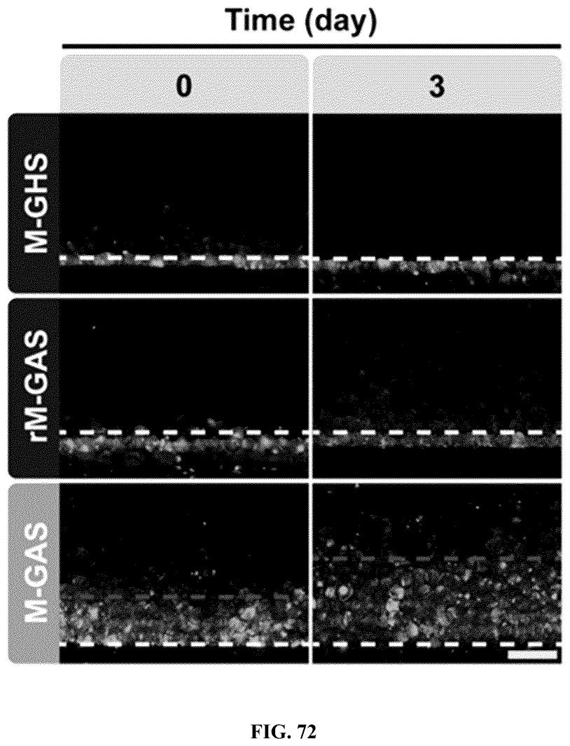

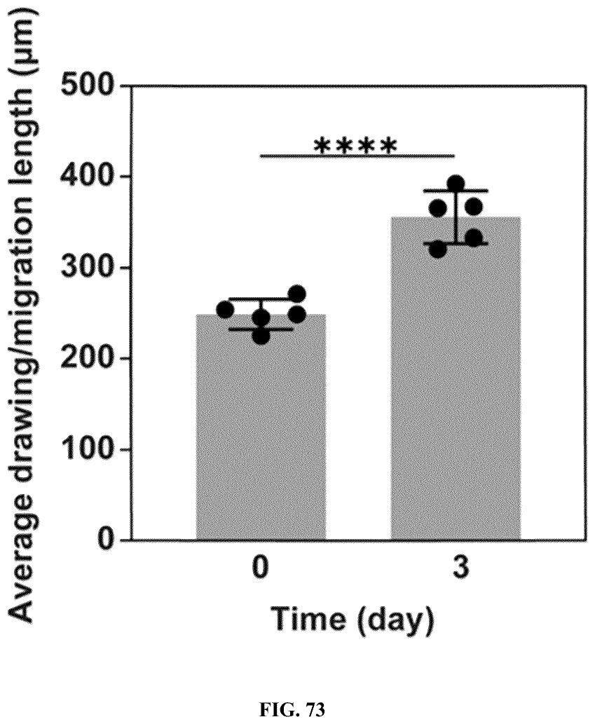

To evaluate the average cell drawing/migration length in the scaffolds, M-GHS, rM-GAS, and M-GAS are bottom seeded with the fibroblast cells. FIG. 72 shows the cross section of scaffolds, imaged on day 0 (˜4 h after cell seeding) and day 3. Unlike M-GAS, no significant cell drawing or migration is observed in M-GHS and rM-GAS after 3 days. The average cell drawing length in M-GAS on day 0 is ˜249±8 μm, and cells migrate up to ˜356±13 μm deep into the scaffold on day 3, as presented in FIG. 73. This may be attributed to large capillary forces at the solid-liquid interface during rapid cell suspension absorption, drawing the cells into M-GAS. A similar phenomenon has been reported for calcium phosphate-based bone scaffolds in which cells are drawn into the scaffolds via capillary forces. The capillary forces may deform cells while squeezing them through the interconnected microscale pores of dry scaffolds.

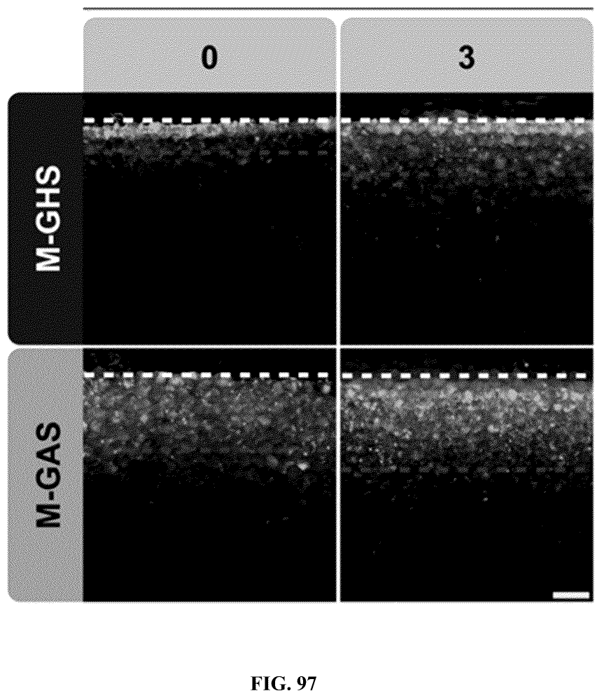

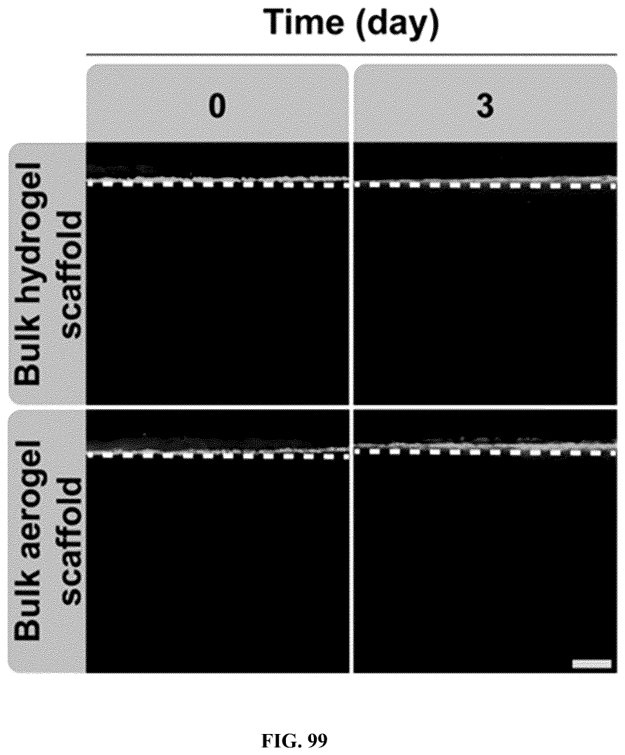

The average cell drawing/migration length following topical seeding in GelMA M-GAS, M-GHS, and bulk hydrogel and aerogel scaffold counterparts is presented in FIGS. 97-99. On day 0 (˜4 h after cell seeding), the average cell drawing length in M-GAS and M-GHS is 377±13 μm and 187±12 μm, respectively. On day 3, cells migrate up to 493±24 μm and 271±31 μm deep into M-GAS and M-GHS, respectively. At both time points, the cell drawing/migration length is significantly higher in M-GAS than in M-GHS. As pointed out earlier, we attribute this difference to stronger capillary forces in M-GAS, which result in higher cell suspension absorption. It is important to note that the change in cell migration on day 3 compared with the average cell drawing length on day 0, is not statistically significant between M-GHS and M-GAS, possibly as a result of similar pore features. On the other hand, cells remain on the surface of GelMA bulk hydrogel and aerogel scaffolds because the scaffold pore size is significantly smaller than the cell size, which is unsuitable for cell drawing/migration.

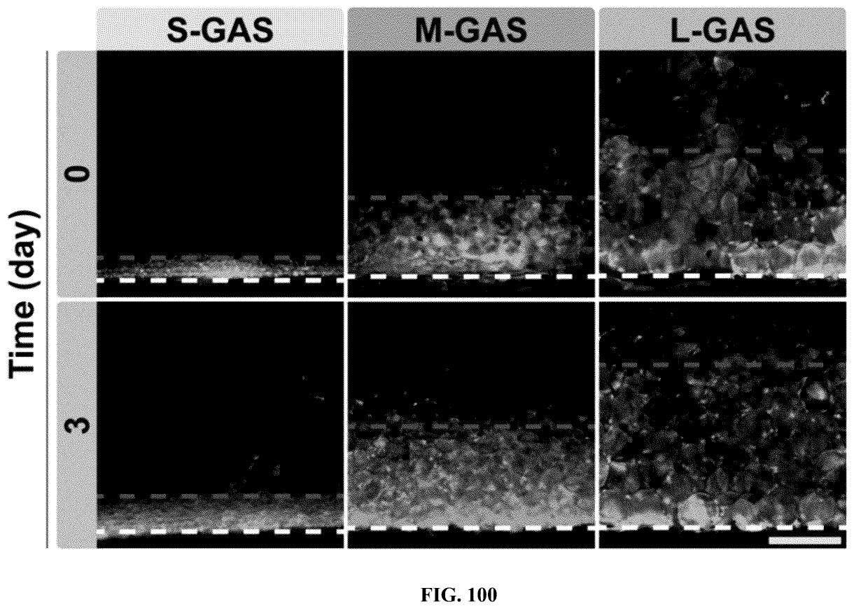

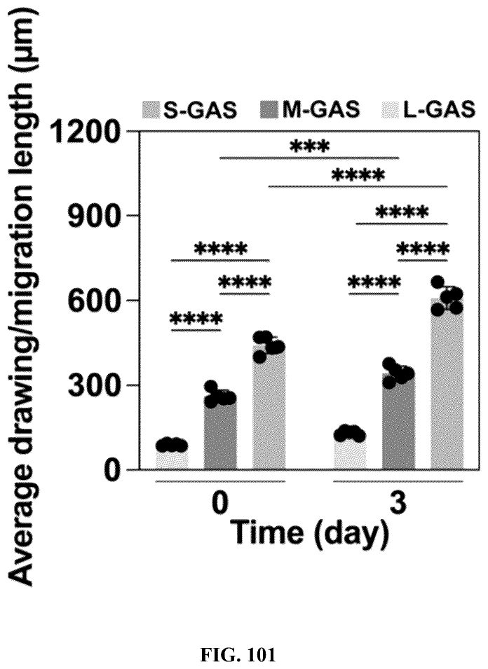

To evaluate the effect of pore size on average cell drawing/migration length in the scaffolds, S-, M-, and L-GAS are bottom seeded with the fibroblast cells. FIG. 100 presents the cross-sectional fluorescence microscopy images of samples, acquired on day 0 (˜4 h after cell seeding) and day 3. The results show that an increase in pore size corresponds to a greater cell drawing/migration length in GAS. The average cell drawing length in S-, M-, and L-GAS on day 0 is 88±3, 261±21, and 442±29 μm, respectively. By day 3, cells migrate up to 129±8, 342±26, and 609±40 μm into the S-, M-, and L-GAS, respectively, as presented in FIG. 101. Although smaller pore sizes may enhance initial capillary-driven cell drawing, our results show that cell transport is also governed by the accessibility of void spaces. In the scaffolds made up of smaller particles, smaller pore sizes and narrower pore throats may impose greater physical confinement, limiting the extent of cell drawing. In contrast, the scaffolds comprising larger microparticles have larger pores and wider pore throats, resulting in higher cell drawing/infiltration.

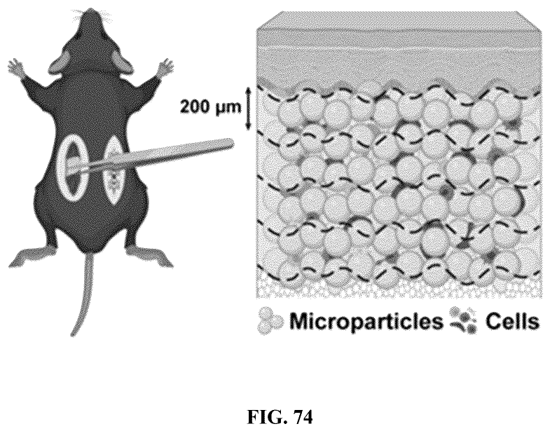

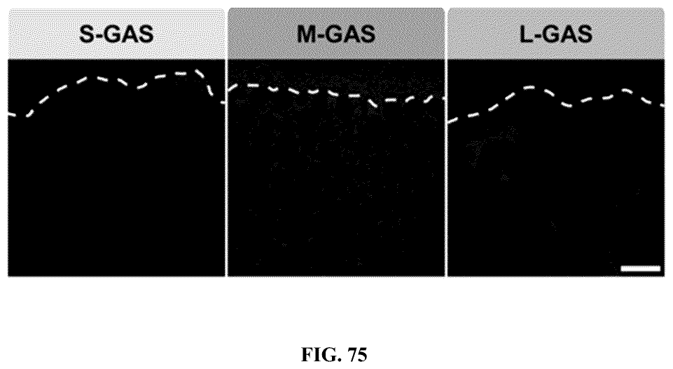

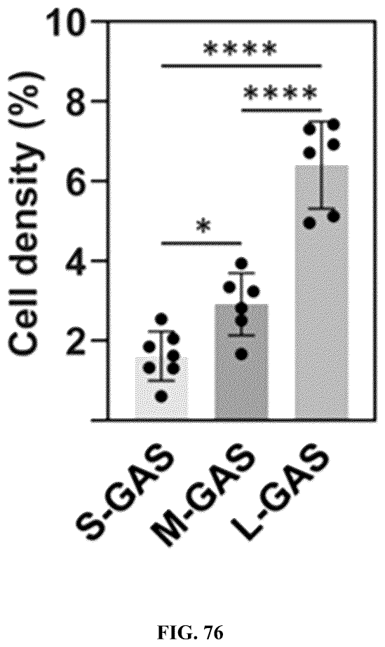

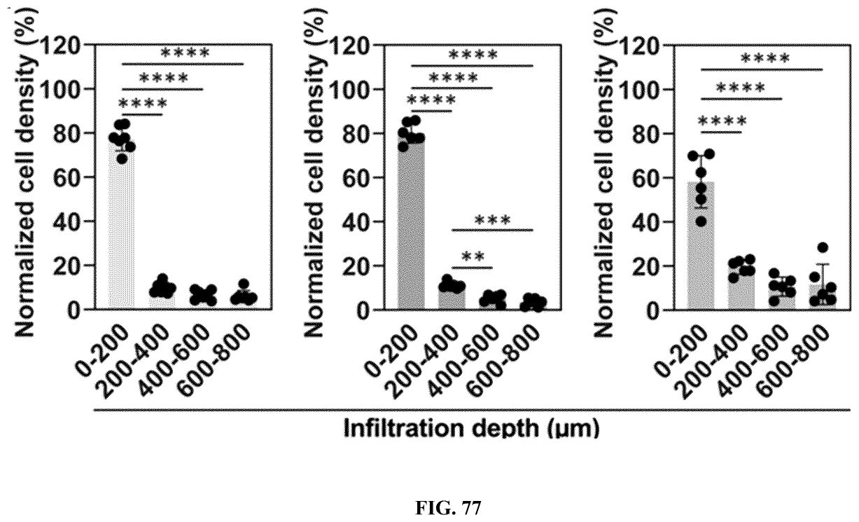

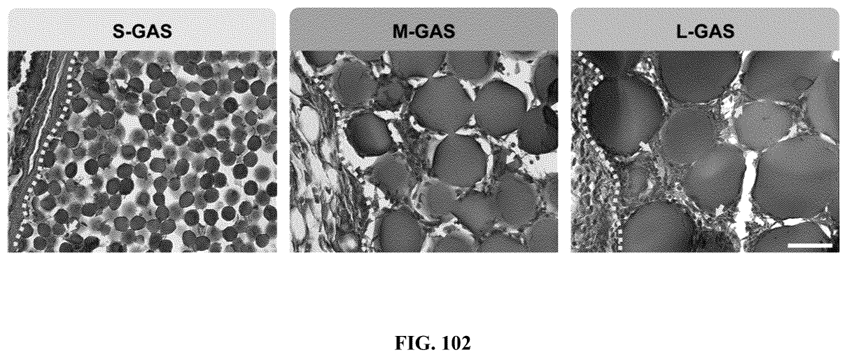

To evaluate the effect of microparticle size on endogenous cell recruitment in acellular S-, M-, and L-GAS, scaffolds are subcutaneously implanted in mice. FIGS. 74-79 presents the results of cell infiltration in the GAS, fabricated using microparticles of varying sizes after 7 days of implantation. A schematic of GAS subcutaneous implantation into the pockets incised on the mouse dorsal skin is shown in FIG. 74. FIG. 75 shows stained cell nuclei, where a higher cell density in L-GAS is observed qualitatively compared with S- and M-GAS. FIG. 102 shows H&E-stained scaffolds. The results confirm cellular infiltration into the scaffolds, with a greater number of infiltrated cells and increased ECM deposition observed in M- and L-GAS compared with S-GAS. FIG. 76 presents the normalized cell density in the GAS, calculated by dividing cell nucleus area by the total ROI area (length=800 μm and width=400 μm). The normalized cell density for S-, M-, or L-GAS is 1.6±0.2, 2.9±0.8, or 6.4±1.1%. Notably, the larger the microparticles, the higher the normalized cell density in implanted samples, which may be a result of larger void spaces facilitating cell infiltration among the microparticles. FIG. 77 show the cell density distribution in 200 μm-intervals starting from the tissue-scaffold interface, normalized to the total ROI area. In the first interval, the normalized cell density in S-, M-, and L-GAS is 77±6, 80±5, and 58±12%, respectively, which show that most of the cells are located within the first 200 μm of tissue-scaffold interface. While a decrease in cell infiltration is observed in the following intervals, cell infiltration remains uninhibited.

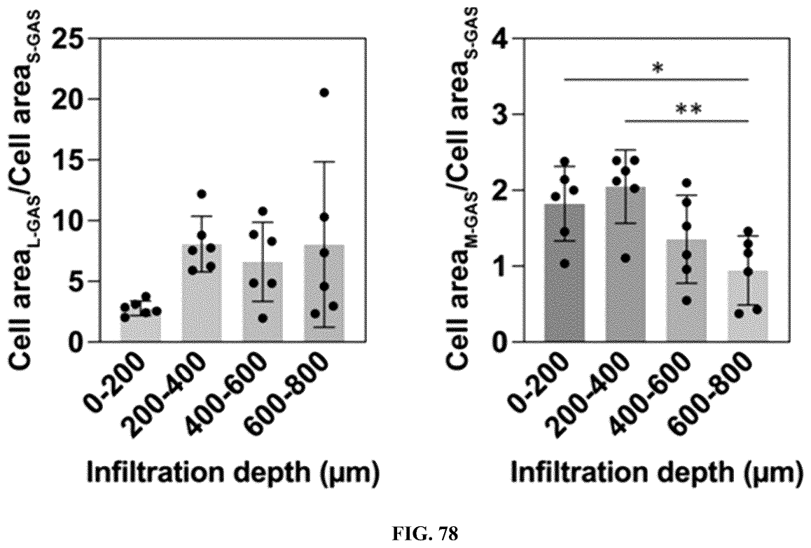

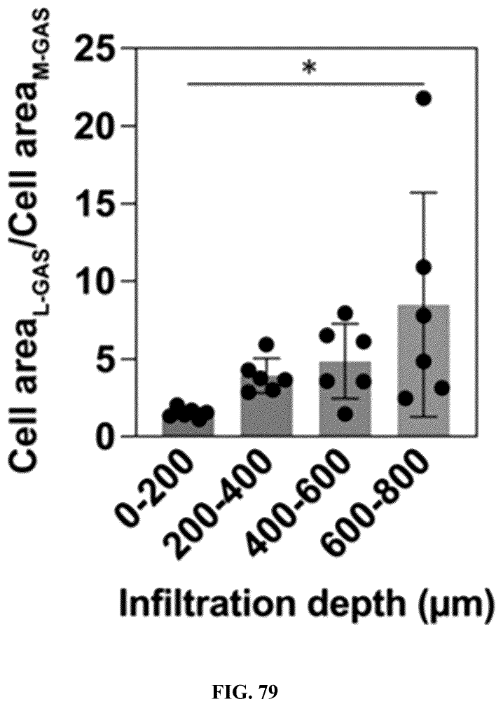

To evaluate cell infiltration across different layers of implanted GAS, the cell nucleus area at each layer (depth=200 μm) in L- and M-GAS is normalized to the average cell area in the same layer of S-GAS, as shown in FIG. 78. In the first layer, L-GAS undergoes ˜2.8 times higher cell infiltration compared with the same layer in S-GAS. This ratio further increases up to ˜8 times in the deepest scaffold layer (depth=600-800 μm). In M-GAS, while cell infiltration in the first layer is ˜1.8 times higher than that in S-GAS, the ratio drops to ˜1 in the deepest layer (600-800 μm). Furthermore, to compare cell infiltration between L-GAS and M-GAS, the cell nucleus area at each layer of L-GAS is normalized to the average cell area in the same layer of M-GAS, as shown in FIG. 79. The first layer of L-GAS undergoes ˜1.5 times increase in cell infiltration compared with the same layer of M-GAS, and the ratio increases up to ˜8.5 in the deepest layer (600-800 μm). The increased infiltration, particularly in the deeper regions of scaffolds comprising larger microparticles, may be attributed to the larger pore sizes, facilitating cell infiltration and tissue integration.

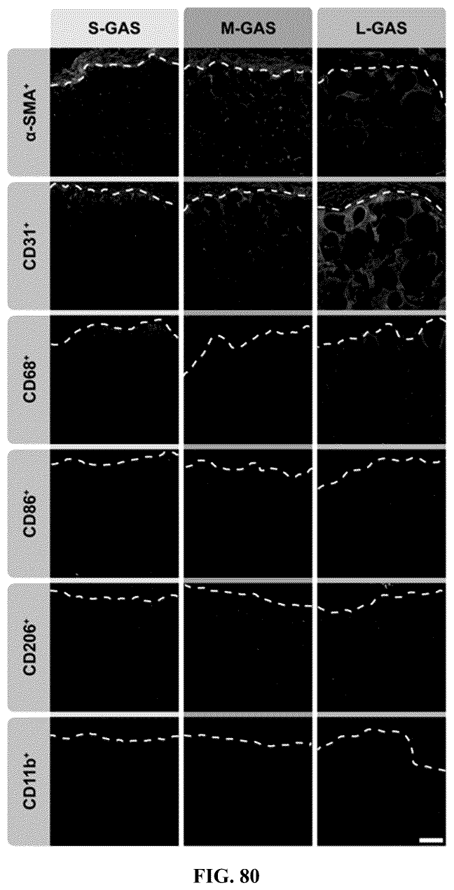

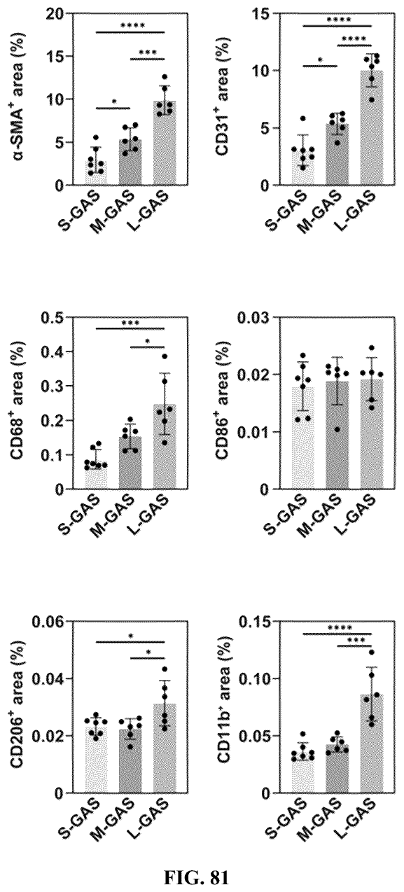

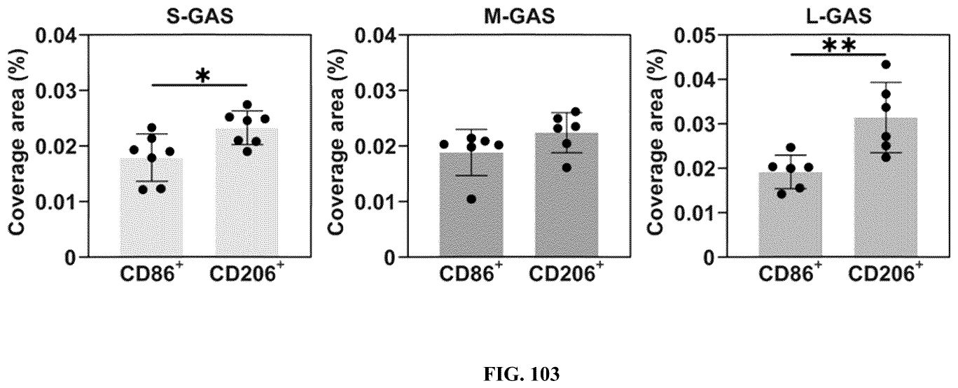

Further characterizations of implanted GAS are conducted by staining the cells in the scaffolds for markers, including α-SMA to identify myofibroblasts (α-SMA⁺), CD31 for endothelial cells (CD31⁺), CD68 for macrophages (CD68⁺), CD86 for M1 macrophages (CD86⁺), CD206 for M2 macrophages (CD206⁺), and CD11b for leukocytes (CD11b⁺). FIGS. 80-81 presents immunofluorescence images of cells infiltrated into the scaffolds, along with the quantification of each cell population after 7 days of implantation. FIG. 80 shows the immunofluorescence images for α-SMA⁺, CD31⁺, CD68⁺, CD86⁺, CD206⁺, and CD11b⁺ cells in S-, M-, and L-GAS, where a higher number of cells expressing these markers, except for CD86⁺, is observed qualitatively in L-GAS compared with S- and M-GAS. FIG. 81 top right and top left present the area fraction occupied by α-SMA⁺ and CD31⁺ cells, respectively. The area covered by α-SMA⁺ cells in L-GAS is ˜1.8 and 3.3 times higher than that in M-GAS and S-GAS, respectively. Furthermore, the area covered by CD31⁺ cells in L-GAS is ˜1.9 and 3.3 times higher than that in M-GAS and S-GAS, respectively. In both cases, the larger the microparticle, the higher the myofibroblast and endothelial cell infiltration. FIG. 81 middle left shows the CD68⁺ cell area in S-, M-, and L-GAS. Similar to the trends observed with CD31⁺ and α-SMA⁺ cells, the coverage area by CD68⁺ macrophages in L-GAS is ˜1.6 and 2.9 times higher than that in M- and S-GAS, respectively; however, there is no significant statistical difference between S-GAS and M-GAS. FIG. 81 middle right shows that CD86⁺ M1 macrophages coverage area is similar in the study groups. This result is consistent with the previously observed M1 macrophage infiltration in subcutaneously implanted GHS, fabricated using varying hydrogel microparticle sizes. FIG. 81 bottom left presents the area of CD206⁺ cells, showing that there are ˜1.4 and 1.3 times higher M2 macrophages in L-GAS than those in M- and S-GAS, respectively. While the area covered by CD206⁺ cells is larger in L-GAS, there is no significant difference between S- and M-GAS. FIG. 103 presents the comparison between the coverage area of CD86⁺ and CD206⁺ in GAS, fabricated using the same microparticle sizes. In S- and L-GAS, the CD206⁺ coverage area is higher than that of CD86⁺, and no significant difference is observed for the coverage area of these cells in M-GAS. This implies a shift from the pro-inflammatory to anti-inflammatory phenotypes after 1 week of GAS implantation, a transition already described in tissue regeneration applications for other biomaterials. It has been shown that inducing elongation to macrophages promotes a pro-regenerative M2 phenotype. In the field of granular scaffolds, pore size influences cell elongation and the M1-to-M2 transition. At the two extremes of confinement, scaffolds made of large microparticles (e.g., 130 μm) allow greater freedom for cell elongation, whereas those made of smaller microparticles (e.g., 40 μm) cause over-confinement, forcing cells to stretch and adapt the geometry of smaller pores. Scaffolds made of medium-sized microparticles (e.g., 70 μm), however, impose a spatial confinement that limits cell elongation, restricting the M1-to-M2 transition. Together, these findings support our observations, which show a more pronounced M1-to-M2 transition in S- and L-GAS, compared with M-GAS. FIG. 81 bottom right shows the coverage area of CD11b⁺ cells in S-, M-, and L-GAS, where L-GAS being ˜2.0 and 2.4 times greater than M- and S-GAS, respectively. The higher CD206⁺ and CD11b⁺ cell coverage area in L-GAS is likely a result of larger pores. Overall, these results show that increasing pore size by using larger microparticles in GAS within our experimental range leads to higher cell infiltration. Furthermore, myofibroblasts, macrophages, leukocytes, and endothelial cell recruitment in GAS is regulated by pore size, which may be further engineered for tissue regeneration.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the apparatus and methods of using and making the same disclosed herein have been discussed and illustrated, it is to be distinctly understood that the invention is not limited thereto but may be otherwise variously embodied and practiced within the scope of the following claims.

What is claimed is:

1. A method of forming a granular aerogel scaffold, the method comprising:
converting polymers or lipids to form hydrogel microparticles via a first crosslinking;
assembling the hydrogel microparticles to form a granular hydrogel scaffold via a second crosslinking; and
subjecting the granular hydrogel scaffold to supercritical carbon dioxide drying to form a granular aerogel scaffold.

2. The method of claim 1, wherein the method comprises converting polymers to form hydrogel microparticles via a first crosslinking.

3. The method of claim 2, wherein the polymers are selected from the group consisting of proteins, peptides, and carbohydrates.

4. The method of claim 2, wherein the polymers are gelatin methacryloyl.

5. The method of claim 1, wherein subjecting the granular hydrogel scaffold to supercritical carbon dioxide drying comprises:
replacing an aqueous phase of the granular hydrogel scaffold with an alcohol to form an alcogel; and
subjecting the alcogel to supercritical carbon dioxide drying to form the granular aerogel scaffold.

6. The method of claim 1, wherein the first crosslinking comprises physical crosslinking.

7. The method of claim 1, wherein the first crosslinking comprises chemical crosslinking.

8. The method of claim 1, wherein the second crosslinking comprises physical crosslinking.

9. The method of claim 1, wherein the second crosslinking comprises chemical crosslinking.

10. The method of claim 1, further comprising:
mixing the hydrogel microparticles with additional polymers and/or colloidal particles prior to assembling the hydrogel microparticles.

11. The method of claim 10, wherein the additional polymers are selected from the group consisting of aldehyde-modified carbohydrates, proteoglycans, and mixtures thereof.

12. The method of claim 1, further comprising:
decorating the hydrogel microparticles with one or more selected from the group consisting of biologics, colloidal particles, and hybrid biologics-colloids prior to assembling the hydrogel microparticles.

13. The method of claim 12, decorating the hydrogel microparticles comprises coating hydrogel microparticles with one or more selected from the group consisting of biologics, colloidal particles, and hybrid biologics-colloids.

14. The method of claim 12, wherein the biologics and/or colloidal particles and/or hybrid biologics-colloids are loaded to, attached on the surface of, or hybridized with nanocarriers bearing crosslinkable functional groups.

15. The method of claim 1, further comprising:
encapsulating one or more selected from the group consisting of biologics, colloidal particles, and hybrid biologics-colloids in the hydrogel microparticles.

16. A granular aerogel scaffold formed from the method of claim 1.
